(19) United States
(12) Patent Application Publication
(10) Pub. No.: US 2009/0203610 A1
(43) Pub. Date: Aug. 13, 2009

(54) METHODS OF TREATING OBESITY OR DIABETES USING NT-4/5

(75) Inventors: Arnon Rosenthal, Woodside, CA (US); John C. Lin, Palo Alto, CA (US); Jennifer R. Stratton, Belmont, CA (US); Robert D. Klein, Palo Alto, CA (US); Jaume Pons, San Bruno, CA (US)

Correspondence Address: PFIZER INC, 10555 SCIENCE CENTER DRIVE, SAN DIEGO, CA, 92121, US

(73) Assignee: RINAT NEUROSCIENCE CORPORATION

(21) Appl. No.: 12/404,202

(22) Filed: Mar. 13, 2009

Related U.S. Application Data
(63) Continuation of application No. 11/064,309, filed on Feb. 22, 2005, now abandoned.
(60) Provisional application No. 60/546,390, filed on Feb. 20, 2004.

(51) Int. Cl. A61K 38/12

(52) U.S. Cl. 514/12

(57) ABSTRACT

The invention concerns methods for treating obesity, non-insulin dependent diabetes mellitus, metabolic syndrome, and other related diseases by administering an NT-4/5 polypeptide. The invention also concerns compositions and kits comprising an NT-4/5 polypeptide.

### CROSS-REFERENCE TO RELATED APPLICATION

This application is a continuation of U.S. patent application Ser. No. 11/064,309, filed Feb. 22, 2005, which claims the priority benefit of the provisional patent application U.S. Ser. No. 60/546,390, filed Feb. 20, 2004, each of which is incorporated herein by reference in its entirety.

### FIELD OF THE INVENTION

The invention concerns use of NT-4/5 polypeptides in the treatment and/or prevention of obesity, non-insulin dependent (type II) diabetes mellitus, metabolic syndrome, or related diseases.

### BACKGROUND OF THE INVENTION

Obesity is a chronic disease and a major health concern in modern society. About 30% adults in U.S. are obese, and about 65% adults are overweight. Obesity is associated not only with a social stigma, but also with decreased life span and numerous health problems, including hypertension; type 2 diabetes mellitus; elevated plasma insulin concentrations; insulin resistance; dyslipidemia; hyperlipidemia; endometrial, breast, prostate and colon cancer; osteoarthritis; respiratory complications, such as obstructive sleep apnea; cholelithiasis; gallstones; arteriosclerosis; heart disease; abnormal heart rhythms; and heart arrythmias. Kopelman, P. G., Nature 404, 635-643 (2000).

Existing therapies for obesity include standard diets and exercise, very low calorie diets, behavioral therapy, pharmacotherapy involving appetite suppressants, thermogenic drugs, food absorption inhibitors, mechanical devices such as jaw wiring, waist cords and balloons, and surgery. Jung and Chong, Clinical Endocrinology, 35: 11-20 (1991); Bray, Am. J. Clin. Nutr., 55: 538S-544S (1992). Protein-sparing modified fasting has been reported to be effective in weight reduction in adolescents. Lee et al., Clin. Pediatr., 31: 234-236 (April 1992). However, existing therapies are not very effective for a lot of obese patients. For the most severe obese patients, surgical intervention may be required. Considering the high prevalence of obesity in our society and the serious consequences associated therewith as discussed above, any therapeutic drug potentially useful in reducing weight of obese persons could have a profound beneficial effect on their health. There is a need for a drug that reduces total body weight of obese subjects toward their ideal body weight without significant adverse side effects and that helps the obese subject maintain the reduced weight level.

Patients with diabetes mellitus have impaired disposal of glucose either due to insufficient insulin production of the pancreatic β cells, i.e. insulin dependent diabetes mellitus (type I, IDDM), or due to insulin resistance of the target organs, i.e. non-insulin dependent diabetes mellitus (type II, NIDDM). More than 90% of patients of diabetes mellitus are patients afflicted with type II diabetes. Type II diabetes mellitus is often accompanied by hyperlipidemia, and an unusual high level of cholesterol or triglyceride (i.e., more than 220 mg/dl of total cholesterol, or more than 150 mg/dl of triglyceride) is a risk factor for arteriosclerosis and myocardial infarction. Type II diabetes mellitus is also often accompanied by obesity. Type II diabetes mellitus, hyperlipidemia and obesity are deeply related to each other; however, in order to improve each condition, multiple medications are needed. Dietary restriction and physical exercise can only improve glucose tolerance at the early stage of the disease, but at the later stages multiple classes of anti-hyperglycemic medications are often needed to contain glucose level. However, none of the current anti-hyperglycemic medicines are optimal. Patients often suffer from serious side effects and many eventually require insulin.

Neurotrophins are a family of small, homodimeric proteins, which play a crucial role in the development and maintenance of the nervous system. Members of the neurotrophin family include nerve growth factor (NGF), brain-derived neurotrophic factor (BDNF), neurotrophin-3 (NT-3), neurotrophin-4/5 (NT-4/5), neurotrophin-6 (NT-6), and neurotrophin-7 (NT-7). Neurotrophins, similar to other polypeptide growth factors, affect their target cells through interactions with cell surface receptors. According to current knowledge, two kinds of transmembrane glycoproteins serve as receptors for neurotrophins. Neurotrophin-responsive neurons possess a common low molecular weight (65-80 kDa), low affinity receptor (LNGFR), also known as p75NTR or p75, which binds NGF, BDNF, NT-3 and NT-4/5 with a K_{D} of 2×10⁻⁹ M; and large molecular weight (130-150 kDa), high-affinity (K_{D} in the 10⁻¹¹ M range) receptors, which are members of the trk family of receptor tyrosine kinases. The identified members of the Trk receptor family are TrkA, TrkB, and TrkC.

Both BDNF and NT-4/5 bind to the TrkB and p75NTR receptors with similar affinity. However, NT-4/5 and BDNF mutant mice exhibit quite contrasting phenotypes. Whereas NT-4/5^{-/-} mice are viable and fertile with only a mild sensory deficit, BDNF^{-/-} mice die during early postnatal stages with severe neuronal deficits and behavioral symptoms. Fan et al., Nat. Neurosci. 3(4):350-7, 2000; Liu et al., Nature 375:238-241, 1995; Conover et al., Nature 375:235-238, 1995; Ernfors et al., Nature 368:147-150, 1994; Jones et al., Cell 76:989-999, 1994. Several publications report that NT-4/5 and BDNF have distinct biological activities in vivo and suggest that the distinct activities may result partly from differential activation of the TrkB receptor and its down-stream signaling pathways by NT-4/5 and BDNF. Fan et al., Nat. Neurosci. 3(4):350-7, 2000; Minichiello et al., Neuron. 21:335-45, 1998; Wirth et al., Development. 130(23):5827-38, 2003; Lopez et al., Program No. 38.6, 2003 Abstract, Society for Neuroscience.

It has been shown that BDNF has blood glucose and blood lipid controlling activity and anti-obesity activity in type II diabetic model animals, C57 db/db mice. U.S. Pat. No. 6,391,312; Itakura et al., Metabolism 49:129-33 (2000). It has also been shown that BDNF has anti-obesity activity and activity of ameliorating leptin resistance in mice fed with high fat diet. U.S. Pub. No. 2003/0036512. It has also been reported that BDNF or NT-4/5 could transiently reverse the eating behavior and obesity in heterozygous BDNF knock out mice in which BDNF gene expression was reduced. Kernie et al., EMBO J. 19(6):1290-300, 2000. It has been reported that a de novo missense mutation of Y722C substitution on human TrkB results in impaired receptor phosphorylation and signaling to MAP kinase; and this mutation seems to result in a unique human syndrome of hyperphagic obesity. Yeo et al., Nat. Neurosci. 7:1187-1189 (2004).

All patents, patent applications, and publications cited herein are hereby incorporated by reference in their entirety.

### BRIEF SUMMARY OF THE INVENTION

The present invention is based on the discovery that NT-4/5 is effective in treating non-insulin dependent diabetes mellitus (type II diabetes) in diabetic animal models, and an associated effect of the NT-4/5 treatment is reduction of body weight, food intake, and triglyceride levels. The present invention is also based on the discovery that NT-4/5 is effective in reducing body weight, controlling food intake, controlling blood glucose level, and reducing triglyceride in normal animals and obese animal models.

In one aspect, the invention provide methods for treating non-insulin dependent diabetes mellitus in an individual comprising administering an effective amount of an NT-4/5 polypeptide to the individual.

The invention also provides methods for treating any of one or more of hyperglycemia, low glucose tolerance, insulin resistance, abdominal obesity, lipid disorder, dyslipidemia, hyperlipidemia, hypertriglyceridemia, and metabolic syndrome in an individual comprising administering an effective amount of an NT-4/5 polypeptide to the individual. Treatment of any of these disorders may be in association with treating non-insulin dependent diabetes mellitus.

In another aspect, the invention provides methods for controlling blood glucose levels in an individual having non-insulin dependent diabetes mellitus or at risk of non-insulin dependent diabetes mellitus comprising administering an effective amount of an NT-4/5 polypeptide to the individual. In some embodiments, the individual is human and has an HbA1c value of about 7%, about 8%, about 9%, about 10%, or above. In some embodiments, the blood glucose level in the individual is reduced and generally maintained within the normal range.

In another aspect, the invention provides methods for controlling blood triglyceride levels in an individual having non-insulin dependent diabetes mellitus or at risk of non-insulin dependent diabetes mellitus comprising administering an effective amount of an NT-4/5 polypeptide to the individual. In some embodiments, the blood triglyceride level in the individual is reduced and generally maintained within the normal range.

In another aspect, the invention provides methods for improving insulin resistance in an individual having non-insulin dependent diabetes mellitus or is at risk of non-insulin dependent diabetes mellitus comprising administering an effective amount of an NT-4/5 polypeptide to the individual. In some embodiments, blood insulin level in the individual is reduced and generally maintained within the normal range.

In another aspect, the invention provides methods for controlling body weight in an individual having non-insulin dependent diabetes mellitus or at risk of non-insulin dependent diabetes mellitus comprising administering an effective amount of an NT-4/5 polypeptide to the individual. The invention also provides methods for maintaining body weight or preventing weight gain in an individual having non-insulin dependent diabetes mellitus or at risk of non-insulin dependent diabetes mellitus comprising administering an effective amount of an NT-4/5 polypeptide to the individual. In some embodiments, the body weight is controlled or maintained due to a loss of body fat content. In some embodiments, the body weight in the individual is reduced and generally maintained within the normal range.

In another aspect, the invention provides methods for controlling body fat content in an individual having non-insulin dependent diabetes mellitus or at risk of non-insulin dependent diabetes mellitus comprising administering an effective amount of an NT-4/5 polypeptide to the individual. In some embodiments, lean mass content of the individual is generally maintained during treatment.

In another aspect, the invention provides methods for controlling food intake in an individual having non-insulin dependent diabetes mellitus or at risk of non-insulin dependent diabetes mellitus comprising administering an effective amount of an NT-4/5 polypeptide to the individual.

In another aspect, the invention provides methods for delaying the development of non-insulin dependent diabetes mellitus in an individual comprising administering an effective amount of an NT-4/5 polypeptide to the individual. In some embodiments, the onset of the disease (for example, onset of diabetes from a pre-diabetes condition) is delayed. In other embodiments, the progression of the diabetes (for example, development of diabetic complications) is delayed. In some embodiments, the individual is at risk of non-insulin dependent diabetes mellitus. In some embodiments, development of non-insulin dependent diabetes mellitus in the individual is delayed or prevented. In some embodiments, the development of complications associated with non-insulin dependent diabetes mellitus in the individual is delayed or prevented.

The invention also provides methods for treating obesity (including managing obesity) in an individual comprising administering an effective amount of an NT-4/5 polypeptide to the individual. In some embodiments, the individual does not have genetic deficiency of BDNF. In some embodiments, the obesity is associated with leptin-resistance. In some embodiments, the obesity is associated with non-insulin dependent diabetes mellitus. In some embodiments, the obesity is associated with NT-4/5 resistance.

The invention also provides methods for treating any of one or more of hyperglycemia, low glucose tolerance, insulin resistance, lipid disorder, dyslipidemia, hyperlipidemia, hypertriglyceridemia, and metabolic syndrome in an individual comprising administering an effective amount of an NT-4/5 polypeptide to the individual. Treatment of any of these disorders may be in association with treating obesity.

The invention also provides methods for controlling body weight in an individual comprising administering an effective amount of an NT-4/5 polypeptide to the individual. The invention also provides methods for maintaining body weight or preventing weight gain in an individual comprising administering an effective amount of an NT-4/5 polypeptide to the individual. In some embodiments, the individual is obese. In some embodiments, the individual does not have genetic deficiency of BDNF. In some embodiments, the individual is at risk of obesity or is overweight.

The invention also provides methods for increasing metabolic rate in an individual comprising administering an effective amount of an NT-4/5 polypeptide to the individual. In some embodiments, the individual is obese. In some embodiments, the individual does not have genetic deficiency of BDNF. In some embodiments, the individual is at risk of obesity or is overweight.

The invention also provides methods for controlling food intake in an individual comprising administering an effective amount of an NT-4/5 polypeptide to the individual. In some embodiments, the individual is obese. In some embodiments, the individual does not have genetic deficiency of BDNF. In some embodiments, the individual is at risk of obesity or is overweight.

The invention also provides methods for controlling blood triglyceride levels in an individual comprising administering an effective amount of an NT-4/5 polypeptide to the individual. In some embodiments, the individual is obese. In some embodiments, the individual does not have genetic deficiency of BDNF. In some embodiments, the individual is at risk of obesity or is overweight.

The invention also provides methods for controlling blood glucose level in an individual comprising administering an effective amount of an NT-4/5 polypeptide to the individual. In some embodiments, the individual is obese. In some embodiments, the individual does not have genetic deficiency of BDNF. In some embodiments, the individual is at risk of obesity or is overweight.

The invention also provides methods for reducing body fat content in an individual in need of weight loss comprising administering an effective amount of an NT-4/5 polypeptide to the individual. The treatment may not result in significant change of lean mass content in the individual. In some embodiments, the individual is obese. In some embodiments, the individual does not have genetic deficiency of BDNF. In some embodiments, the individual is at risk of obesity or is overweight.

The invention also provides methods for delaying the development of obesity in an individual comprising administering an effective amount of an NT-4/5 polypeptide to the individual. In some embodiments, the onset of the obesity is delayed. In other embodiments, the progression of the obesity (for example, development of obesity associated complications, increase of BMI) is delayed. In some embodiments, development of obesity in the individual is prevented. In some embodiments, the development of complications associated with obesity in the individual is prevented. In some embodiments, the individual is at risk of obesity or is overweight.

The invention also provides methods for treating metabolic syndrome in an individual comprising administering an effective amount of an NT-4/5 polypeptide to the individual. The invention also provides methods of delaying the development of metabolic syndrome in an individual comprising administering an effective amount of an NT-4/5 polypeptide to the individual. In some embodiments, the metabolic syndrome is prevented. In some embodiments, the individual is at risk of developing metabolic syndrome.

The invention also provides methods for preventing bone loss in an individual comprising administering an effective amount of an NT-4/5 polypeptide to the individual. In some embodiments, the bone density in the individual is generally maintained. In some embodiments, the individual has osteoporosis.

The invention also provides methods for reducing thyroid hormone in an individual comprising administering an effective amount of an NT-4/5 polypeptide to the individual. In some embodiments, the individual has hyperthyroidism.

In any of the methods described herein, the NT-4/5 polypeptide may be administered in conjunction with an antibody that specifically binds to the NT-4/5 polypeptide. In some embodiments, administration in conjunction with the antibody enhances the therapeutic efficacy of the N-4/5 polypeptide. In some embodiments, administration in conjunction with the antibody reduces the effect amount of the NT-4/5 required for the treatment. In some embodiments, administration in conjunction with the antibody reduces the frequency of the NT-4/5 administration. The NT-4/5 polypeptide and the antibody may be administered simultaneously or sequentially, or may be administered in a co-formulation. In some embodiments, the binding of the antibody to the NT-4/5 polypeptide does not significantly interfere with binding of the NT-4/5 polypeptide to TrkB receptor.

Administration of NT-4/5 polypeptide described herein may be by any means known in the art, including: parenterally, intravenously, subcutaneously, via inhalation, intraarterially, intramuscularly, intracardially, intraventricularly, intrathecally, intraperitoneally, and transdermally.

Any NT-4/5 polypeptide described herein may be used for any of the methods described above. In some embodiments, the NT-4/5 polypeptide is a naturally occurring NT-4/5 (interchangeably termed “NT-4/5” herein). In some embodiments, the NT-4/5 polypeptide is a mature human NT-4/5 (SEQ ID NO:1). In some embodiments, the NT-4/5 polypeptide is a naturally occurring allelic variant of mature human NT-4/5 (SEQ ID NO:1). In some embodiments, the NT-4/5 polypeptide comprises a sequence of a naturally occurring NT-4/5 with one or more amino acid substitutions. In some embodiments, the NT-4/5 polypeptide is linked to one or more PEG molecules. In some embodiments, the NT-4/5 polypeptide is linked to one or more PEG molecules at the N-terminal of the NT-4/5 polypeptide. In some embodiments, the NT-4/5 polypeptide comprises the amino acid sequence of SEQ ID NO:1 with amino acid residue G at position 1 changed to S or T, and wherein the NT-4/5 polypeptide is linked to a PEG molecule at position 1. In some embodiments, the NT-4/5 polypeptide comprises the amino acid sequence of SEQ ID NO:1 with amino acid residue S at position 50 changed to C, and wherein the NT-4/5 polypeptide is linked to a PEG molecule at position 50.

In another aspect, the invention provides pharmaceutical compositions comprising an effective amount of an NT-4/5 polypeptide, wherein the NT-4/5 polypeptide is linked to one or more PEG molecules. In some embodiments, the NT-4/5 polypeptide comprises one or more amino acid substitutions as compared to a naturally occurring NT-4/5 sequence and the substituted amino acids are linked to the one or more PEG molecules. In some embodiments, the NT-4/5 polypeptide comprises the amino acid sequence of SEQ ID NO:1 with amino acid residue G at position 1 changed to S or T, and wherein the NT-4/5 polypeptide is linked to a PEG molecule at position 1. In some embodiments, the NT-4/5 polypeptide comprises the amino acid sequence of SEQ ID NO:1 with an amino acid residue substitution, and wherein the NT-4/5 polypeptide is linked to a PEG molecule at the substituted position (e.g., S at position 50 changed to C).

In another aspect, the invention provides compositions comprising an effective amount of an NT-4/5 polypeptide and an antibody that specifically binds to the NT-4/5 polypeptide. In some embodiments, the NT-4/5 polypeptide and the antibody are present in a predetermined ratio. In some embodiments, the binding of the antibody to the NT-4/5 polypeptide does not significantly interfere with binding of the NT-4/5 polypeptide to TrkB receptor.

In another aspect, the invention provides kits comprising an NT-4/5 polypeptide for use in any of the methods described herein. In some embodiments, the kit comprises a container, a composition comprising an NT-4/5 polypeptide described herein, in combination with a pharmaceutically acceptable carrier, and instructions for using the composition in any of the methods described herein. The kits may further comprise an antibody that specifically binds to the NT-4/5 polypeptide and an instruction for administering the antibody in conjunction with the NT-4/5 polypeptide.

Methods described herein are generally for treating an individual in need of the treatment.

### BRIEF DESCRIPTION OF THE SEVERAL VIEWS OF THE DRAWING(S)

FIG. 1 is a graph showing the effect of NT-4/5 on blood glucose levels in db/db mice. Blood glucose levels are expressed as mean±SEM (nM). “*” indicates a statistically significant difference (p<0.05) as compared to vehicle. The X axis corresponds to days since the experiment started and the Y axis corresponds to blood glucose concentration (mM).

FIG. 2 is a graph showing the effects of NT 4/5 on HbA1c level in db/db mice. HbA1c values are expressed as mean±SEM (%). “*” indicates a statistically significant difference (p<0.05) as compared to vehicle. The X axis corresponds to days since the experiment started and the Y axis corresponds to HbA1c (%).

FIG. 3 is a graph showing the effect of NT-4/5 on insulin resistance in db/db mice. Blood insulin levels are expressed as mean±SEM (nM). “*” indicates a statistically significant difference (p<0.05) as compared to vehicle. The X axis corresponds to days since the experiment started and the Y axis corresponds to insulin level (nM).

FIG. 4 is a graph showing the effect of NT-4/5 on obesity in db/db mice. Body weight is expressed as mean±SEM (g). “*” indicates a statistically significant difference (p<0.05) as compared to vehicle. The X axis corresponds to days since the experiment started and the Y axis corresponds to body weight (g).

FIG. 5 is a graph showing the effect of NT 4/5 on food intake in db/db mice. Food intake is expressed as mean±SEM (g/kg/day). “*” indicates a statistically significant difference (p<0.05) as compared to vehicle. The X axis corresponds to days since the experiment started and the Y axis corresponds to food intake (g/kg/day).

FIG. 6A are graphs showing the blood glucose levels of db/db mice treated with different doses of NT-4/5 at T0 (baseline, left) and T6 (right). The X axis corresponds to NT-4/5 doses (mg/kg) and the Y axis corresponds to blood glucose concentration (mg/dL).

FIG. 6B are graphs showing the blood triglyceride levels of db/db mice treated with different doses of NT-4/5 at T0 (baseline, left) and T6 (right). The X axis corresponds to NT-4/5 doses (mg/kg) and the Y axis corresponds to blood triglyceride concentration (mg/dL).

FIG. 6C are graphs showing the blood cholesterol levels of db/db mice treated with different doses of NT-4/5 at T0 (baseline, left) and T6 (right). The X axis corresponds to NT-4/5 doses (mg/kg) and the Y axis corresponds to blood cholesterol concentration (mg/dL).

FIG. 7 is a graph showing the dose response of NT-4/5 on the food intakes of db/db mice. “*” indicates a statistically significant difference (p<0.05) as compared to the vehicle. The X axis corresponds to days since the experiment started and the Y axis corresponds to food intake (g). The different doses of NT-4/5 are indicated in the graph.

FIG. 8A is a graph showing the effect of different dosages of NT-4/5 on body weights of db/db mice. “*” indicates a statistically significant difference (p<0.05) as compared to the vehicle. The X axis corresponds to days since the experiment started and the Y axis corresponds to body weight (g). The different doses of NT-4/5 are indicated in the graph.

FIG. 8B is a graph showing the effect of different dosages of NT-4/5 on the percentage of body weight changes in db/db mice. “*” indicates a statistically significant difference (p<0.01) as compared to the vehicle. The X axis corresponds to days since the experiment started and the Y axis corresponds to the percentage of body weight change in NT-4/5 treated mice. The different doses of NT-4/5 are indicated in the graph.

FIG. 9A is a graph showing the effect of different dosages of NT-4/5 on non-fasting blood glucose level in polygenic obese and diabetic mice (NONcNZO-10). Black bars on the X-axis indicate the treatment period. “*” indicates a statistically significant difference of P<0.05 as compared to the vehicle; “**” indicates a statistically significant difference of P<0.01 as compared to the vehicle; and “***” indicates a statistically significant difference of P<0.001 as compared to the vehicle. Two way ANOVA followed by Bonferroni post-tests were used for statistical analysis.

FIG. 9B is a graph showing the effect of different dosages of NT-4/5 on HbA1c level on day 30 in polygenic obese and diabetic mice (NONcNZO-10). One way ANOVA (F=7.249, P=0.0057), followed by Dunnett's post-tests were used for statistical analysis. P values are relative to the vehicle group as indicated in the graph.

FIGS. 10A and 10B are graphs showing the effect of different dosages of NT-4/5 on body weight (FIG. 10A) and food intake (FIG. 10B) in polygenic obese and diabetic mice (NONcNZO-10). Black bars on the X-axis indicate the treatment period.

FIGS. 11A and 11B are graphs showing the effect of different dosages of NT-4/5 on body weight (FIG. 11A) and food intake (FIG. 11B) in high fat diet-induced obese mice (DIO mice).

FIGS. 12A and 12 B are graphs showing the effect of different dosages of NT-4/5 on fasting glucose (FIG. 12A) and oral glucose tolerance (FIG. 12B) in high fat diet-induced obese mice (DIO mice) which were diabetic. Fasting glucose level and oral glucose tolerance tests were performed on day 60 after three daily doses of NT-4/5.

FIG. 13 is a graph showing the receptor tyrosine phosphorylation in a TrkB expressing cell line by different concentrations of PEG-NT4-S50C(S50C.2) or NT-4/5.

FIGS. 14A and 14B are graphs showing the effect of a single subcutaneous injection of 1 mg/kg PEG-NT4-S50C on body weight (FIG. 14A) and food intake (FIG. 14B) in polygenic obese and diabetic mice (NONcNZO-10). Vertical arrow indicates the time of the single injection of either NT-4/5 or PEG-NT4-S50C. Horizontal bars indicate the period of the daily subcutaneous injection for wild type NT-4/5. Two way ANOVA followed by Bonferroni's post-test were used for statistical analysis. “*” indicates a statistically significant difference of P<0.05 as compared to the vehicle; “**” indicates a statistically significant difference of P<0.01 as compared to the vehicle; and “***” indicates a statistically significant difference of P<0.001 as compared to the vehicle.

FIG. 15 is a graph showing the receptor tyrosine phosphorylation in a TrkB expressing cell line by different concentrations of 1PEG-NT4-G1S (G1S 1PEG), 2PEG-NT4-G1S (G1S 2PEG), or NT-4/5.

FIG. 16 is a graph showing the serum half life of 2PEG-NT4-G1S and NT-4/5 (NT4) after a single subcutaneous dose of 4 mg/kg in the db/db mice.

FIG. 17 is a graph showing the effect of a single subcutaneous injection of N-4/5, 2PEG-NT4-G1S, and 1PEG-NT4-G1S on non-fasting serum glucose levels of db/db mice.

FIG. 18 is a graph showing the changes of body weight after weekly dosing of wild type (WT) NT-4/5 (referred to as WT NT4) versus pegylated G1S NT-4/5 (referred to as Peg G1S NT4) in DIO mice. The data are expressed in mean values with the error bars representing the standard errors of means (SEM). The treatment groups differed significantly by 2 way ANOVA (F=207.01, P<0.0001).

FIG. 19 is a graph showing the changes of food intake after weekly dosing of wildtype NT-4/5 (referred to as WT NT4) versus pegylated G1S NT-4/5 (referred to as PEG NT4) in DIO mice. The data are expressed in mean values with the error bars representing the standard errors of means (SEM). “*” indicates P<0.05 and “**” indicates P<0.01 for the corresponding time point and treatment type as compared to vehicle control group.

FIG. 20 is a graph showing effect of daily subcutaneous injection of NT-4/5 (10 mg/kg) to the respiratory quotient (RQ=Vco₂/Vo₂, i.e. ratio of carbon dioxide production over oxygen consumption measured by CCMS) in DIO mice. The individual data points are expressed (P=0.0025, Student t-test).

FIG. 21 is a graph showing the effect of daily subcutaneous injection of NT-4/5 (10 mg/kg) on the body weight (panel A), food intake (panel B), body fat content (panel C), and percent body lean mass (panel D) in DIO mice. Data are expressed in means with error bars representing SEM. Statistical analysis was done by 2-way ANOVA with pairwise comparisons by Bonferroni posttests (* P<0.05; ** P<0.01; *** P<0.001).

FIG. 22 is a graph showing the effect of weekly intravenous injection of NT-4/5 (2 mg/kg) and BDNF (2 mg/kg) on the body weight in DIO mice. Data are expressed in means with error bars representing SEM. Statistical analysis was done by 2-way ANOVA with pairwise comparisons by Bonferroni posttests. “*” indicates P<0.05; “**” indicates P<0.01; and “***” indicates P<0.001 for the corresponding time point and treatment type as compared to vehicle control group.

FIG. 23 is a graph showing the effect of weekly intravenous injection of NT-4/5 (2 mg/kg) and BDNF (2 mg/kg) on the food intake in DIO mice. Data are expressed in means with error bars representing SEM. Statistical analysis was done by 2-way ANOVA with pairwise comparisons by Bonferroni posttests. “*” indicates P<0.05; “**” indicates P<0.01; and “***” indicates P<0.0011 for the corresponding time point and treatment type as compared to vehicle control group.

FIG. 24 is a graph showing the effect of daily subcutaneous injection of NT-4/5 (1, 2, 5 and 10 mg/kg) on the body weight in lean male C57BL/6 mice. Data are expressed in means with error bars representing SEM. Statistical analysis was done by 2-way ANOVA with pairwise comparisons by Bonferroni posttests of each treatment relative to the vehicle control. “*” indicates P<0.05; “**” indicates P<0.01; and “***” indicates P<0.001 for the corresponding time point and treatment type as compared to vehicle control group.

FIG. 25 is a graph showing the effect of daily subcutaneous injection of NT-4/5 (1, 2, 5 and 10 mg/kg) on the food intake in lean male C57BL/6 mice. Data are expressed in means with error bars representing SEM. Statistical analysis was done by 2-way ANOVA with pairwise comparisons by Bonferroni posttests of each treatment relative to the vehicle control “*” indicates P<0.05; “**” indicates P<0.01; and “***” indicates P<0.001 for the corresponding time point and treatment type as compared to vehicle control group.

FIG. 26 is a graph showing the effect of daily subcutaneous injection of NT-4/5 (1, 2, 5 and 10 mg/kg) on nonfasting blood glucose in lean male C57BL/6 mice. Data are expressed in means with error bars representing SEM. Statistical analysis was done by 2-way ANOVA with pairwise comparisons by Bonferroni posttests of each treatment relative to the vehicle control. “*” indicates P<0.05; “**” indicates P<0.01; and “***” indicates P<0.001 for the corresponding time point and treatment type as compared to vehicle control group.

FIG. 27 is a graph showing percentage survival of db/db female mice after daily subcutaneous injection of NT-4/5 (20 mg/kg/day) from day 1 to day 26 as compared to the vehicle control group.

FIG. 28 are graphs showing effect of NT-4/5 on long term (daily subcateneous injections from day 1 to day 26) glucose control in db/db mice. Panel A shows the level of HbA1c on day 45 (15 days post NT-4/5 dosing). Panel B shows the glucose tolerance test performed on day 54 (28 days post NT-4/5 dosing). Panel C shows serum glucose level from day 0 to day 30. Panel D shows blood insulin level from day 0 to day 20.

FIG. 29A is a graph showing the effect of NT4/5 and/or Mab 1241 on body weights in db/db mice. Body weights are expressed as mean±SEM (% of baseline). “*” indicates a statistically significant difference (p<0.05) as compared to vehicle, “**” represents p<0.01; “***” represents p<0.001. The X axis corresponds to days since the experiment started and the Y axis corresponds to body weight (% of baseline).

FIG. 29B is a graph showing the effect of NT4/5 and/or Mab 1241 on food intake in db/db mice. Food intake is expressed as mean±SEM (g). “*” indicates a statistically significant difference (p<0.05) as compared to vehicle, “**” represents p<0.01; “***” represents p<0.001. The X axis corresponds to days since the experiment started and the Y axis corresponds to food intake (g).

FIG. 29C is a graph showing the effect of NT4/5 and/or Mab 1241 on blood glucose levels in db/db mice. Blood glucose levels are expressed as mean±SEM (mg/dL). “*” indicates a statistically significant difference (p<0.05) as compared to vehicle, “**” represents p<0.01; “***” represents p<0.001. The X axis corresponds to days since the experiment started and the Y axis corresponds to food intake (g). The X axis corresponds to days since the experiment started and Y axis corresponds to blood glucose concentration (mg/dL).

### DETAILED DESCRIPTION OF THE INVENTION

The present invention is based on the discovery that administration of a therapeutically effective amount of an NT-4/5 polypeptide may be used for treating obesity and non-insulin dependent diabetes mellitus.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### DEFINITIONS

As used herein, “treatment” is an approach for obtaining beneficial or desired clinical results. For purposes of this invention, beneficial or desired clinical results include, but are not limited to, one or more of the following: improving, lessening severity, alleviation of one or more symptoms associated with a disease. For example, for non-insulin dependent diabetes mellitus, beneficial or desired clinical results include any of improvement of blood glucose control, blood lipid control, and/or insulin resistance. In some embodiments, beneficial or desired clinical results may include improving, lessening severity, and/or alleviation any of the disorders associated with non-insulin dependent diabetes mellitus, such as hyperglycemia, low glucose tolerance, insulin resistance, hyperinsulinemia, obesity, lipid disorders, dyslipidemia, hyperlipidemia, hypertriglyceridemia, hypercholesterolemia, metabolic syndrome, diabetic nephropathy, diabetic neuropathy, diabetic retinopathy, diabetic microvasculopathy and other associated diseases known and described herein. For obesity, beneficial or desired clinical results include any of reducing or maintaining body weight; controlling (including reducing) food intake or calorie intake; increasing metabolic rate or inhibiting reduction of metabolic rate; and improving, lessening severity, and/or alleviation any of the disorders associated with obesity, such as diabetes, non-insulin dependent diabetes mellitus, hyperglycemia, low glucose tolerance, insulin resistance, lipid disorder, dyslipidemia, hyperlipidemia, hypertriglyceridemia, hypercholesterolemia, abdominal obesity, eating disorder, metabolic syndrome, hypertension, osteoarthritis, myocardia infarction, stroke and other associated diseases known and described herein; increasing the quality of life of those suffering from the obesity, and/or prolonging lifespan.

An “effective amount” is an amount sufficient to effect beneficial or desired clinical results including improving, alleviation and/or reduction one or more symptoms associated with a disease. For treating non-insulin dependent diabetes mellitus, an effective amount of NT-4/5 is an amount sufficient to treat or ameliorate one or more symptoms associated with non-insulin dependent diabetes mellitus. An “effective amount” is an amount sufficient to result in one or more of the following (which can also correspond to various embodiments of the invention): reduce blood glucose levels and/or blood lipid levels; reduce hyperinsulinemia; improve insulin resistance; improve obesity and/or control of food intake accompanied diabetes. For treating obesity, an effective amount of an NT-4/5 polypeptide is an amount sufficient to treat or ameliorate one or more symptoms associated with obesity. An “effective amount” is an amount sufficient to result in one or more of the following (which can also correspond to various embodiments of the invention): reduce body weigh, control food intake, increase metabolic rate, decrease one or more symptoms resulting from the diseases associated with obesity, increase the quality of life of those suffering from the obesity, and/or prolong lifespan. An effective amount may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved. An “effective amount” may be given in one or more administrations.

As used herein, “delaying” development of type II diabetes means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease.

“Development” of type II diabetes means the onset and/or progression of the disease within an individual (which can be different embodiments of the invention). Type II diabetes development can be detectable using standard clinical techniques as described herein. However, development also refers to disease progression that may be initially undetectable. For purposes of this invention, progression refers to the biological course of the disease state, in this case, as determined by testing blood glucose, lipids, insulin levels, glucose tolerance test, Hb-A 1c level, as well as the onset and/or worsening of diabetic complications such as cardiovascular diseases, nephropathy, retinopathy and/or neuropathy. A variety of these diagnostic tests include, but are not limited to, blood pressure, resting and exercise electrocardiogram (EKG), echocardiogram, angiocardiogram, blood urea nitrogen (BUN), creatinine level, urinalysis, renal biopsy, glomerular filtration rate (GFR), opthalmoscopic examination, sensory and motor nerve conduction velocity, and peripheral nerve biopsy. “Development” includes occurrence, recurrence, and onset. As used herein “onset” or “occurrence” of type II diabetes includes initial onset and/or recurrence.

As used herein, an individual “at risk” of development of type II diabetes may or may not have detectable disease, and may or may not have displayed detectable disease prior to the treatment methods described herein. “At risk” denotes that an individual has one or more so-called risk factors, which are measurable parameters that correlate with development of type II diabetes. An individual having one or more of these risk factors has a higher probability of developing type II diabetes than an individual without these risk factor(s). These risk factors include, but are not limited to, age, diet, physical inactivity, metabolic syndrome, obesity, family history of obesity and/or diabetes, ethnicity (e.g. Pima Indians, Afro-American and Hispanics are at high risk), hereditary syndromes (e.g. maturity-onset diabetes of the young, MODY), history of previous disease (e.g. a history of gestational diabetes for women), presence of precursor disease (i.e. pre-diabetes).

The term “metabolic syndrome”, also known as syndrome X, is defined in the Third Report of the National Cholesterol Education Program Expert Panel on Detection, Evaluation and Treatment of High Blood Cholesterol in Adults (ATP-III). E. S. Ford et al., JAMA, vol. 287 (3), Jan. 16, 2002, pp 356359. Briefly, a person is defined as having metabolic syndrome if the person has three or more of the following disorders: abdominal obesity, hypertriglyceridernia, low HDL cholesterol, high blood pressure, and high fasting plasma glucose. The criteria for these are defined in ATP-III as: abdominal obesity as measured by waist circumference greater than 40 inches for men or greater than 35 inches for women; fasting blood triglycerides greater than or equal to 150 mg/dL; blood HDL cholesterol less than 40 mg/dL for men or less than 50 mg/dL for women; blood pressure greater than or equal to 130/85 mmHg; fasting glucose greater than or equal to 110 mg/dL.

Treatment of metabolic syndrome refers to the administration of an NT-4/5 polypeptide described herein to a subject with metabolic syndrome for obtaining beneficial or desired clinical results. For purposes of this invention, beneficial or desired clinical results include, but are not limited to, one or more of the following: improving, lessening severity, alleviation of one or more symptoms associated with the disease. For example, for treating metabolic syndrome, beneficial or desired clinical results include reducing any one or more of abdominal obesity, triglycerides, HDL cholesterol, blood pressure, and fasting glucose level.

As used herein, “delaying” development of metabolic syndrome means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease.

“Development” of metabolic syndrome means the onset and/or progression of the disease within an individual (which can be different embodiments of the invention). Metabolic syndrome development can be detectable using standard clinical techniques know in the art and as described herein. However, development also refers to disease progression that may be initially undetectable. For purposes of this invention, progression refers to the biological course of the disease state, in this case, as determined by measuring abdominal circumference, testing triglycerides, HDL, blood pressure, blood glucose, glucose tolerance test, Hb-A1c level, as well as the onset and/or worsening of metabolic syndrome complications such as cardiovascular diseases and diabetes. “Development” includes occurrence, recurrence, and onset. As used herein “onset” or “occurrence” of metabolic syndrome includes initial onset and/or recurrence.

As used herein, an individual “at risk” of development of metabolic syndrome may or may not have detectable disease, and may or may not have displayed detectable disease prior to the treatment methods described herein. For example, the individual may have two or less disorders or no disorders that define metabolic syndrome. “At risk” denotes that an individual has one or more so-called risk factors, which are measurable parameters that correlate with development of metabolic syndrome. An individual having one or more of these risk factors has a higher probability of metabolic syndrome than an individual without these risk factor(s).

As used herein, “controlling blood glucose level” or “improvement in blood glucose level” refers to reducing the blood glucose level in an individual (as compared to the level before treatment). In some embodiments, blood glucose level is generally maintained within the normal range. In some embodiments, the blood glucose level is reduced by at least about any of 10%, 20%, 30%, 40%, 50%, 60%, or 70% in the individual as compared to the level before treatment.

As used herein, “controlling blood lipid level” or “improvement in blood lipid level” refers to reducing the blood lipid level (such as blood triglyceride level, cholesterol level, and/or nonesterified fatty acid level) in an individual (as compared to the level before treatment). In some embodiments, the blood lipid level (such as blood triglyceride level, cholesterol level, and/or nonesterified fatty acid level) is generally maintained within the normal range. In some embodiments, the blood triglyceride level is reduced by at least about any of 10%, 20%, 30%, 40%, 50%, 60%, or 70% in the individual as compared to the level before treatment.

As used herein, “insulin resistance” refers to a condition wherein the insulin level to be required to exhibit insulin activity at the same level as a healthy person is much higher than that of the healthy person. That is, it means a condition wherein the activity of insulin or sensitivity for insulin is reduced. The target organ for insulin activity includes a liver, a muscle (skeletal muscle), and an adipose tissue. Insulin exhibits a gluconeogenesis suppression activity, a glucose release suppression activity, etc. in the liver. Insulin exhibits glucose-uptake promoting activity in the muscle (skeletal muscle) and adipose tissues. The translocation of glucose transportation carrier called GLUT4 from the cytoplasma to the surface of the cell membrane participates in the glucose-uptake. These activities may be measured for evaluating insulin resistance. The clinical evaluation of insulin resistance includes, for example, glucose tolerance test, euglycemic hyperinsulinemic clamp method (or “glucose clamp” in short), Homeostasis Model Assessment (HOMA, ref. Diabetes Care 23:57-63, 2000), etc.

As used herein, “improving insulin resistance” refers to reversing (which may be partial to complete) the reduced sensitivity of cells to insulin (for example, reduced sensitivity is manifested as any one or more of reduction of glucose-uptake into the peripheral tissues, enhancement of glycogenolysis, enhancement of gluconeogenesis, etc., all of which are observed in type II diabetes mellitus). For such improvement, the blood insulin level is reduced (and blood glucose level may be decreased). Improved insulin resistance may be assessed by glucose tolerance test, glucose clamp, or HOMA.

As used herein, “controlling body weight” or “improvement in body weight” refers to reducing the body weight in an individual (as compared to the level before treatment). In some embodiments, the body weight is generally maintained within the normal range. The body weight may be reduced by reducing the calorie intake and/or reducing the body fat accumulation. In some embodiments, the body weight is reduced at least about any of 5%, 10%, 20%, 30%, 40%, or 50% in the individual as compared to the level before treatment.

As used herein, “control food intake” refers to reducing the food intake in an individual (as compared to the level before treatment). In some embodiments, the food intake is generally maintained in the normal range. In some embodiments, the food intake is reduced by about any of 5%, 10%, 20%, 30%, 40%, 50%, or 60% in the individual as compared to the level before treatment.

As used herein, “obesity” is a condition in which there is an excess of body fat in a subject. Obesity may be due to any cause, whether genetic or environmental. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30.0 kg/m², or a condition whereby a subject with at least one co-morbidity has a BMI greater than or equal to 27.0 kg/m². An “obese subject” is an otherwise healthy subject with a Body Mass Index (BMI) greater than or equal to 30.0 kg/m² or a subject with at least one co-morbidity with a BMI greater than or equal to 27.0 kg/m². An obese subject may have a BMI of at least about any of 31.0, 32.0, 33.0, 34.0, 35.0, 36.0, 37.0, 38.0, 39.0, and 40.0. A “overweight subject” is a subject with a BMI of 25.0 to 29.9 kg/m².

Different countries may define obesity and overweight with different BMI. The term “obesity” is meant to encompass definitions in all countries. For example, the increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25.0 kg/m². In Asian countries, including Japan, an “obese subject” refers to a subject with at least one obesity-induced or obesity-related co-morbidity that requires weight reduction or that would be improved by weight reduction, with a BMI greater than or equal to 25.0 kg/m².

Obesity-induced or obesity-related co-morbidities include, but are not limited to, diabetes, non-insulin dependent diabetes mellitus-type II, impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

An individual “at risk” of obesity may or may not have detectable disease, and may or may not have displayed detectable disease prior to the treatment methods described herein. “At risk” denotes that an individual has one or more so-called risk factors, which are measurable parameters that correlate with development of obesity. An individual having one or more of these risk factors has a higher probability of being obese than an individual without these risk factor(s). These risk factors include, but are not limited to, age, diet, physical inactivity, metabolic syndrome, family history of obesity, ethnicity, hereditary syndromes, history of previous disease (e.g. eating disorder, metabolic syndrome, and obesity), presence of precursor disease (e.g., overweight). For example, an otherwise healthy individual with a BMI of 25.0 to less than 30.0 kg/m² or an individual with at least one co-morbidity with a BMI of 25.0 kg/m² to less than 27.0 kg/m² is at risk of obesity.

As used herein, “delaying” development of obesity means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. For example, one outcome of delaying development may be reducing the body weight of a subject at risk of obesity relative to that subject's body weight immediately before the administration of the polypeptides or the compositions described herein. Another outcome of delaying development may be preventing regain of body weight previously lost as a result of diet, exercise, or pharmacotherapy. Another outcome of delaying development may be preventing obesity from occurring if the treatment is administered prior to the onset of obesity in a subject at risk of obesity. Another outcome of delaying development may be decreasing the occurrence and/or severity of obesity-related disorders if the treatment is administered prior to the onset of obesity in a subject at risk of obesity.

“Development” of obesity means the onset and/or progression of the disease within an individual (which can be different embodiments of the invention). Obesity development can be detectable using standard clinical techniques as described herein. However, development also refers to disease progression that may be initially undetectable. For purposes of this invention, progression refers to the biological course of the disease state, in this case, as determined by assessing height and weight for estimating BMI, measuring waist circumference, assessing co-morbidities, as well as the onset and/or worsening of obesity complications such as arteriosclerosis, Type II diabetes, polycystic ovary disease, cardiovascular diseases, osteoarthritis, dermatological disorders, hypertension, insulin resistance, hypercholesterolemia, hypertriglyceridemia, and cholelithiasis. A variety of these diagnostic tests are known in the art. “Development” includes occurrence, recurrence, and onset. As used herein “onset” or “occurrence” of obesity includes initial onset and/or recurrence.

An “individual” or a “subject” is a mammal, more preferably a human. Mammals include, but are not limited to, farm animals, sport animals, pets, primates, horses, dogs, cats, mice and rats.

An individual having genetic deficiency of BDNF refers to an individual having a mutation at least one BDNF locus on the genome of the individual which results in lower level of expression of BDNF (e.g., BDNF transcripts in the hypothalamus) or biological activity as compared to an average level in the population of the same species who do not have the mutation. Lower level of expression or biological activity of BDNF may be assessed by other phenotype associated with lower level of expression or biological activity of BDNF, such as mental retardation, memory impairment, and/or learning deficiency. In some embodiments, the level of expression of BDNF or activity of BDNF in the individual having the mutation may be less than about any of 30%, 40%, 45%, 50%, 55%, 60%, 70% of the level or activity of the average of the same species. In some embodiments, the expression level of BDNF or biological activity is less than about 50%. In some embodiments, the individual has only one functional BDNF allele. In some embodiments, the individual is a mouse. In some embodiments, the individual is non-human. In some embodiments, the individual has obesity which is caused by the mutation at the BDNF locus on the genome of the individual.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), mutants thereof, fusion proteins comprising an antibody portion, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy chain constant domains that correspond to the different classes of immunoglobulins are called alpha, delta, epsilon, gamma, and mu, respectively. The subunit structures and three-dimensional configurations of different classes of immunoglobulins are well known.

It should be noted that, as used herein, the singular form “a”, “an”, and “the” includes plural references unless indicated otherwise.

### Methods of the Invention

In some embodiments, the invention provides methods for treating non-insulin dependent diabetes mellitus in an individual comprising administering an effective amount of an NT-4/5 polypeptide to the individual.

Non-insulin dependent diabetes mellitus (also termed Type 2 diabetes) is related to insulin resistance. Dignostic methods for non-insulin dependent diabetes mellitus is well known in the art.

In some embodiments, the invention provides methods for treating any of one or more of hyperglycemia, low glucose tolerance, insulin resistance, abdominal obesity, lipid disorder, dyslipidemia, hyperlipidemia, hypertriglyceridemia, and metabolic syndrome in an individual comprising administering an effective amount of an NT-4/5 polypeptide to the individual. Treatment of any of these disorders may be in association with treating non-insulin dependent diabetes mellitus.

In some embodiments, the invention provides methods for controlling blood glucose levels in an individual having non-insulin dependent diabetes mellitus or at risk of non-insulin dependent diabetes mellitus comprising administering an effective amount of an NT-4/5 polypeptide to the individual.

In some embodiments, the invention provides methods for controlling blood triglyceride levels in an individual having non-insulin dependent diabetes mellitus or at risk of non-insulin dependent diabetes mellitus comprising administering an effective amount of an NT-4/5 polypeptide to the individual.

In some embodiments, the invention provides methods for improving insulin resistance in an individual having non-insulin dependent diabetes mellitus or at risk of non-insulin dependent diabetes mellitus comprising administering an effective amount of an NT-4/5 polypeptide to the individual.

In some embodiments, the invention provides methods for controlling body weight in an individual having non-insulin dependent diabetes mellitus or at risk of non-insulin dependent diabetes mellitus comprising administering an effective amount of an NT-4/5 polypeptide to the individual.

In some embodiments, the invention provides methods for controlling food intake in an individual having non-insulin dependent diabetes mellitus or at risk of non-insulin dependent diabetes mellitus comprising administering an effective amount of an NT-4/5 polypeptide to the individual.

In some embodiments, the invention provides methods for delaying the development of non-insulin dependent diabetes mellitus in an individual comprising administering an effective amount of an NT-4/5 polypeptide to the individual. In some embodiments, the individual is at risk of non-insulin dependent diabetes mellitus. Individual having normal blood glucose level but abnormally high blood insulin level, or having blood glucose levels in the high normal range or glucose levels trending toward high may be treated with the NT-4/5 polypeptide to delay onset of diabetes. Diabetic complications may also be delayed by administration of the NT-4/5 polypeptide. In some embodiments, development of non-insulin dependent diabetes mellitus in the individual is prevented. In some embodiments, the development of complications associated with non-insulin dependent diabetes mellitus in the individual is prevented.

In some embodiments, the invention also provides methods for treating obesity in an individual comprising administering an effective amount of an NT-4/5 polypeptide to the individual. In some embodiments, the individual is human. In some embodiments, the individual does not have genetic deficiency of BDNF. Accordingly, the obesity may be due to any cause, whether genetic (except caused by genetic deficiency of BDNF) or environmental. In some embodiments, the obesity is associated with leptin-resistance. In some embodiments, the obesity is associated with non-insulin dependent diabetes mellitus. In some embodiments, the obesity is associated with NT-4/5 resistance. Treatment of obesity includes treating an individual whose body weight has been reduced after treatment and is no longer obese.

In some embodiments, the invention also provides methods for treating any of one or more of hyperglycemia, low glucose tolerance, insulin resistance, abdominal obesity, lipid disorder, dyslipidemia, hyperlipidemia, hypertriglyceridemia, and metabolic syndrome in an individual comprising administering an effective amount of an NT-4/5 polypeptide to the individual. Treatment of any of these disorders may be in association with treating obesity.

In some embodiments, the invention also provides methods for controlling body weight, controlling food intake, increasing metabolic rate, controlling blood glucose level, controlling triglyceride level, or reducing body fat content in an individual comprising administering an effective amount of an NT-4/5 polypeptide to the individual. In some embodiments, the individual is obese. In some embodiments the individual does not have genetic deficiency of BDNF. In some embodiments, the individual is at risk of obesity or is overweight.

In some embodiments, the invention also provides methods for delaying the development of obesity in an individual comprising administering an effective amount of an NT-4/5 polypeptide to the individual. In some embodiments, the onset of the obesity is delayed. In other embodiments, the progression of the obesity (for example, development of obesity associated complications, increase of BMI) is delayed. In some embodiments, the individual is at risk of obesity or is overweight. In some embodiments, development of obesity in the individual is prevented. In some embodiments, the development of complications associated with obesity in the individual is prevented.

In some embodiments, the invention also provides methods for treating metabolic syndrome in an individual comprising administering an effective amount of an NT-4/5 polypeptide to the individual. The invention also provides methods of delaying the development of metabolic syndrome in an individual comprising administering an effective amount of an NT-4/5 polypeptide to the individual. In some embodiments, the metabolic syndrome is prevented. In some embodiments, the individual is at risk of developing metabolic syndrome.

The invention also provides methods for preventing bone loss in an individual comprising administering an effective amount of an NT-4/5 polypeptide to the individual. In some embodiments, the bone density in the individual is generally maintained. In some embodiments, the individual has osteoporosis.

The invention also provides methods for reducing thyroid hormone in an individual comprising administering an effective amount of an NT-4/5 polypeptide to the individual. In some embodiments, the individual has hyperthyroidism.

In any of the methods described herein, the NT-4/5 polypeptide may be administered in conjunction with an antibody that specifically binds to the NT-4/5 polypeptide. In some embodiments, binding of the antibody to the NT-4/5 polypeptide does not generally interfere with binding of the NT-4/5 polypeptide to a TrkB receptor. For example, the binding of the NT-4/5 polypeptide to the TrkB receptor in the presence of the antibody may be reduced by less than about any of 5%, 10%, 20%, 30%, 40%, and 50% of the binding in the absence of the antibody. In some embodiments, the antibody competes with a TrkB receptor for binding to the NT-4/5 polypeptide, but the binding of the NT-4/5 to the TrkB receptor is not generally interfered by the antibody.

As used herein, administration in conjunction also encompasses administration as a co-formulation (i.e., the NT-4/5 polypeptide and the antibody are present (combined) in the same composition) and/or administration as separate compositions. “Administration in conjunction” is meant to encompass any circumstance wherein an NT-4/5 polypeptide and an antibody that specifically binds the NT-4/5 polypeptide are administered in an effective amount to an individual. It is understood that the NT-4/5 and the antibody can be administered at different dosing frequencies and/or intervals. For example, an NT-4/5 polypeptide can be administered daily to weekly, while the antibody can be administered less frequently. It is understood that the NT-4/5 and the antibody can be administered using the same route of administration or different routes of administration, and that different dosing regimens may change over the course of administration(s). Administration may be before the onset of obesity, non-insulin dependent diabetes mellitus, and/or other associated diseases.

The term “simultaneous administration,” as used herein, means that the antibody and the NT-4/5 polypeptide are administered with a time separation of no more than about 15 minutes, such as no more than about 10 minutes. When administered simultaneously, the antibody and the NT-4/5 polypeptide may be contained in the same dosage (e.g., a unit dosage form comprising both the neurotrophin antibody and the neurotrophin) or in discrete dosages (e.g., the antibody is contained in one dosage form and the NT-4/5 polypeptide is contained in another dosage form).

In one embodiment, the antibody and the NT-4/5 polypeptide are present in a single molecule. For example, a chimeric fusion protein may be made that comprises an antibody portion and an NT-4/5 polypeptide portion in a way that the antibody portion may bind and stabilize the NT-4/5 polypeptide portion of the chimeric protein.

In some embodiments, the antibody and the NT-4/5 polypeptide are administered in a predetermined ratio. Thus, in some embodiments, the ratio by weight of the antibody to the NT-4/5 polypeptide will be approximately 1 to 1. In some embodiments, this ratio may be between about 0.001 to about 1 and about 1 to about 1000, between about 0.01 to about 1 and about 1 to about 100, or between about 0.1 to about 1 and about 1 to about 10. Other ratios are contemplated.

Efficacy of NT-4/5 polypeptide treatment may be enhanced when at least one aspect of NT-4/5 polypeptide treatment is improved (as compared to NT-4/5 polypeptide treatment without co-administering the antibody). The improvement can be reflected in reduced frequency of NT-4/5 polypeptide treatment, reduced dosage of NT-4/5 polypeptide, or better relief of one or more symptoms associated with the disease to be treated. For example, treatment or prevention of diabetes by NT-4/5 polypeptide is enhanced by co-administration of an anti-NT4/5 polypeptide antibody when the co-administration of the antibody permits better relief (for example, when one or more symptoms of diabetes are better relieved by co-administration of antibody with NT-4/5 polypeptide than by administration of NT-4/5 polypeptide alone). The antibody may also reduce one or more side effects caused by NT-4/5 polypeptide administration. In some embodiments, enough antibody is administered so as to allow reduction of the normal dose of the NT-4/5 polypeptide by at least about 5%, at least about 10%, at least about 20%, or more. The reduction may be reflected in terms of amount administered at a given administration and/or amount administered over a given period of time (reduced frequency).

In any of the embodiments described above, the NT-4/5 polypeptide may be a naturally occurring mature NT-4/5, such as a human mature NT-4/5, any polypeptide fragment of naturally occurring mature NT-4/5, or modified forms thereof described herein.

Various formulations of NT-4/5 polypeptides may be used for administration. In some embodiments, the NT-4/5 polypeptide may be administered neat. In other embodiments, the NT-4/5 polypeptide and a pharmaceutically acceptable excipient are administered, and may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

Generally, these agents are formulated for administration by injection (e.g., intraperitoneally, intravenously, subcutaneously, intramuscularly, etc.), although other forms of administration (e.g., oral, mucosal, transdermal, inhalation, etc) can be also used. Administration can be systemic, e.g., intravenous and intraperitoneal, or localized. Accordingly, NT-4/5 polypeptide is preferably combined with pharmaceutically acceptable vehicles such as saline, Ringer's solution, dextrose solution, and the like.

Different methods may be used to facilitate the delivery of NT-4/5 polypeptide to the brain. These methods include: (a) neurosurgical-based (intraventricular drug infusion, hyperosmotic opening of the blood brain barrier (BBB)); (b) pharmacological-based (peptide lipidization, liposomes); and (c) physiological-based (biochemical opening of the BBB, chimeric peptides). Chimeric peptides can be formed by the covalent coupling of NT-4/5 polypeptide to a brain transport vector that undergoes absorptive-mediated or receptor-mediated transcytosis through the BBB. For example, a brain transport vector can be a monoclonal antibody to the transferrin receptor. See Pardridge et al. Pharmacol. Toxicol. 71:3-10 (1992).

The particular dosage regimen, i.e., dose, timing and repetition, will depend on the particular individual and that individual's medical history. Generally, any of the following doses may be used: a dose of at least about 50 mg/kg body weight; at least about 20 mg/kg body weight; at least about 10 mg/kg body weight; at least about 5 mg/kg body weight; at least about 3 mg/kg body weight; at least about 2 mg/kg body weight; at least about 1 mg/kg body weight; at least about 750 μg/kg body weight; at least about 500 μg/kg body weight; at least about 250 ug/kg body weight; at least about 100 μg/kg body weight; at least about 50 μg/kg body weight; at least about 10 ug/kg body weight; at least about 1 μg/kg body weight, or more, is administered. Empirical considerations, such as the half-life, generally will contribute to determination of the dosage. For repeated administrations over several days or longer, depending on the condition, the treatment is sustained until a desired suppression of disease symptoms occurs or until sufficient therapeutic levels are achieved. For example, dosing from one to five times a week is contemplated. Other dosing regimens include a regimen of up to 1 time per day, 1 to 5 times per week, or less frequently. In some embodiments, the NT-4/5 polypeptide are administered about once per week, about 1 to 4 times per month. Intermittent dosing regime with staggered dosages spaced by 2 days up to 7 days or even 14 days may be used. In some embodiments, treatment may start with a daily dosing and later change to weekly even monthly dosing. The progress of this therapy is easily monitored by conventional techniques and assays.

In some individuals, more than one dose may be required. Frequency of administration may be determined and adjusted over the course of therapy. For example, frequency of administration may be determined or adjusted based on the type and severity of the disease to be treated (such as the obesity and non-insulin dependent diabetes mellitus), whether the agent is administered for preventive or therapeutic purposes, previous therapy, the patient's clinical history and response to the agent, and the discretion of the attending physician. Typically the clinician will administer NT-4/5 polypeptide until a dosage is reached that achieves the desired result. In some cases, sustained continuous release formulations of NT-4/5 polypeptide may be appropriate. Various formulations and devices for achieving sustained release are known in the art. For example, NT-4/5 polypeptide may be administered through a mechanical pump or embedded in a matrix bed for sustained or slow release.

In one embodiment, dosages for NT-4/5 polypeptide may be determined empirically in individuals who have been given one or more administration(s). Individuals are given incremental dosages of NT-4/5 polypeptide. To assess efficacy of NT-4/5 polypeptide, markers of the disease state can be monitored. Examples of such markers are blood glucose level, blood lipid level (e.g., triglyceride or cholesterol), insulin resistance, body weight, and food intake. It will be apparent to one of skill in the art that the dosage will vary depending on the individual, the stage of the disease (such as obesity and diabetes), and the past and concurrent treatments being used.

Administration of NT-4/5 polypeptide in accordance with the method in the present invention can be continuous or intermittent, depending, for example, upon the recipient's physiological condition, whether the purpose of the administration is therapeutic or prophylactic, and other factors known to skilled practitioners. The administration of an NT-4/5 polypeptide may be essentially continuous over a preselected period of time or may be in a series of spaced doses.

Other formulations include suitable delivery forms known in the art including, but not limited to, carriers such as liposomes. See, for example, Mahato et al. (1997) Pharm. Res. 14:853-859. Liposomal preparations include, but are not limited to, cytofectins, multilamellar vesicles and unilamellar vesicles.

An expression vector can be used to direct expression of NT-4/5 polypeptide in the brain, for example hypothalamus. One skilled in the art is familiar with administration of expression vectors to obtain expression of an exogenous protein in vivo. See, e.g., U.S. Pat. Nos. 6,436,908; 6,413,942; and 6,376,471. Administration of expression vectors includes local or systemic administration, including injection, oral administration, particle gun or catheterized administration, and topical administration. In another embodiment, the expression vector is administered directly to skeletal muscle or subdermal space.

Targeted delivery of therapeutic compositions containing an expression vector, or subgenomic polynucleotides can also be used. Receptor-mediated DNA delivery techniques are described in, for example, Findeis et al., Trends Biotechnol. (1993) 11:202; Chiou et al., Gene Therapeutics Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. The therapeutic polynucleotides and polypeptides can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) δ: 148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0524968. Additional approaches are described in Philip, Mol. Cell Biol. (1994) 14:2411, and in Woffendin, Proc. Natl. Acad. Sci. (1994) 91:1581.

Assessment of disease is performed using standard methods known in the arts, for example, by monitoring appropriate marker(s), such as blood glucose, lipids, insulin levels, and body weight.

### Compositions and Methods of Making the Compositions

The compositions used in the methods of the invention comprise an NT-4/5 polypeptide or polynucleotide encoding an NT-4/5 polypeptide. As used herein, “NT-4/5 polypeptide” includes naturally-occurring mature protein (interchangeably termed “NT-4/5”) such as mature human NT-4/5 shown in Table 1 below and FIG. 1 in U.S. Pat. Appl. Pub. No. 20030203383 and naturally occurring amino acid sequence variants of NT-4/5; amino acid sequence variants of NT-4/5; peptide fragments of mature NT-4/5 (such as human) and said amino acid sequence variants; and modified forms of mature NT-4/5 and said amino acid sequence variants and peptide fragments wherein the polypeptide or peptide has been covalently modified by substitution with a moiety other than a naturally occurring amino acid, as long as the amino acid sequence variant, peptide fragment, and the modified form thereof show one or more biological activities of naturally occurring mature NT-4/5 protein. The NT-4/5 polypeptides also include fusion proteins and conjugates comprising any of the NT-4/5 polypeptide embodiments described herein, e.g., an NT-4/5 polypeptide conjugated or fused to a half life extending moiety, such as a PEG or a peptide. The amino acid sequence variants, peptide fragments (including fragments of variants), or modified forms thereof under consideration do not include NGF, BDNF, or NT-3 of any animal species. Variants, peptide fragments, and modified forms of naturally occurring NT-4/5 are described in U.S. Pat. Appl. Pub. Nos. 20030203383; 20020045576; U.S. Pat. Nos. 5,702,906; 6,506,728; 6,566,091; 5,830,858; which are incorporated by reference in their entirety. NT-4/5 polypeptides include any one or more embodiments described herein. For example, NT-4/5 polypeptide comprises a naturally occurring sequence with one or more amino acid insertion, deletion, or substitution.

In some embodiments, the NT-4/5 polypeptide is a mammalian NT-4/5 polypeptide which may be a naturally occurring mammalian NT-4/5, or NT-4/5 polypeptide derived from a naturally occurring mammalian NT-4/5 and having a sequence that does not match any part of a naturally occurring non-mammalian NT-4/5. In some embodiments, the NT-4/5 polypeptide is a human NT-4/5 polypeptide which may be a naturally occurring human NT-4/5, or NT-4/5 polypeptide derived from a naturally occurring human NT-4/5 and having a sequence that does not match any part of a naturally occurring non-human NT-4/5.

TABLE 1

Amino acid sequence of mature human NT-4/5
and the human nucleotide sequence encoding
the mature human NT-4/5

Amino acid sequence (SEQ ID NO:1):
GVSETAPASRRGELAVCDAVSGWVTDRRTAVDLRGREVEVLGEVPAAGGS
PLRQYFFETRCKADNAEEGGPGAGGGGCRGVDRRHWVSECKAKQSYVRAL
TADAQGRVGWRWIRIDTACVCTLLSRTGRA
Nucleotide sequence (SEQ ID NO:2)
GGGGTGAGCGAAACTGCACCAGCGAGTCGTCGGGGTGAGCTGGCTGTGTG
CGATGCAGTCAGTGGCTGGGTGACAGACCGCCGGACCGCTGTGGACTTGC
GTGGGCGCGAGGTGGAGGTGTTGGGCGAGGTGCCTGCAGCTGGCGGCAGT
CCCCTCCGCCAGTACTTCTTTGAAACCCGCTGCAAGGCTGATAACGCTGA
GGAAGGTGGCCCGGGGGCAGGTGGAGGGGGCTGCCGGGGAGTGGACAGGA
GGCACTGGGTATCTGAGTGCAAGGCCAAGCAGTCCTATGTGCGGGCATTG
ACCGCTGATGCCCAGGGCCGTGTGGGCTGGCGATGGATTCGAATTGACAC
TGCCTGCGTCTGCACACTCCTCAGCCGGACTGGCCGGGCCTGAG

NT-4/5 polypeptides, including variants, peptide fragments, modified forms of NT-4/5 polypeptides (including naturally occurring NT-4/5), fusion protein and conjugate of the invention are characterized by any (one or more) of the following characteristics: (a) ability to bind to and activate TrkB receptor; (b) ability to activate one or more downstream pathways mediated by the TrkB signaling function; (c) treat, prevent, or ameliorate one or more symptoms of non-insulin dependent diabetes mellitus; and (d) treat, prevent, or ameliorate one or more symptoms of obesity. Thus all NT-4/5 polypeptides (including variants, fragments, and modified forms) are functional as described above.

Biological activity of variants may be tested in vitro and in vivo using methods known in the art and methods described in Examples. NT-4/5 polypeptides may have an enhanced activity or reduced activity as compared to a naturally occurring NT-4/5 protein. In some embodiments, functionally equivalent variants have at least about any of 50%, 60%, 70%, 75%, 80%, 85%, 90%, or 95% of activity as compared to the native NT-4/5 protein from which the NT-4/5 polypeptide is derived with respect to one or more of the biological assays described above (or known in the art). In some embodiments, functionally equivalent variants have an EC₅₀ (half of the maximal effective concentration) of less than about any of 0.01 nM, 0.1 nM, 1 nM, 10 nM, or 100 nM in TrkB receptor activation in vitro (e.g., assays described in Examples 6 and 7).

Amino acid sequence variants of NT-4/5 include polypeptides having an amino acid sequence which differs from naturally occurring NT-4/5 by virtue of the insertion, deletion, and/or substitution of one or more amino acid residues within the sequence of naturally occurring NT-4/5 (for example, mature human NT-4 shown in Table 1). Amino acid sequence variants generally will be at least about 65%, 70%, 75%, 80%, 85%, 90%, 95%, 96%, 97%, 98%, or 99% identical to any naturally occurring NT-4/5 (such as mature human NT-4/5 shown in SEQ ID NO:1). In some embodiments, the variant is at least about 70% identical to the amino acid sequence of SEQ ID NO:1. In some embodiments, the variant is at least about 85% identical to the amino acid sequence of SEQ ID NO:1. In some embodiments, the variant is at least about 90% identical to the amino acid sequence of SEQ ID NO:1. In some embodiments, the variant is at least about 95% identical to the amino acid sequence of SEQ ID NO:1.

Amino acid sequence variants of NT-4/5 can be generated by making predetermined mutations in a previously isolated NT-4/5 DNA. Amino acid variants may be designed and generated based on crystal structure of NT-4/5 and TrkB receptor. Banfield et al., Structure 9: 1191-9 (2001) For example, amino acids that are not directly involved in interaction between monomers of NT-4/5 and between NT-4/5 and the TrkB receptor may be mutated, for example, to introduce PEG attaching site. Methods known in the art may be used to design variants of NT-4/5 polypeptide that have enhanced or reduced one or more biological activities as compared to the naturally occurring NT-4/5 protein.

There are two principal variables to consider in making such predetermined mutations: the location of the mutation site and the nature of the mutation. In general, the location and nature of the mutation chosen generally depends upon the NT-4/5 characteristic to be modified. For example, candidate NT-4/5 antagonists or super agonists initially can be selected by locating amino acid residues that are identical or highly conserved among NGF, BDNF, NT-3, and NT-4. Those residues can then be modified in series, e.g., by (1) substituting first with conservative choices and then with more radical selections depending upon the results achieved, (2) deleting the target residue, or (3) inserting residues of the same or different class adjacent to the located site, or combinations of options 1-3.

One helpful technique is called “ala scanning”. Here, an amino acid residue or group of target residues are identified and substituted by alanine or polyalanine. Those domains demonstrating functional sensitivity to the alanine substitutions then are refined by introducing further or other variants at or for the sites of alanine substitution.

Obviously, such variations which, for example, convert NT-4/5 into NGF, BDNF, or NT-3 are not included within the scope of this invention. Thus, while the site for introducing an amino acid sequence variation is predetermined, the nature of the mutation per se need not be predetermined. For example, to optimize the performance of a mutation at a given site, ala scanning or random mutagenesis is conducted at the target codon or region and the expressed NT-4/5 variants are screened for the optimal desired activity.

Amino acid sequence deletions generally range from about 1 to 30 residues, more preferably about 1 to 10 residues, and typically are contiguous. Deletions may be introduced into regions of low homology among BDNF, NGF, NT-3, and NT-4/5 to modify the activity of NT-4/5. Deletions from NT-4/5 in areas of substantial homology with BDNF, NT-3, and NGF may be more likely to modify the biological activity of NT-4/5 more significantly. The number of consecutive deletions may be selected so as to preserve the tertiary structure of NT-4/5 in the affected domain, e.g., beta-pleated sheet or alpha helix.

Amino acid sequence insertions include amino- and/or carboxyl-terminal fusions ranging in length from one residue to polypeptides containing a thousand or more residues, as well as intrasequence insertions of single or multiple amino acid residues. Intrasequence insertions (i.e., insertions within the mature NT-4/5 sequence) may range generally from about 1 to 10 residues, more preferably 1 to 5, most preferably 1 to 3. An example of a terminal insertion includes fusion of a heterologous N-terminal signal sequence to the N-terminus of the NT-4/5 molecule to facilitate the secretion of mature NT-4/5 from recombinant host. Such signals generally will be homologous to the intended host cell and include STII or lpp for E. coli, alpha factor for yeast, and viral signals such as herpes gD for mammalian cells. Other insertions include the fusion of a polypeptide to the N- or C-termini of NT-4/5.

The third group of variants includes those in which at least one amino acid residue in NT-4/5, and preferably only one, has been removed and a different residue inserted in its place. An example is the replacement of arginine and lysine by other amino acids to render the NT-4/5 resistant to proteolysis by serine proteases, thereby creating a variant of NT-4/5 that is more stable. The sites of greatest interest for substitutional mutagenesis include sites where the amino acids found in BDNF, NGF, NT-3, and NT-4 are substantially different in terms of side chain bulk, charge or hydrophobicity, but where there also is a high degree of homology at the selected site within various animal analogues of NGF, NT-3, and BDNF (e.g. among all the animal NGFs, all the animal NT-3, and all the BDNFs). This analysis will highlight residues that may be involved in the differentiation of activity of the trophic factors, and therefore, variants at these sites may affect such activities. Examples of such sites in mature human NT-4/5, numbered from the N-terminal end, and exemplary substitutions include G77 to K, H, Q or R and R84 to E, F, P, Y or W of NT-4/5 of SEQ ID NO:1, respectively. Other sites of interest are those in which the residues are identical among all animal species BDNF, NGF, NT-3, and NT-4/5, this degree of conformation suggesting importance in achieving biological activity common to all four factors.

For example, substitution of one or more amino acids includes conservative substitutions. Methods of making conservative substitutions are known in the art. For example, ala (A) may be substituted by val, leu, ile, preferably by val; arg (R) may be substituted by lys, gln, asn, preferably by lys; asn (N) may be substituted by gln, his, lys, arg, preferably by gln; asp (D) may be substituted by glu; cys (C) may be substituted by ser; gln (Q) may be substituted by asn; glu (E) may be substituted by asp; gly (G) may be substituted by pro; his (H) may be substituted by asn, gln, lys, arg; preferably by arg; ile (I) may be substituted by leu, val, met, ala, phe, norleucine, preferably by leu; leu (L) may be substituted by norleucine, ile, val, met; ala; phe, preferably by ile; lys (K) may be substituted by arg; gln, asn, preferably by arg; met (M) may be substituted by leu; phe; ile, preferably by leu; phe (F) may be substituted by leu, val, ile, ala, preferably by leu; pro (P) may be substituted by gly; ser (S) may be substituted by thr; thr (T) may be substituted by ser; trp (W) may be substituted by tyr; tyr (Y) may be substituted by trp, phe, thr, ser, preferably by phe; val (V) may be substituted by ile; leu; met; phe, ala; norleucine, preferably by leu.

Sites particularly suited for conservative substitutions include, numbered from the N-terminus of the mature human NT-4 (SEQ ID NO:1), R11, G12, E13, V16, D18, W23, V24, D26, V40, L41, Q54, Y55, F56, E58, T59, G77, R79, G80, H85, W86, A99, L100, T101, W110, R111, W112, I113, R114, I115, D116, and A118. Cysteine residues not involved in maintaining the proper conformation of NT-4/5 also may be substituted, generally with serine, in order to improve the oxidative stability of the molecule and prevent aberrant crosslinking. Sites other than those set forth in this paragraph are suitable for deletional or insertional studies generally described above.

Substantial modifications in function may be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side chain properties (some of these may fall into several functional groups):

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another.

Examples of NT-4 variants include: polypeptide of SEQ ID NO:1 with mutation of E67 to S or T (this adds an N-linked glycosylation site); polypeptide from amino acid residue R83 to Q94, G1 to C61, G1 to C17, C17 to C61, C17 to C78, C17 to C90, C17 to C119, C17 to C121, R11 to R27, R11 to R34, R34 to R53, C61 to C78, R53 to C61, C61 to C119, C61 to C78, C78 to C119, C61 to C90, R60 to C78, K62 to C119, K62 to K91, R79 to R98, R83 to K93, T101 to R11, G1 to C121 of SEQ ID NO:1; polypeptide comprises V40-C121 of SEQ ID NO:1, for example, V40-C121 of SEQ ID NO:1 fused to a polypeptide at the N-terminal and/or C-terminal; polypeptide comprises SEQ ID NO:1 with deletion of C78, C61, Q54-T59, R60-D82, H85-S88, W86-T111 (deletions of the indicated span of residues, inclusive); SEQ ID NO:1 with mutation from R53 to H, from K91 to H, from V108 to F, from R84 to Q, H, N, T, Y or W, and from D116 to E, N, Q, Y, S or T. Also included is NT-4/5 (SEQ ID NO:1) wherein position 70 is substituted with an amino acid residue other than G, E, D or P; position 71 with other than A, P or M; and/or position 83 with other than R, D, S or K; as well as cyclized NT-4 fragments.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Amino acid sequence variants of NT-4/5 may be naturally occurring or may be prepared synthetically, such as by introducing appropriate nucleotide changes into a previously isolated NT-4/5 DNA, or by in vitro synthesis of the desired variant polypeptide. As indicated above, such variants may comprise deletions from, or insertions or substitutions of, one or more amino acid residues within the amino acid sequence of mature NT-4/5 (e.g., sequence shown in Table 1). Any combination of deletion, insertion, and substitution is made to arrive at an amino acid sequence variant of NT-4/5, provided that the resulting variant polypeptide possesses a desired characteristic. The amino acid changes also may result in further modifications of NT-4/5 upon expression in recombinant hosts, e.g. introducing or moving sites of glycosylation, or introducing membrane anchor sequences (in accordance with PCT WO 89/01041 published Feb. 9, 1989).

In some embodiments, NT-4/5 polypeptide comprises an amino acid sequence encoded by a nucleic acid that hybridizes under stringent conditions to a nucleic acid sequence (e.g., SEQ ID NO:2) encoding mature human NT-4/5.

Variants polynucleotides may also, or alternatively, be substantially homologous to a native gene, or a portion or complement thereof. Such polynucleotide variants are capable of hybridizing under moderately stringent conditions to a naturally occurring DNA sequence encoding a the polypeptide (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Another exemplary stringent condition hybridization in 50% formamide, 5×SSC, 0.1% sodium dodecyl sulfate, 0.1% sodium pyrophosphate, 50 mM sodium phosphate pH 6.8, 2×Denhardt's solution, and 10% dextran sulfate at 42° C., followed by a wash in 0.1×SSC and 0.1% SDS at 42° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Variants of NT-4/5 used in the methods of the invention also include fusion proteins comprising the amino acid sequence of NT-4/5 (e.g., human NT-4/5 shown in Table 1) or a functional peptide fragment thereof. Biologically active NT-4/5 polypeptides can be fused with sequences, such as sequences that enhance immunological reactivity, facilitate the coupling of the polypeptide to a support or a carrier, or facilitate refolding and/or purification (e.g., sequences encoding epitopes such as Myc, HA derived from influenza virus hemagglutinin, His-6, FLAG). These sequences may be fused to NT-4/5 polypeptide at the N-terminal end or at the C-terminal end. In addition, the protein or polynucleotide can be fused to other or polypeptides which increase its function, or specify its localization in the cell, such as a secretion sequence. Methods for producing recombinant fusion proteins described above are known in the art. The recombinant fusion protein can be produced, refolded and isolated by methods well known in the art.

NT-4/5 polypeptides described herein may be modified to increase their half lives in an individual. For example, NT-4/5 polypeptide may be pegylated to reduce systemic clearance with minimal loss of biological activity. The invention also provides compositions (including pharmaceutical compositions) comprising an NT-4/5 polypeptide linked to a PEG molecule. In some embodiments, the PEG molecule is linked to the NT-4/5 polypeptide through a reversible linkage. The half life of a pegylated NT-4/5 polypeptide may be extended by more than about any of 2-fold, 5-fold, 10-fold, 15-fold, 20-fold, and 30-fold of the half life of the non-pegylated NT-4/5 polypeptide.

Polyethylene glycol polymers (PEG) may be linked to various functional groups of the NT-4/5 polypeptide using methods known in the art. See, e.g., Roberts et al., Advanced Drug Delivery Reviews 54:459-476 (2002); Sakane et al. Pharm. Res. 14:1085-91 (1997). PEG may be linked to the following functional groups on the polypeptide: amino groups, carboxyl groups, modified or natural N-termini, amine groups, and thiol groups. In some embodiments, one or more surface amino acid residues are modified with PEG molecules. PEG molecules may be of various sizes (e.g., ranging from about 2 to 40 KDa). PEG molecules linked to NT-4/5 polypeptide may have a molecular weight about any of 2000, 10,000, 15,000, 20,000, 25,000, 30,000, 35,000, 40,000 Da. PEG molecule may be a single or branched chain. To link PEG to NT-4/5 polypeptide, a derivative of the PEG having a functional group at one or both termini may be used. The functional group is chosen based on the type of available reactive group on NT-4/5 polypeptide. Methods of linking derivatives to polypeptides are known in the art. Roberts et al., Advanced Drug Delivery Reviews 54:459-476 (2002). The linkage between the NT-4/5 polypeptide and the PEG may also be such that it can be cleaved or naturally degrades (reversible or degradable linkage) in an individual which may improve the half-life but minimize loss of activity. PEG linking site on NT-4/5 polypeptide may also be created by mutating surface residues to an amino acid residue having a PEG reactive group, such as, a cysteine. For example, the following amino acids of human NT-4/5 (SEQ ID NO:1) may be mutated for PEG attachment: G1, V2, S3, E4, T5, S9, R10, T25, D26, R28, T29, V31, E37, E39, L41, E43, A46, A47, G48, G49, S50, R53, D64, N65, A66, E67, E68, G69, D82, R83, R84, H85, A104, Q105, G106, R107, V108, S125, and T127. These may be applied to the corresponding residues in other species.

Several pegylated NT-4/5 have been generated and are shown in Examples 6 and 7. Serine residue at position 50 of the mature human NT-4/5 may be changed to cysteine to generate NT4-S50C which is then pegylated, wherein the PEG is linked to the cysteine at position 50. One example of an N-terminal specific attachment for PEG is to mutate the residue at position 1 to a serine or threonine, then followed with pegylation, wherein the PEG is linked to the serine at position 1.

NT-4/5 polypeptide may also be modified to increase their efficiency of being transferred through blood brain barrier to the brain after peripheral administration. For example, NT-4/5 polypeptide may be conjugated or linked to an anti-transferrin receptor antibody. See, Pardridge W M, Pharmacol. Toxicol. 71:3-10 (1992). NT-4/5 polypeptide may be conjugated to an anti-transferrin receptor antibody via avidin/biotin interaction or covalently coupled to an anti-transferrin receptor antibody (such as the OX26 antibody). See, Kang et al., J. Pharmacol. Exp. Ther. 269:344-50 (1994); Yoshikawa, et al., J. Pharmacol. Exp. Ther. 263:897-903 (1992); Pardridge W M, Pharmacol. Toxicol. 71:3-10 (1992). Pegylated NT-4/5 polypeptides may also be linked to an anti-transferrin receptor antibody. See Wu et al., Proc. Natl. Acad. Sci. U.S.A. 96:254-9 (1999). Delivery of NT-4/5 polypeptides to the brain may also be achieved using immunoliposomes (antibody-directed liposomes). Liposomes carrying NT-4/5 polypeptides may be conjugated to PEG and then coupled to an anti-transferrin receptor antibody. Huwyler et al. Proc. Natl. Acad. Sci. U.S.A. 93:14164-9 (1996). Other antibodies specifically identified for their ability to cross the blood brain barrier may also used for any of the above formulations. Muruganandam et al., FASEB J. 16:240-2 (2002).

NT-4/5 polypeptide can be produced by recombinant means, that is, by expression of nucleic acid encoding the NT-4/5 polypeptide. In recombinant cell culture, and, optionally, purification of the variant polypeptide from the cell culture, for example, by bioassay of the variant's activity or by adsorption on an immunoaffinity column comprising rabbit anti-NT-4/5 polyclonal antibodies (which will bind to at least one immune epitope of the variant which is also present in native NT-4/5). Small peptide fragments, on the order of 40 residues or less, are conveniently made by in vitro methods.

DNA encoding NT-4/5 polypeptide may be cloned into an expression vector for expressing the protein in a host cell. Examples of nucleic acids encoding NT-4/5 polypeptide are described in U.S. Pat. Appl. Pub. No. 2003/0203383. The DNA encoding NT-4/5 polypeptide in its mature form may be linked at its amino terminus to a secretion signal. This secretion signal preferably is the NT-4/5 presequence that normally directs the secretion of NT-4/5 from human cells in vivo. However, suitable secretion signals also include signals from other animal NT-4/5, signals from NGF, NT-2, or NT-3, viral signals, or signals from secreted polypeptides of the same or related species. Any host cell (such as E. coli) may be used for expressing the protein or polypeptide.

NT-4/5 polypeptide expressed may be purified. NT-4/5 polypeptide may be recovered from the culture medium as a secreted protein, although it also may be recovered from host cell lysates when directly expressed without a secretory signal. Protein purification methods known in the art may be used. Methods of producing NT-4/5 polypeptide and purifying the expressed NT-4/5 polypeptide are described in U.S. Pat. Appl. Pub. No. 2003/0203383, and U.S. Pat. No. 6,184,360. NT-4/5 polypeptide can be expressed in E. coli and refolded according to methods known in the art. Mature human NT-4/5 may also be obtained commercially (for example, from R&D Systems, Sigma and Upstate).

The invention also provides compositions comprising an NT-4/5 polypeptide and an antibody that specifically binds to the NT-4/5 polypeptide. The antibody and the NT-4/5 polypeptide may be present in a predetermined ratio. Thus, in some embodiments, the ratio by weight of the antibody to the NT-4/5 polypeptide may be approximately 1 to 1. In some embodiments, this ratio may be between about 0.001 to about 1 and about 1 to about 1000, between about 0.01 to about 1 and about 1 to about 100, or between about 0.1 to about 1 and about 1 to about 10. Other ratios are contemplated. In some embodiments, the antibody and the NT-4/5 polypeptide are present in a single dosage unit. The binding affinity of the antibody to the NT-4/5 polypeptide may be about 2 pM to about 500 nM. The binding affinity may be less than about any of 500 nM, 200 nM, 100 nM, 10 nM, 5 nM, 1 nM, 900 pM, 500 pM, 300 pM, 150 pM, 100 pM, 50 pM, 25 pM, and 10 pM.

The composition used in the present invention can further comprise pharmaceutically acceptable carriers, excipients, or stabilizers (Remington: The Science and practice of Pharmacy 20th Ed. (2000) Lippincott Williams and Wilkins, Ed. K. E. Hoover.), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

NT-4/5 polypeptides described herein can be formulated for sustained-release. Suitable examples of sustained-release preparations include semipermeable matrices of solid hydrophobic polymers containing NT-4/5 polypeptide, which matrices are in the form of shaped articles, e.g. films, or microcapsules. Examples of sustained-release matrices include polyesters, hydrogels (for example, poly(2-hydroxyethyl-methacrylate), or ‘poly(v nylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.n Another example of sustained release drug-delivery system that can be used is the ATRIGELL™ made by Atrix Laboratories. See, for example U.S. Pat. No. 6,565,874. The ATRIGELL™ drug delivery system consists of biodegradable polymers, similar to those used in biodegradable sutures, dissolved in biocompatible carriers. NT-4/5 polypeptides may be blended into this liquid delivery system at the time of manufacturing or, depending upon the product, may be added later by the physician at the time of use. When the liquid product is injected subcutaneously or intramuscularly through a small gauge needle or placed into accessible tissue sites through a cannula, displacement of the carrier with water in the tissue fluids causes the polymer to precipitate to form a solid film or implant. NT-4/5 polypeptides encapsulated within the implant are then released in a controlled manner as the polymer matrix biodegrades with time. Depending upon the patient's medical needs, the Atrigel system can deliver proteins over a period ranging from days to months. Injectable sustained release systems, such as ProLease®, Medisorb®, manufactured by Alkermes may also be used.

In some embodiments, the invention provides compositions (described herein) for use in any of the methods described herein, whether in the context of use as a medicament and/or use for manufacture of a medicament.

### Kits Comprising NT-4/5 Polypeptide for Therapeutic Purposes

The invention also provides kits for use in the instant methods. Kits of the invention include one or more containers comprising purified NT-4/5 polypeptide (including naturally occurring NT-4/5) and instructions for use in accordance with any of the methods of the invention described herein. Generally, these instructions comprise a description of administration of NT-4/5 polypeptide to treat a disease, such as obesity and non-insulin dependent diabetes mellitus, according to any of the methods described herein. The kit may further comprise a description of selecting an individual suitable for treatment based on identifying whether that individual has the disease and the stage of the disease (such as obesity and non-insulin dependent diabetes mellitus).

In some embodiments, the kit further comprises an antibody that specifically binds to the NT-4/5 polypeptide and instructions for administering the NT-4/5 polypeptide in conjunction with the antibody.

The instructions relating to the use of NT-4/5 polypeptide generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating a disease described herein (such as obesity and non-insulin dependent diabetes mellitus). Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is NT-4/5 polypeptide. The container may further comprise a second pharmaceutically active agent.

Kits may optionally provide additional components such as buffers and interpretive information. Normally, the kit comprises a container and a label or package insert(s) on or associated with the container.

The following examples are provided to illustrate, but not to limit, the invention.

### EXAMPLES

For all examples described below, human NT-4/5 was used for all the experiments. Thus, in the examples below, NT-4/5 refers to human NT-4/5.

### Example 1

### Effect of NT-4/5 on Carbohydrate and Lipid Metabolism in db/db Mice

### A. Experimental Protocol

Test animals: 24 female 10-12 weeks old db/db mice (C57BL/Ks J Rj-db/db, Janvier, France), weighing in the target range of 40-50 g, were used in this study. These mice were housed in a temperature (19.5-24.5° C.) and relative humidity (45-65%) controlled room with a 12-hour light/dark cycle, with ad libitum access to filtered tap-water and irradiated pelleted laboratory chow (SAFE, France) throughout the study. Upon receipt at animal facilities, they were housed 4 per cage covered with filtered caps and at least a 5-day acclimation period was observed.

Administration of NT-4/5 and reference substances: Rosiglitazone (3 mg/kg, Sequoia Research Products Ltd., UK), a thiazolidinedione compound for the therapy of type 2 diabetes, was used in the present experiment as a reference. Phosphate buffer saline was used as vehicle in this experiment. NT-4/5 was obtained from Genentech.

The mice were divided into three groups of 8 mice each (groups 1-3). Each group was treated with vehicle (PBS) subcutaneously (s.c.), 3 mg/kg Rosiglitazone orally (p.o.), or 20 mg/kg NT-4/5 subcutaneously (s.c.). From day 1 to day 5 (T1-T5) and then from day 8 to day 12 (T8-T12), the mice in groups 2 and 3 were dosed once daily for 5 consecutive days, with a volume of 10 ml/kg.

Collection of samples for analysis: One day before beginning the treatment (T0), non fasted db/db mice were weighed and blood samples were collected through the retro-orbital plexus (about 300 μL/mouse) under isoflurane anesthesia. Similarly, at day 5 (T5), day 12 (T12), day 19 (T19) and day 26 (T26), two hours after the last administration, blood samples were collected through the retro-orbital plexus under isoflurane anesthesia in non fasted mice. Blood samples were subjected to HbA1c determination before kept at room temperature for 5 to 10 minutes to form a spontaneous clot, then put on ice until they were centrifuged at 3500×g for 10-15 minutes at 4° C. using a 2K15 model centrifuge (Sigma, France). An aliquot of serum was used for measuring glucose levels, the remaining was frozen until use.

Analysis of blood samples in db/db mice: Pre- and post dose serum levels of glucose, HbA1c, cholesterol and triglycerides, insulin, and non esterified fatty acids (NEFA), as well as aspirate amino-transferase (AST) and alanine amino-transferase activities (ALT) activities, were determined at different time points, as described further herein. At each time point and for each parameter, a mean % of the effect was calculated according to the following formula: ((T final−T initial)/T initial)*100. Statistical analysis was consistent in a one-way analysis of variance followed by multiple comparisons versus the vehicle group (Dunnett's test) on final values. In case the equal variance test fails, a Kruskall-Wallis one-way analysis of variance on ranks was conducted. A difference was considered significant for p<0.05.

### B. Results

1. Blood Glucose Controlling Activity of NT 4/5 in db/db Mice

Blood glucose levels of treated mice were determined using a Glucose kit (ref. 442640, Beckman Coulter, France) and a Synchron CX-4 analyzer (Beckman Coulter, France). Table 2 summarizes the results of the analysis. The results are also graphically presented in FIG. 1.

TABLE 2
Effect of NT-4/5 on blood glucose levels (mM) in db/db mice

| | | | % effect | | % effect | | % effect | | % effect |
|---|---|---|---|---|---|---|---|---|---|
| | T0 | T5 | T5-T0 | T12 | T12-T0 | T19 | T19-T0 | T26 | T26-T0 |
| Day | 0 | 5 | | 12 | | 19 | | 26 | |
| Vehicle | 37.39 | 29.74 | −19.2 | 34.89 | −5.8 | 43.46 | 17.0 | 46.83 | 25.5 |
| (s.c.) | 1.87 | 3.13 | 10.1 | 4.38 | 13.5 | 2.14 | 11.9 | 1.58 | 10.4 |
| Rosiglitazone | 37.56 | 22.44 | −40.5 | 22.24 | −40.9 | 33.74 | −9.0 | 43.60 | 17.5 |
| (3 mg/kg p.o.) | 1.74 | 2.80 | 6.8 | 2.57* | 5.8 | 1.85* | 6.0 | 2.44 | 7.6 |
| NT-4/5 | 36.95 | 15.78 | −56.4 | 13.56 | −62.6 | 12.65 | −65.2 | 22.50 | −37.9 |
| (20 mg/kg s.c) | 1.99 | 1.18* | 4.2 | 1.58* | 5.2 | 1.48* | 5.0 | 1.63* | 6.7 |

Values are expressed as mean ± SEM
Statistics: One-way analysis of variance (ANOVA) followed by a Dunnett's test.
*p < 0.05 as compared to vehicle

The blood glucose level of vehicle-treated mice remained high throughout the study. In mice treated with Rosiglitazone, the blood glucose level decreased to about 22.44 mM at T5, stayed low until T12, and went up dramatically afterwards. The blood glucose level in mice treated with NT 4/5 decreased to 15.78 mM at T5, decreased to 13.56 mM at T12, and further decreased to 12.65 mM at T19, reaching almost the normal level of blood glucose (about 10.95±0.41 mM, determined by measuring 8 normal healthy C57BL/6J mice). The level of blood glucose in NT 4/5 treated mice remained at least 50% lower than that of vehicle treated mice throughout the rest of the study.

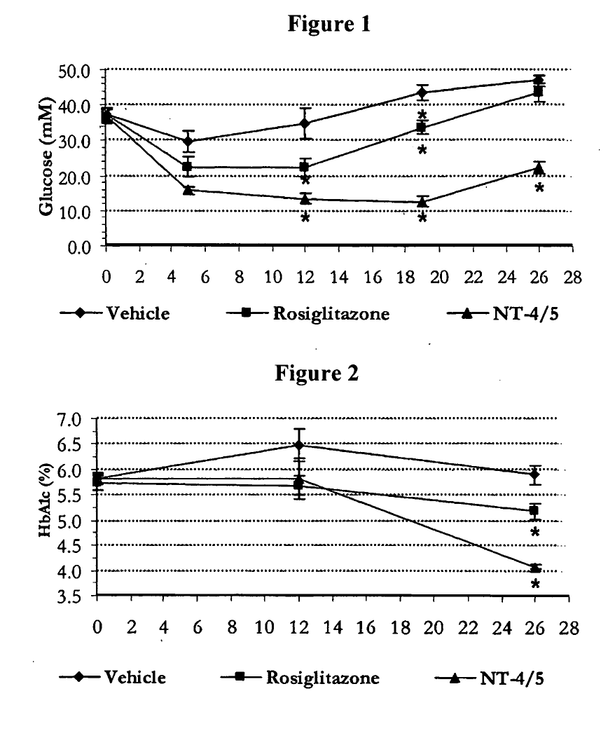

HbA1c levels were determined using an HbA1c kit (ref. 650262, Beckman Coulter, France) and a Synchron CX4 synchronizer. HbA1c is an indicator for blood glucose control over the last 2-3 months. The normal level of HbA1c in C57BL/6J mouse (determined by measuring 8 normal healthy C57BL/6J mice) was about 3.5%. Table 3 summarizes the results of the analysis. The results are also graphically presented in FIG. 2.

TABLE 3
Effect of NT-4/5 on HbA1c (%) in db/db mice

| | | | % effect | | % effect |
|---|---|---|---|---|---|
| | T0 | T12 | T12-T0 | T26 | T26-T0 |
| Day | 0 | 12 | | 26 | |
| Vehicle | 5.84 | 6.49 | 11.5 | 5.90 | 1.1 |
| (s.c.) | 0.10 | 0.31 | 6.0 | 0.18 | 4.2 |
| Rosiglitazone | 5.74 | 5.69 | −1.0 | 5.18 | −9.6 |
| (3 mg/kg p.o.) | 0.15 | 0.19 | 1.7 | 0.17* | 3.2 |
| NT-4/5 | 5.81 | 5.82 | −0.2 | 4.08 | −30.4 |
| (20 mg/kg s.c.) | 0.11 | 0.41 | 8.4 | 0.05* | 1.1 |

Values are expressed as mean ± SEM
Statistics: One-way analysis of variance (ANOVA)
*p < 0.05 as compared to vehicle

The level of HbA1c in both Rosiglitazone and NT 4/5 treated mice decreased only slightly at T12 (1% and 0.2% respectively). However, the HbA1c level was significantly suppressed at T26. In Rosiglitazone treated mice, the HbA1c level decreased 9.6%. In NT-4/5 treated mice, the HbA1c level at T26 decreased 30.4% as compared to T0.

These results demonstrate that both short-term and long term blood glucose levels have been significantly reduced in NT-4/5 treated mouse.

2. Blood Lipid Controlling Activity of NT-4/5 in db/db Mice

The blood triglyceride level and the total blood cholesterol level were determined with a triglyceride assay kit (ref 445850, Beckman Coutler, France) and a total cholesterol kit (ref 467825, Beckman Coutler, France), respectively, using a Synchron CX4 analyzer. The results of the experiment were shown in Tables 4 and 5.

TABLE 4
Effect of NT-4/5 on triglycerides levels (mM)

| | | | % effect | | % effect | | % effect | | % effect |
|---|---|---|---|---|---|---|---|---|---|
| | T0 | T5 | T5-T0 | T12 | T12-T0 | T19 | T19-T0 | T26 | T26-T0 |
| Days | 0 | 5 | | 12 | | 19 | | 26 | |
| Vehicle | 1.42 | 1.94 | 48.4 | 1.87 | 42.6 | 1.05 | −16.1 | 2.63 | 111.1 |
| (s.c.) | 0.21 | 0.18 | 16.0 | 0.24 | 18.9 | 0.09 | 8.6 | 0.26 | 26.5 |
| Rosiglitazone | 1.39 | 0.81 | −30.1 | 0.61 | −51.0 | 0.64 | −46.1 | 1.58 | 23.4 |
| (3 mg/kg p.o.) | 0.22 | 0.13* | 18.8 | 0.10* | 11.5 | 0.06* | 9.0 | 0.14* | 12.5 |
| | | (KW) | | | | | | | |
| NT-4/5 | 1.51 | 0.73 | −48.8 | 0.84 | −40.9 | 1.07 | −28.0 | 1.13 | −25.2 |
| (20 mg/kg s.c) | 0.14 | 0.04* | 6.0 | 0.14* | 14.1 | 0.09 | 8.4 | 0.19* | 12.8 |
| | | (KW) | | | | | | | |

Values are expressed as mean ± SEM
Statistics: One-way analysis of variance (ANOVA) followed by a Dunnett's test or Kruskal-Wallis one-way analysis of variance on ranks (KW) followed by a Dunn's test.
*p < 0.05 as compared to vehicle

TABLE 5
Effect of NT-4/5 on cholesterol levels (mM)

| | | | % effect | | % effect | | % effect | | % effect |
|---|---|---|---|---|---|---|---|---|---|
| | T0 | T5 | T5-T0 | T12 | T12-T0 | T19 | T19-T0 | T26 | T26-T0 |
| Day | 0 | 5 | | 12 | | 19 | | 26 | |
| Vehicle | 3.07 | 3.24 | 4.9 | 3.27 | 5.9 | 2.75 | −6.1 | 2.77 | −5.5 |
| (s.c.) | 0.14 | 0.35 | 7.8 | 0.31 | 6.0 | 0.08 | 4.7 | 0.10 | 4.9 |
| Rosiglitazone | 3.27 | 3.48 | 6.5 | 3.25 | −0.3 | 3.01 | −7.5 | 3.05 | −6.7 |
| (3 mg/kg p.o.) | 0.17 | 0.23 | 5.3 | 0.22 | 4.3 | 0.15 | 3.0 | 0.23 | 5.8 |
| NT-4/5 | 3.42 | 2.85 | −16.4 | 2.66 | −23.7 | 2.40 | −31.7 | 2.67 | −23.5 |
| (20 mg/kg s.c) | 0.11 | 0.13 | 3.4 | 0.15 | 5.0 | 0.09 | 2.0 | 0.12 | 3.9 |

Values are expressed as mean ± SEM
Statistics: One-way analysis of variance (ANOVA) followed by a Dunnett's test.
*p < 0.05 as compared to vehicle

As shown in Table 4, the blood triglyceride levels were reduced to below normal level (1.2±0.08 mM, determined by measuring 8 normal healthy C57BL/6J mice) in mice treated with either Rosiglitazone or NT 4/5. As shown in Table 5, the level of blood cholesterol level in NT 4/5 treated mice also decreased about 20-30% (not statistically significant).

Serum nonesterified fatty acids (NEFA) levels were determined by calorimetric method using a non esterified fatty acids kit (ref. FA 15, Randox, France). The result of the assay is shown in Table 6. Unlike Rosiglitazone, NT 4/5 did not seem to have affected the blood level of NEFA significantly under these conditions.

TABLE 6
Effect of NT-4/5 on NEFA levels (mM) in db/db mice

| | | | % effect | | % effect | | % effect | | % effect |
|---|---|---|---|---|---|---|---|---|---|
| | T0 | T5 | T5-T0 | T12 | T12-T0 | T19 | T19-T0 | T26 | T26-T0 |
| Vehicle | 0.735 | 0.829 | 17.2 | 0.734 | 4.8 | 0.677 | −2.0 | 0.374 | −45.7 |
| (s.c.) | 0.053 | 0.052 | 10.8 | 0.053 | 10.9 | 0.068 | 9.3 | 0.060 | 8.2 |
| Rosiglitazone | 0.782 | 0.507 | −32.8 | 0.444 | −39.9 | 0.739 | −2.2 | 0.345 | −53.7 |
| (3 mg/kg p.o.) | 0.056 | 0.034* | 6.1 | 0.044* | 8.3 | 0.033 | 7.3 | 0.039 | 6.7 |
| NT-4/5 | 0.788 | 0.780 | −0.1 | 0.679 | −8.5 | 0.521 | −30.4 | 0.411 | −44.5 |
| (20 mg/kg s.c) | 0.032 | 0.049 | 6.8 | 0.069 | 12.5 | 0.050 | 7.5 | 0.041 | 6.7 |

Values are expressed as mean ± SEM
Statistics: One-way analysis of variance (ANOVA) followed by a Dunnett's test.
*p < 0.05 as compared to vehicle

These results demonstrate that NT 4/5 significantly reduced the blood triglyceride levels in these mice.

3. NT 4/5 Improves Insulin Resistance in db/db Mice

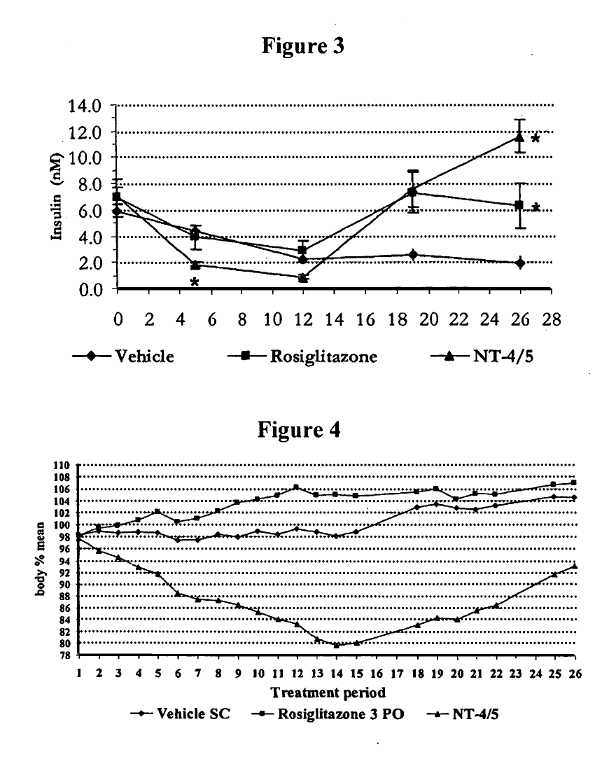

The blood insulin level was determined by ELISA using an Insulin ELIT Plus kit (ref. INSRAT01-8N, Eurobio, France). Table 7 summarizes the results of the analysis. The results are also presented graphically in FIG. 3.

TABLE 7
Effect of NT-4/5 on insulin levels (nM) in db/db mice

| | | | % effect | | % effect | | % effect | | % effect |
|---|---|---|---|---|---|---|---|---|---|
| | T0 | T5 | T5-T0 | T12 | T12-T0 | T19 | T19-T0 | T26 | T26-T0 |
| Vehicle | 5.85 | 4.35 | −22.5 | 2.29 | −59.0 | 2.58 | −57.0 | 1.96 | −69.0 |
| (s.c.) | 0.76 | 0.45 | 6.1 | 0.31 | 5.9 | 0.46 | 5.3 | 0.45 | 4.3 |
| Rosiglitazone | 6.93 | 3.94 | −37.0 | 2.85 | −51.8 | 7.30 | 14.4 | 6.33 | −5.7 |
| (3 mg/kg | 1.45 | 0.91 | 10.1 | 0.80 | 14.9 | 1.55 | 18.8 | 1.69* | 16.7 |
| p.o.) | | | | | | | | | |
| NT-4/5 | 7.04 | 1.84 | −72.0 | 0.91 | −84.5 | 7.60 | 17.1 | 11.58 | 73.1 |
| (20 mg/kg s.c.) | 0.63 | 0.16* | 3.8 | 0.18 | 5.4 | 1.43 | 20.9 | 1.26* | 14.2 |

Values are expressed as mean ± SEM
Statistics: One-way analysis of variance (ANOVA) followed by a Dunnett's test.
*p < 0.05 as compared to vehicle

The blood insulin level of vehicle treated db/db mice decreased to 4.35 mM at T5, 2.29 mM at T12, 2.58 mM at T19, and 1.96 mM at T26. In Rosiglitazone treated mice, the blood insulin level decreased to 3.94 mM at T5, 2.85 mM at T12. At T19, the blood insulin level in Rosiglitazone treated mice bounced back to 7.30 mM at T19, and 6.33 mM at T26. In NT 4/5 treated mice, the blood insulin level decreased to 1.84 mM at T5 (statistically significant as compared to vehicle), and 0.91 mM at T12. At T19, the blood insulin level in NT 4/5 treated mice bounced back to 7.6 mM and increased even further to 11.58 mM at T26.

These results demonstrate that the insulin level was suppressed in NT-4/5 treated mice. However, the insulin level was not reduced to normal level (about 0.118±0.010 nM, determined by measuring 8 normal healthy C57BL/6J mice) by NT-4/5 treatment under these conditions in these mice. The increase of insulin level after the NT 4/5 treatment was stopped may represent rebound hyper-insulinemia and increased insulin reserve of pancreatic [symbol] cells.

4. AST and ALT Enzymatic Activities of NT 4/5 in db/db Mice

Aspartate amino-transferase (AST) and alanine amino-transferase (ALT) activities were determined with an AST kit (ref. 442665, Beckman Coulter, France), an ALT kit (ref. 442620, Beckman Coulter, France), using a Synchron CX4 analyzer. Results of the experiment are shown in Table 8. The ratios of AST/ALT at each time point are shown in Table 9.

TABLE 8
Effect of NT-4/5 on serum transaminase activity in db/db mice

| | | | % effect | | % effect | | % effect | | % effect |
|---|---|---|---|---|---|---|---|---|---|
| | T0 | T5 | T5-T0 | T12 | T12-T0 | T19 | T19-T0 | T26 | T26-T0 |
| | ALT (IU/L) | | | | | | | | |
| Vehicle | 140.8 | 109.5 | −18.0 | 106.0 | −17.5 | 104.3 | −16.3 | 138.6 | 1.8 |
| (s.c.) | 13.4 | 12.2 | 10.2 | 13.2 | 14.3 | 13.7 | 13.7 | 26.6 | 11.3 |
| Rosiglitazone | 157.5 | 172.3 | 12.7 | 222.0 | 43.6 | 179.0 | 16.6 | 144.3 | −10.1 |
| (3 mg/kg p.o.) | 13.2 | 18.7* | 13.9 | 32.2* | 19.5 | 24.2* | 18.1 | 20.9 | 8.6 |
| NT-4/5 | 195.0 | 102.8 | −45.8 | 89.7 | −55.1 | 92.3 | −51.8 | 124.3 | −38.2 |
| (20 mg/kg s.c) | 17.2 | 16.0 | 9.0 | 24.5 | 10.1 | 18.5 | 11.3 | 20.2 | 5.0 |
| | AST (IU/L) | | | | | | | | |
| Vehicle | 131.8 | 123.8 | 5.2 | 121.5 | −2.4 | 117.4 | 5.0 | 94.6 | −17.1 |
| (s.c.) | 16.3 | 12.9 | 16.5 | 22.6 | 21.1 | 10.8 | 15.0 | 7.2 | 7.9 |
| Rosiglitazone | 185.8 | 174.0 | 8.2 | 200.5 | 19.9 | 148.5 | −6.9 | 143.0 | −13.8 |
| (3 mg/kg p.o.) | 36.9 | 20.7 | 16.0 | 29.4 | 17.2 | 18.5 | 17.9 | 15.6 | 9.7 |
| NT-4/5 | 184.8 | 160.0 | 0.1 | 109.3 | −20.4 | 140.7 | −0.8 | 131.0 | −9.9 |
| (20 mg/kg s.c) | 25.4 | 35.7 | 28.4 | 12.6 | 19.0 | 18.5 | 22.0 | 12.2 | 17.0 |

Values are expressed as mean ± SEM
Statistics: One-way analysis of variance (ANOVA) followed by a Dunnett's test.
*p < 0.05 as compared to vehicle

TABLE 9
Effect of NT-4/5 on ALT/AST ratio in db/db mice

| | | | % effect | | % effect | | % effect | | % effect |
|---|---|---|---|---|---|---|---|---|---|
| | T0 | T5 | T5-T0 | T12 | T12-T0 | T19 | T19-T0 | T26 | T26-T0 |
| Vehicle | 1.13 | 0.90 | −16.7 | 0.98 | −11.7 | 0.91 | −17.3 | 1.46 | 35.8 |
| (s.c.) | 0.11 | 0.09 | 9.3 | 0.11 | 8.5 | 0.11 | 11.8 | 0.24 | 27.3 |
| Rosiglitazone | 1.00 | 1.03 | 16.4 | 1.13 | 30.4 | 1.31 | 52.2 | 1.05 | 17.1 |
| (3 mg/kg p.o.) | 0.14 | 0.11 | 19.0 | 0.09 | 22.1 | 0.24 | 36.3 | 0.14 | 21.2 |
| NT-4/5 | 1.17 | 0.73 | −32.7 | 0.87 | −26.6 | 0.67 | −45.6 | 0.99 | −17.6 |
| (20 mg/kg s.c) | 0.16 | 0.11 | 12.0 | 0.23 | 18.8 | 0.10 | 8.6 | 0.20 | 17.0 |

Values are expressed as mean ± SEM
Statistics: One-way analysis of variance (ANOVA)

As shown in Tables 8 and 9, NT 4/5 did not have significant effect on either the AST and/or ALT levels, or the AST/ALT ratios. This result indicates that NT-4/5 treatment did not have significant liver toxicity.

5. Effect of NT 4/5 Treatment on Body Weight and Food Intake in db/db Mice

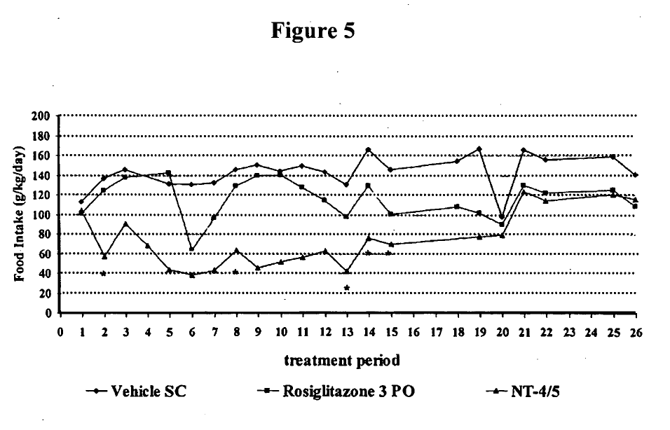

Throughout the experiment period (T1-T26), daily individual body weights as well as food intake per cage were measured. The result of the measurements are shown in Tables 10-12, and graphically presented in FIGS. 4 and 5.

TABLE 10
Effect of NT-4/5 on body weight of db/db mice

| | | Day | | | | | | | | | | | |
|---|---|---|---|---|---|---|---|---|---|---|---|---|---|
| | | 0 | 1 | 2 | 3 | 4 | 5 | 6 | 7 | 8 | 9 | 10 | 11 |
| Vehicle (s.c.) | mean | 41.8 | 41.0 | 41.3 | 41.2 | 41.2 | 41.2 | 40.7 | 40.7 | 41.1 | 40.9 | 41.3 | 41.1 |
| | sem | 0.8 | 0.7 | 0.8 | 0.8 | 0.7 | 0.8 | 0.7 | 0.8 | 0.8 | 0.8 | 0.9 | 1.0 |
| | n | 8 | 8 | 8 | 8 | 8 | 8 | 8 | 8 | 8 | 8 | 8 | 8 |
| Rosiglitazone | mean | 42.3 | 41.6 | 42.1 | 42.3 | 42.7 | 43.3 | 42.6 | 42.8 | 43.3 | 43.9 | 44.1 | 44.4 |
| (3 mg/kg p.o.) | sem | 1.2 | 1.1 | 1.2 | 1.2 | 1.2 | 1.3 | 1.3 | 1.3 | 1.3 | 1.3 | 1.3 | 1.4 |
| | n | 8 | 8 | 8 | 8 | 8 | 8 | 8 | 8 | 8 | 8 | 8 | 8 |
| NT-4/5 | mean | 41.8 | 40.8 | 40.0 | 39.5 | 38.8 | 38.3 | 37.0 | 36.5 | 36.5 | 36.6 | 36.1 | 35.6 |
| (20 mg/kg s.c.) | sem | 0.8 | 0.8 | 0.8 | 0.8 | 0.8 | 0.7 | 0.8 | 0.8 | 1.0 | 1.1 | 1.0 | 1.1 |
| | n | 8 | 8 | 8 | 8 | 8 | 8 | 8 | 8* | 7* | 6 | 6* | 6* |

| | | Day | | | | | | | | | | |
|---|---|---|---|---|---|---|---|---|---|---|---|---|
| | | 12 | 13 | 14 | 15 | 18 | 19 | 20 | 21 | 22 | 25 | 26 |
| Vehicle (s.c.) | mean | 41.4 | 41.2 | 40.9 | 41.2 | 42.6 | 42.9 | 42.5 | 42.4 | 42.7 | 43.3 | 43.3 |
| | sem | 1.0 | 1.1 | 1.2 | 1.2 | 1.1 | 1.1 | 1.0 | 1.1 | 1.0 | 1.2 | 1.2 |
| | n | 8 | 8 | 8 | 8 | 7 | 7 | 7 | 7 | 7 | 7 | 7 |
| Rosiglitazone | mean | 45.0 | 44.4 | 44.5 | 44.4 | 44.7 | 44.9 | 44.2 | 44.6 | 44.5 | 45.2 | 45.3 |
| (3 mg/kg p.o.) | sem | 1.5 | 1.5 | 1.6 | 1.5 | 1.5 | 1.4 | 1.4 | 1.5 | 1.5 | 1.5 | 1.6 |
| | n | 8 | 8 | 8 | 8 | 8 | 8 | 8 | 8 | 8 | 8 | 8 |
| NT-4/5 | mean | 35.2 | 34.2 | 33.7 | 33.9 | 35.1 | 35.7 | 35.6 | 36.2 | 36.5 | 38.8 | 39.4 |
| (20 mg/kg s.c.) | sem | 1.1 | 1.1 | 1.1 | 1.2 | 1.2 | 1.2 | 1.2 | 1.3 | 1.2 | 1.3 | 1.2 |
| | n | 6* | 6* | 6* | 6* | 6* | 6* | 6* | 6* | 6* | 6 | 6 |

Values are expressed as mean ± SEM
Statistics: One-way analysis of variance (ANOVA) followed by a Dunnett's test.
*p < 0.05 as compared to vehicle.

TABLE 11
Effect of NT-4/5 on body weight (expressed as % of baseline) of db/db mice. Data
from Table 10 was expressed as percentage of body weight on Day 0.

| | | Day | | | | | | | | | | |
|---|---|---|---|---|---|---|---|---|---|---|---|---|
| | | 1 | 2 | 3 | 4 | 5 | 6 | 7 | 8 | 9 | 10 | 11 |
| Vehicle (s.c.) | mean | 98.3 | 99.0 | 98.7 | 98.8 | 98.7 | 97.6 | 97.6 | 98.5 | 98.1 | 99.0 | 98.5 |
| | sem | 0.4 | 0.4 | 0.5 | 0.7 | 1.1 | 1.4 | 1.5 | 1.8 | 1.8 | 2.0 | 2.2 |
| | n | 8 | 8 | 8 | 8 | 8 | 8 | 8 | 8 | 8 | 8 | 8 |
| Rosiglitazone | mean | 98.3 | 99.5 | 100.0 | 100.8 | 102.2 | 100.6 | 101.1 | 102.3 | 103.8 | 104.2 | 104.9 |
| (3 mg/kg p.o.) | sem | 0.4 | 0.3 | 0.4 | 0.6 | 0.6 | 0.9 | 0.8 | 0.8 | 0.9 | 0.8 | 0.9 |
| | n | 8 | 8 | 8 | 8 | 8 | 8 | 8 | 8 | 8 | 8 | 8 |
| NT-4/5 | mean | 97.8 | 95.7 | 94.6 | 92.9 | 91.6 | 88.5 | 87.4 | 87.3 | 86.5 | 85.4 | 84.1 |
| (20 mg/kg s.c.) | sem | 0.3 | 0.3 | 0.2 | 0.2 | 0.2 | 0.3 | 0.4 | 0.8 | 1.0 | 0.9 | 1.0 |
| | n | 8 | 8 | 8 | 8 | 8 | 8 | 8 | 7 | 6 | 6 | 6 |
| | | Day | | | | | | | | | | |
| | | 12 | 13 | 14 | 15 | 18 | 19 | 20 | 21 | 22 | 25 | 26 |
| Vehicle (s.c.) | mean | 99.4 | 98.8 | 98.2 | 98.8 | 103.0 | 103.6 | 102.8 | 102.5 | 103.2 | 104.6 | 104.5 |
| | sem | 2.4 | 2.6 | 2.8 | 3.1 | 1.2 | 1.3 | 1.2 | 1.6 | 1.7 | 2.1 | 1.8 |
| | n | 8 | 8 | 8 | 8 | 7 | 7 | 7 | 7 | 7 | 7 | 7 |
| Rosiglitazone | mean | 106.3 | 104.9 | 105.1 | 104.9 | 105.5 | 106.0 | 104.3 | 105.3 | 105.1 | 106.7 | 106.9 |
| (3 mg/kg p.o.) | sem | 1.3 | 1.6 | 1.4 | 1.3 | 0.9 | 1.0 | 0.8 | 1.0 | 1.0 | 1.2 | 1.2 |
| | n | 8 | 8 | 8 | 8 | 8 | 8 | 8 | 8 | 8 | 8 | 8 |
| NT-4/5 | mean | 83.3 | 80.8 | 79.8 | 80.1 | 83.1 | 84.4 | 84.1 | 85.6 | 86.4 | 91.7 | 93.1 |
| (20 mg/kg s.c.) | sem | 1.1 | 1.3 | 1.4 | 1.6 | 1.8 | 1.8 | 1.9 | 2.0 | 1.9 | 1.8 | 1.7 |
| | n | 6 | 6 | 6 | 6 | 6 | 6 | 6 | 6 | 6 | 6 | 6 |

TABLE 12
Effect of NT-4/5 on food intake (g/kg/day) of db/db mice

| | | Day | | | | | | | | | | |
|---|---|---|---|---|---|---|---|---|---|---|---|---|
| | | 1 | 2 | 3 | 4 | 5 | 6 | 7 | 8 | 9 | 10 | 11 |
| Vehicle (s.c.) | mean | 113.1 | 137.1 | 146.1 | | 131.7 | 131.2 | 132.3 | 145.9 | 151.0 | 144.3 | 150.0 |
| | sem | 22.9 | 17.7 | 14.2 | | 19.5 | 15.1 | 65.2 | 26.6 | 23.8 | 26.9 | 27.6 |
| | n | 2 | 2 | 2 | | 2 | 2 | 2 | 2 | 2 | 2 | 2 |
| Rosiglitazone | mean | 100.8 | 124.9 | 137.8 | | 143.1 | 63.4 | 96.6 | 129.1 | 139.3 | 140.2 | 128.1 |
| (3 mg/kg p.o.) | sem | 9.7 | 5.9 | 9.5 | | 16.6 | 31.5 | 10.0 | 7.6 | 3.3 | 5.1 | 4.0 |
| | n | 2 | 2 | 2 | | 2 | 2 | 2 | 2 | 2 | 2 | 2 |
| NT-4/5 | mean | 104.1 | 56.9 | 90.7 | 68.6 | 44.1 | 38.5 | 43.1 | 63.4 | 45.6 | 51.8 | 56.2 |
| (20 mg/kg s.c.) | sem | 11.9 | 7.0 | 4.6 | 3.0 | 5.2 | 5.0 | 24.4 | 4.7 | 1.4 | 1.2 | 1.2 |
| | n | 2 | 2* | 2 | 2 | 2 | 2 | 2 | 2* | 2 | 2 | 2 |

| | | Day | | | | | | | | | | |
|---|---|---|---|---|---|---|---|---|---|---|---|---|
| | | 12 | 13 | 14 | 15 | 18 | 19 | 20 | 21 | 22 | 25 | 26 |
| Vehicle (s.c.) | mean | 143.8 | 131.3 | 166.1 | 145.9 | 154.1 | 166.9 | 98.1 | 166.0 | 156.2 | 159.3 | 140.8 |
| | sem | 24.1 | 15.4 | 21.3 | 21.8 | 22.7 | 60.5 | 29.7 | 10.0 | 4.1 | 9.6 | 3.0 |
| | n | 2 | 2 | 2 | 2 | 2 | 2 | 2 | 2 | 2 | 2 | 2 |
| Rosiglitazone | mean | 114.4 | 98.3 | 129.1 | 100.3 | 108.2 | 102.0 | 90.5 | 129.9 | 122.3 | 125.4 | 109.2 |
| (3 mg/kg p.o.) | sem | 4.0 | 15.9 | 3.7 | 2.4 | 3.5 | 0.7 | 1.6 | 1.6 | 6.7 | 0.9 | 10.0 |
| | n | 2 | 2 | 2 | 2 | 2 | 2 | 2 | 2 | 2 | 2 | 2 |
| NT-4/5 | mean | 62.6 | 42.3 | 76.3 | 69.9 | | 78.0 | 79.1 | 123.9 | 114.4 | 120.6 | 116.3 |
| (20 mg/kg s.c.) | sem | 16.3 | 10.0 | 8.0 | 3.0 | | 13.7 | 3.7 | 4.2 | 2.5 | 2.6 | 5.8 |
| | n | 2 | 2* | 2* | 2 | | 2 | 2 | 2 | 2 | 2 | 2 |

As shown in FIG. 4 and Tables 10 and 11, NT 4/5 treated mice gradually lost weight during the treatment. Body weights started to increase at about three days after the stop of NT 4/5 treatment, but remained much lower than those of vehicle treated mice and Rosiglitazone treated mice. Similarly, as shown in FIG. 5 and Table 12, the food intakes of N/T treated mice were dramatically suppressed. Food intakes started to increase at about three days after the stop of NT 4/5 treatment, but remained much lower than those of vehicle treated mice and Rosiglitazone treated mice.

These data demonstrate that NT 4/5 significantly suppressed weight gain and food intake in db/db mice.

6. Effect of NT 4/5 of Muscle Weight in db/db Mice

Heart weight and gastrocnemius weight were measured on day 28 for vehicle, Rosiglitazone, and NT-4/5 treated mice. The results are shown in Table 13. As shown in Table 13, NT-4/5 treatment did not significantly change heart muscle and gastrocnemius weight as compared to vehicle treatment. These indicate no muscle wasting or loss of lean body mass during the NT-4/5 treatment.

TABLE 13
Effect of NT-4/5 on weight muscle (g) in db/db mice

| | Heart weight | Gastrocnemius weight |
|---|---|---|
| | (g) | (g) |
| Vehicle | 0.104 | 0.074 |
| (s.c.) | 0.003 | 0.002 |
| Rosiglitazone | 0.114 | 0.074 |
| (3 mg/kg p.o.) | 0.005 | 0.003 |
| NT-4/5 | 0.104 | 0.070 |
| (20 mg/kg s.c.) | 0.002 | 0.002 |

Values are expressed s mean ± SEM
Statistics: One-way analysis of variance (ANOVA)

### Example 2

### Dose Response of NT-4/5 on db/db Mice

### A. Experimental Protocol

Db/db mice maintained as described in Example 1 were divided into five groups (groups 1-5). Group 1 (7 mice) were administered with vehicle (PBS). Group 2 (7 mice), group 3 (8 mice), group 4 (8 mice), group 5 (8 mice) were each administered with NT-4/5 at the doses of 2 mg/kg, 5 mg/kg, 10 mg/kg, and 20 mg/kg, respectively. Both the vehicle and NT-4/5 were administered to each group of the mice subcutaneously once daily for five days. One day before beginning the treatment (T0), blood samples were collected from the mice as described previously for the determination of baseline levels of serum biomarkers (glucose, triglyceride, and cholesterol). Similarly, at day 6 (T6), blood samples were collected for the determination of serum biomarker levels. The blood samples were treated in the same manner as described in Example 1.

### B. Results

### 1. Dose Response of NT-4/5 on Serum Biomarkers

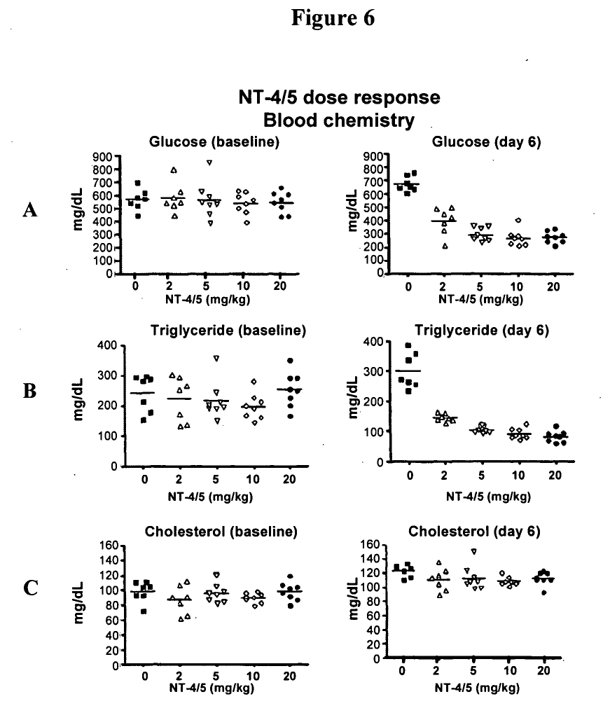

Blood glucose, triglyceride, and cholesterol levels were determined as described in Example 1. Table 14 summarizes the results of the analyses. The results are also graphically presented in FIG. 6.

TABLE 14
Dose response of NT-4/5 on serum biomarkers of db/db mice
(12 week, female)

| | | |
|---|---|---|
| Cholesterol | Glucose | Triglyceride |

Day 0 (Baseline)

| | | | |
|---|---|---|---|
| Vehicle | 98.25 | 526.63 | 235.88 |
| | 4.88 | 51.76 | 23.00 |
| NT-4/5 | 86.25 | 544.50 | 206.13 |
| (2 mg/kg) | 6.76 | 54.49 | 31.27 |
| NT-4/5 | 95.88 | 563.00 | 217.13 |
| (5 mg/kg) | 4.35 | 48.29 | 21.93 |
| NT-4/5 | 90.00 | 538.00 | 197.50 |
| (10 mg/kg) | 2.20 | 28.62 | 15.16 |
| NT-4/5 | 98.38 | 544.88 | 253.50 |
| (20 mg/kg) | 4.35 | 28.38 | 20.83 |

Day 6

| | | | |
|---|---|---|---|
| Vehicle | 123.00 | 672.43 | 300.43 |
| | 2.93 | 21.79 | 22.19 |
| NT-4/5 | 111.43 | 396.86 | 144.43 |
| (2 mg/kg) | 5.91 | 37.86*** | 4.65*** |
| NT-4/5 | 113.25 | 295.13 | 104.25 |
| (5 mg/kg) | 5.94 | 17.16*** | 3.62*** |
| NT-4/5 | 108.75 | 270.38 | 90.75 |
| (10 mg/kg) | 2.02 | 21.44*** | 6.18*** |
| NT-4/5 | 112.88 | 276.75 | 81.75 |
| (20 mg/kg) | 3.30 | 15.47*** | 6.77*** |

Value are expressed as mean +/− SEM
Statistics: One way analysis of variance (ANOVA) followed by a Dunnett's test
*P < 0.05
**P < 0.01
***P < 0.001

The baseline blood glucose levels of the mice were about 500-600 mg/dL. Administration of 2 mg/kg of NT-4/5 significantly decreased the blood glucose level to about 400 mg/dL. NT-4/5 at 5 mg/kg, 10 mg/kg, and 20 mg/kg further decreased the levels to less than 300 mg/dL. The baseline blood triglyceride levels of the mice were about 200-300 mg/dL. Administration of 2 mg/kg of NT-4/5 significantly decreased the blood triglyceride level to about 150 mg/dL on day 6. NT-4/5 at 5 mg/kg, 10 mg/kg, and 20 mg/kg further decreased the levels to about 100 mg/dL or less on day 6. In contrast, the blood cholesterol levels of NT-4/5 treated mice remained at the baseline level.

In another experiment, db/db mice were subcutaneously injected with vehicle (PBS) or NT-4/5 at 2 mg/kg, 5 mg/kg, 10 mg/kg, or 20 mg/kg daily for two weeks. Significant reduction of triglyceride level was observed in all NT-4/5 treated groups on day 14 as compared to the vehicle control group.

2. Dose Response of NT-4/5 on Food Intake and Body Weight of db/db Mice

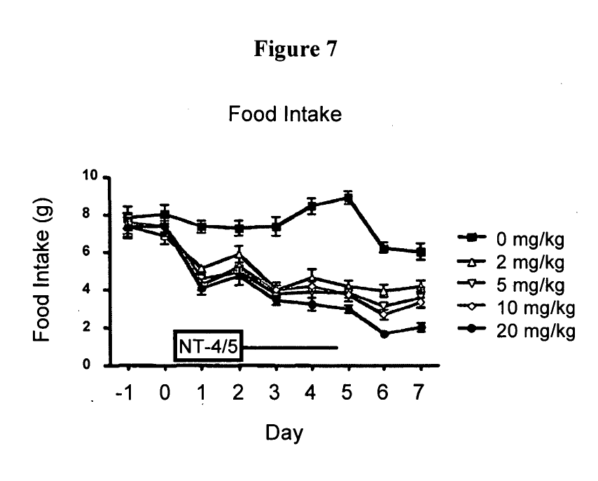

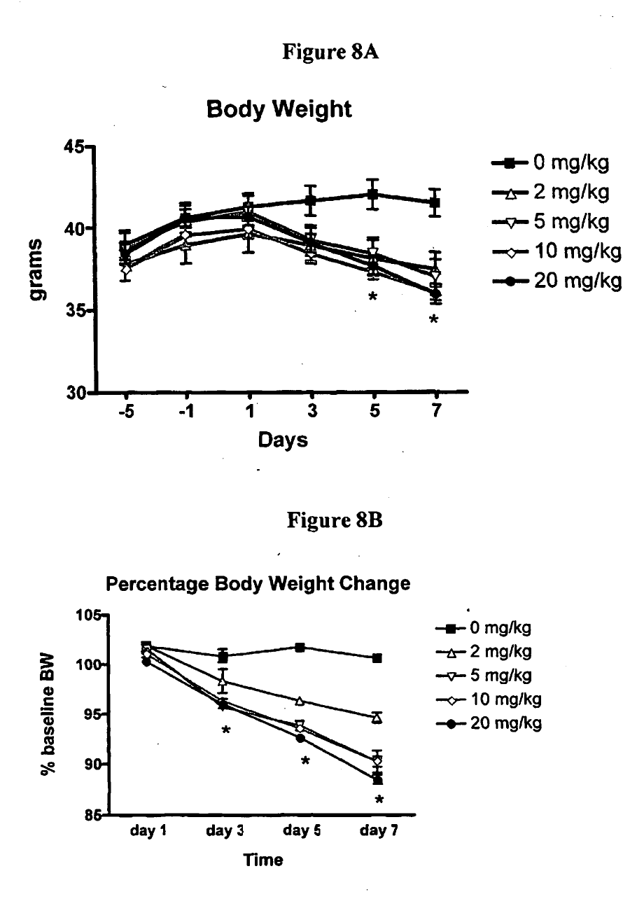

From two days before the treatment (T-1) to day 6 (T6), individual food intakes of the mice were measured daily. The results of the measurements are shown in FIG. 7 and Table 15. Individual body weights were also measured daily from 6 days before the treatment (T-5) to day 7 (T7). The results of the measurements are shown in FIG. 8A and Table 16. The percentage body weight changes were also calculated and plotted in FIG. 8B and summarized in Table 17.

TABLE 15
Dose response of NT-4/5 on the food intake of db/db mice (12 week, female)

| | Day 0 | Day 1 | Day 2 | Day 3 | Day 4 | Day 5 | Day 6 | Day 7 |
|---|---|---|---|---|---|---|---|---|
| Vehicle | 7.74 | 6.99 | 7.03 | 7.11 | 8.04 | 8.30 | 6.00 | 5.84 |
| | 0.65 | 0.50 | 0.44 | 0.70 | 0.78 | 0.81 | 0.49 | 0.54 |
| NT-4/5 | 7.79 | 5.20 | 5.87 | 3.89 | 4.79 | 4.19 | 3.99 | 4.13 |
| (2 mg/kg) | 0.64 | 0.18*** | 0.43 | 0.42*** | 0.42*** | 0.29*** | 0.32*** | 0.32** |
| NT-4/5 | 7.26 | 4.55 | 4.86 | 3.99 | 3.69 | 3.79 | 3.00 | 3.48 |
| (5 mg/kg) | 0.43 | 0.31*** | 0.34*** | 0.20*** | 0.26*** | 0.26*** | 0.22*** | 0.57*** |
| NT-4/5 | 7.48 | 4.13 | 5.18 | 3.85 | 4.00 | 3.60 | 2.56 | 3.21 |
| (10 mg/kg) | 0.35 | 0.29*** | 0.25*** | 0.23*** | 0.42*** | 0.34*** | 0.32*** | 0.30*** |
| NT-4/5 | 7.46 | 4.11 | 4.66 | 3.39 | 3.40 | 3.06 | 1.71 | 2.03 |
| (20 mg/kg) | 0.52 | 0.36*** | 0.51*** | 0.28*** | 0.32*** | 0.20*** | 0.17*** | 0.26*** |

Value are expressed as mean +/− SEM
Statistics: Two way analysis of variance (ANOVA) followed by a Bonferroni's test
*P < 0.05 as compared to vehicle group
**P < 0.01
***P < 0.001

TABLE 16
Dose response of NT-4/5 on the body weight
of db/db mice (12 week, female)

| | Day −1 | Day 1 | Day 3 | Day 5 | Day 7 |
|---|---|---|---|---|---|
| Vehicle | 40.63 | 41.31 | 41.66 | 42.03 | 41.53 |
| | 0.93 | 0.82 | 0.89 | 0.89 | 0.81 |
| NT-4/5 | 38.94 | 39.66 | 38.96 | 38.17 | 37.50 |
| (2 mg/kg) | 1.08 | 1.11 | 1.10 | 1.11* | 1.05** |
| NT-4/5 | 40.41 | 41.03 | 39.26 | 38.48 | 37.00 |
| (5 mg/kg) | 0.93 | 0.96 | 0.93 | 0.93* | 1.07*** |
| NT-4/5 | 39.58 | 39.99 | 38.49 | 37.35 | 36.04 |
| (10 mg/kg) | 0.65 | 0.59 | 0.47* | 0.48*** | 0.47*** |
| NT-4/5 | 40.60 | 40.73 | 39.10 | 37.71 | 36.00 |
| (20 mg/kg) | 0.53 | 0.56 | 0.58 | 0.56** | 0.59*** |

Value are expressed as mean SEM
Statistics: Two way analysis of variance (ANOVA) followed by a Bonferroni's test
*P < 0.05 as compared to vehicle group
**P < 0.01
***P < 0.001

TABLE 17
Dose response of NT-4/5 on the percentage change in body
weight of db/db mice (12 week, female)

| | Day 1 | Day 3 | Day 5 | Day 7 |
|---|---|---|---|---|
| Vehicle | 101.73 | 100.82 | 101.72 | 100.53 |
| | 0.48 | 0.61 | 0.32 | 0.28 |
| NT-4/5 (2 mg/kg) | 101.83 | 98.27 | 96.24 | 94.57 |
| | 0.24 | 1.21** | 0.34*** | 0.48*** |
| NT-4/5 (5 mg/kg) | 101.51 | 95.71 | 93.79 | 90.15 |
| | 0.44 | 0.37*** | 0.51*** | 1.09*** |
| NT-4/5 (10 mg/kg) | 101.07 | 96.28 | 93.43 | 90.14 |
| | 0.45 | 0.32*** | 0.39*** | 0.48*** |
| NT-4/5 (20 mg/kg) | 100.31 | 96.01 | 92.60 | 88.38 |
| | 0.34 | 0.43*** | 0.28*** | 0.47*** |

Value are expressed as mean +/− SEM
Statistics: Two way analysis of variance (ANOVA) followed by a Bonferroni's test
*P < 0.05
**P < 0.01
***P < 0.001

The food intakes of the NT-4/5 treated mice were already significantly decreased on the first day of NT-4/5 treatment, and the decrease continued until the end of the experiment period. Treatment with NT-4/5 at 2 mg/kg, 5 mg/kg, 10 mg/kg, and 20 mg/kg significantly decreased food intake as compared to vehicle. Similarly, the body weights of the treated mice started to decrease at day 3 of the treatment, and the decrease continued until the end of the experiment period. As shown in FIG. 8A and FIG. 8B, treatment with NT-4/5 at 2 mg/kg, 5 mg/kg, 10 mg/kg, and 20 mg/kg significantly decreased body weight as compared to vehicle.

Female db/db mice (12 weeks old) were divided into four groups and were subcutaneously injected with vehicle (PBS) or NT-4/5 at doses of 2 mg/kg, 5 mg/kg, or 10 mg/kg daily from day 0 to day 4, and then on day 8, day 11, day 15, day 18, day 22, day 25, and day 29. NT-4/5 administration reduced body weight in a dose dependent manner. In addition, the body weights in NT-4/5 in all treated groups continued to be reduced during the treatment period. On day 31, about 20% body weight reduction was observed in 5 mg/kg and 10 mg/kg NT-4/5 injected group.

Female db/db mice (12 weeks old) were divided into three groups and were subcutaneously injected with vehicle (PBS) or NT-4/5 at doses of 5 mg/kg, or 20 mg/kg daily from day 1 to day 30. NT-4/5 administration reduced body weight in a dose dependent manner. Food intake was reduced to about 20% of the vehicle group level after 10 days of administration of NT-4/5 at 20 mg/kg and was maintained at that level until day 30. Food intake was returned to about the same level as the vehicle group about 12 days after stop of treatment.

### Example 3

### Effect of NT-4/5 on Carbohydrate Metabolism and Body Weight Homeostasis in the Polygenic Obese Mice (NONcNZO-10)

### A. Experimental Protocol

Test animals: 19 male NONcNZO-10 polygenic obese and diabetic mice from Jackson Laboratory (See Leiter et al., Diabetes 53 (Suppl. 1): S4-S11, 2004) at 9 weeks of age weighing in the range of 28-35 g were used in this study. These mice were housed in a temperature (19.5-24.5° C.) and relative humidity (45-65%) controlled room with a 12-hour light/dark cycle, with ad libitu access to filtered tap-water and irradiated pelleted laboratory chow (PURINA) throughout the study. Upon receipt at animal facilities, they were housed 1 per cage covered with filtered caps and at least a 5-day acclimation period was observed.

Administration of NT-4/5: NT-4/5 was obtained from Genentech Inc.

The mice were divided into 3 groups of equivalent body weight and baseline glucose distribution (6-7 mice per group). Each group received, from day 1 to day 5 and then from day 8 to day 11, a daily subcutaneous dose of either vehicle, 2 mg/kg of NT-4/5 or 10 mg/kg of NT-4/5. Seven days before starting the treatment (day −7), blood samples were collected from the mice as described previously for the determination of baseline levels of blood glucose and triglyceride. After the treatment started, blood samples were collected weekly to determine glucose and triglyceride levels as described in previous examples. Body weight and food intake were monitored 5 days a week through out the entire study.

### B. Results

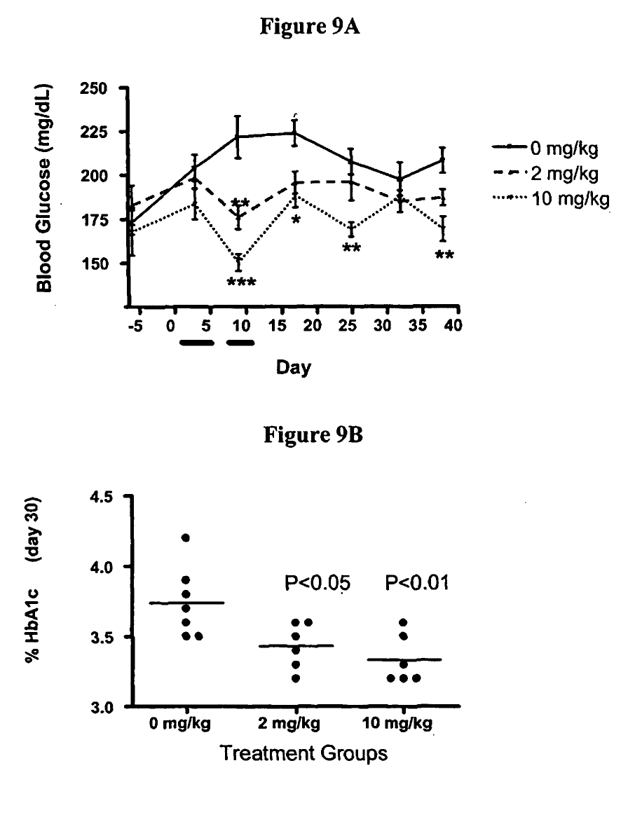

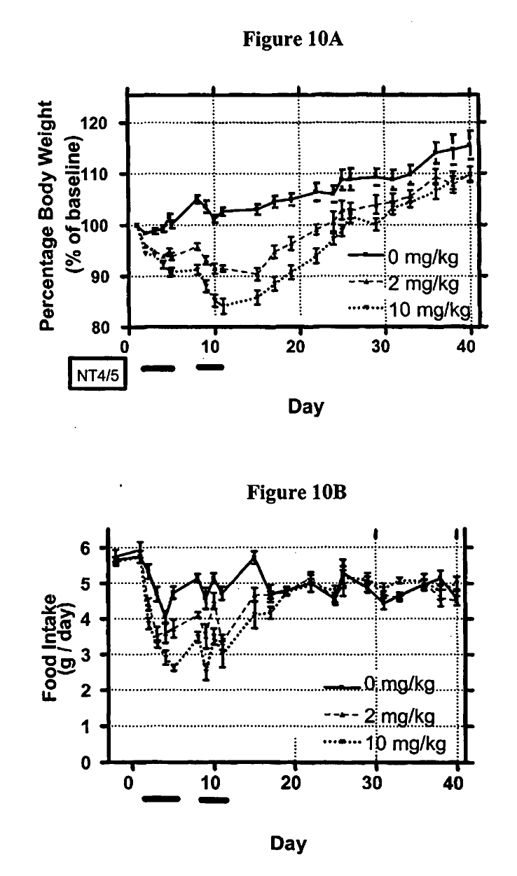

As shown in FIG. 9, NT-4/5 reduced non-fasting blood glucose level (FIG. 9A) and showed improved long term glycemic control as indicated by HbA1c levels (FIG. 9B) in a dose dependent manner in these NONcNZO-10 polygenic obese mice. NT-4/5 also reduced body weight (FIG. 10A) and food intake (FIG. 10B) of NONcNZO-10 polygenic obese mice in a dose dependent manner. No rebound hyperphagia or rebound body weight gain/overshoot was observed even 30 days after the treatment was terminated.

Experiment was also performed on the same type of mice by intravenous injection of vehicle (PBS) and NT-4/5 (1 mg/kg) on day 1 and on day 5. About 5% to about 10% reduction of body weight was observed between NT-4/5 treated group and vehicle group between day 2 and day 15. This experiment shows that significant body weight reduction can be maintained for up to 10 days after 2 staggered intravenous doses of NT-4/5.

### Example 4

### Effect of NT-4/5 on Lipid Metabolism Body Weight Homeostasis, Food Intake Water Intake Electrolyte Balance and Endocrine Functions in the Polygenic Obese Mice (NONcNZO-10)

Test animals: 40 male NONcNZO-10 polygenic obese mice obtained from Jackson Laboratory at 10 weeks of age weighing in the range of 26-32 g were used in this study. These mice were housed in a temperature (19.5-24.5° C.) and relative humidity (45-65%) controlled room with a 12-hour light/dark cycle, with ad libitu access to filtered tap-water and irradiated pelleted laboratory chow (PURINA) throughout the study. Upon receipt at animal facilities, they were housed 1 per cage covered with filtered caps and at least a 5-day acclimation period was observed.

Administration of NT-4/5: NT-4/5 was produced in E. coli as descried in U.S. Pat. No. 6,184,360. The mice were divided into 6 groups of equivalent body weight and baseline blood glucose distribution (6-7 mice per group). Each group received from day 1 to day 5 and then from day 8 to day 11 a daily subcutaneous dose of either vehicle, 3 mg/kg, 1 mg/kg, 0.3 mg/kg or 0.1 mg/kg of NT-4/5. A “paired-fed” control group received daily injection of vehicle, but were only fed with the same amount of food the 3 mg/kg group consumed the previous day. On the day when the treatment started (day 1), a baseline blood samples were collected for measuring glucose and triglyceride. Triglyceride level was measured on day 10. At the end of the study (day 12), a terminal blood sample was collected to determine glucose, triglyceride, electrolytes (Na, K, Cl), testoterone, corticosterone, thyroid hormone (total T4) and thyroid stimulating hormone (TSH) levels. Electrolytes, free testosterone, T4 and TSH levels of serum samples were measured by AniLytics (Gaithesburg, M.D) according to the standard protocols employed by the vendor. Other analytes were measured as described above. Body weight, food intake were monitored 5 days per week through out the entire study. Daily water intake was measured from day 5 through day 7.

### B. Results

As shown in Table 18, daily subcutaneous NT-4/5 injection significantly reduced non-fasting blood triglyceride levels. Remarkably, even the lowest daily subcutaneous dose of NT-4/5 (0.1 mg/kg/day) significantly reduced the non-fasting blood triglyceride level in NONcNZO-10 polygenic obese mice.

TABLE 18
Effect of different dosages of NT-4/5 on non-fasting blood
triglyceride level in polygenic obese mice (NONcNZO-10).

Day

| | | 1 | 10 |
|---|---|---|---|
| Vehicle (s.c.) | mean | 291.3 | 282.7 |
| | sem | 24.2 | 22.6 |
| | n | 7 | 7 |
| Vehicle (s.c.) | mean | 271.7 | 230.4 |
| paired fed to 3 mg/kg | sem | 18.5 | 22.1 |
| | n | 7 | 7* |
| 3 mg/kg of NT4 (s.c.) | mean | 306.1 | 226.3 |
| | sem | 27.3 | 28.1 |
| | n | 7 | 7* |
| 1 mg/kg of NT4 (s.c.) | mean | 307.7 | 226.3 |
| | sem | 10.8 | 26.9 |
| | n | 7 | 7 |
| 0.3 mg/kg of NT4 (s.c.) | mean | 279.3 | 208.0 |
| | sem | 14.8 | 19.3 |
| | n | 6 | 6* |
| 0.1 mg/kg of NT4 (s.c.) | mean | 291.7 | 172.7 |
| | sem | 35.6 | 24.0 |
| | n | 6 | 6** |

Values are expressed as mean ± SEM
Statistics: Two-way analysis of variance (ANOVA) followed by Bonferroni's posttest.
*P < 0.05,
**P < 0.01 as compared to vehicle.

As shown in Tables 19 and 20, NT-4/5 injection also reduced body weight (Table 19) and food intake (Table 20) of NONcNZO-10 polygenic obese mice in a dose dependent manner. The mice treated with 3 mg/kg NT-4/5 showed about 10% body weight loss and those treated with 1 mg/kg NT-4/5 showed about 5% body weight loss relative to the vehicle control. However, only the 3 mg/kg group, but not the 1 mg/kg group, showed a significant reduction in food intake. The mice in the paired fed group overall consumed a very similar amount of food as the 3 mg/kg group and yet they only lost about 5% of body weight relative to vehicle control.

TABLE 19
Effect of different dosages of NT-4/5 on body weight in polygenic obese mice
(NONcNZO-10).

| | | Day | | | | | | | | |
|---|---|---|---|---|---|---|---|---|---|---|
| | | 1 | 2 | 3 | 4 | 5 | 8 | 9 | 10 | 11 |
| Vehicle (s.c.) | mean | 100.0 | 98.6 | 100.4 | 100.5 | 101.5 | 102.5 | 103.3 | 104.3 | 103.9 |
| | sem | 0.0 | 0.7 | 0.4 | 0.5 | 1.0 | 0.9 | 1.0 | 1.3 | 1.1 |
| | n | 7 | 7 | 7 | 7 | 7 | 7 | 7 | 7 | 7 |
| Vehicle (s.c.) | mean | 100.0 | 98.0 | 98.0 | 97.6 | 99.5 | 97.6 | 96.5 | 97.2 | 96.3 |
| paired fed to 3 mg/kg | sem | 0.0 | 0.7 | 1.1 | 0.6 | 1.6 | 1.2 | 0.8 | 0.8 | 1.3 |
| | n | 7 | 7 | 7 | 7 | 7 | 7** | 7*** | 7*** | 7*** |
| 3 mg/kg of NT4 (s.c.) | mean | 100.0 | 95.6 | 94.6 | 94.6 | 94.6 | 92.2 | 92.2 | 92.7 | 92.6 |
| | sem | 0.0 | 0.6 | 0.8 | 0.7 | 1.0 | 1.0 | 0.6 | 0.9 | 0.9 |
| | n | 7 | 7 | 7*** | 7*** | 7*** | 7*** | 7*** | 7*** | 7*** |
| 1 mg/kg of NT4 (s.c.) | mean | 100.0 | 97.1 | 94.8 | 98.2 | 99.2 | 97.7 | 97.6 | 97.6 | 99.0 |
| | sem | 0.0 | 0.5 | 1.6 | 1.0 | 1.2 | 0.9 | 0.9 | 0.9 | 0.6 |
| | n | 7 | 7 | 7*** | 7 | 7 | 7** | 7*** | 7*** | 7** |
| 0.3 mg/kg of NT4 (s.c.) | mean | 100.0 | 98.3 | 98.9 | 100.6 | 101.8 | 101.2 | 102.4 | 102.9 | 104.1 |
| | sem | 0.0 | 0.8 | 1.2 | 0.6 | 1.2 | 0.7 | 1.2 | 1.4 | 2.0 |
| | n | 6 | 6 | 6 | 6 | 6 | 6 | 6 | 6 | 6 |
| 0.1 mg/kg of NT4 (s.c.) | mean | 100.0 | 97.7 | 100.0 | 102.2 | 104.5 | 102.2 | 102.8 | 103.3 | 105.1 |
| | sem | 0.0 | 0.7 | 0.9 | 0.7 | 1.1 | 1.1 | 1.4 | 1.7 | 1.7 |
| | n | 6 | 6 | 6 | 6 | 6 | 6 | 6 | 6 | 6 |

Values are expressed as mean ± SEM
Statistics: Two-way analysis of variance (ANOVA) followed by Bonferroni's posttest.
**P < 0.01,
***P < 0.001 as compared to vehicle.

TABLE 20
Effect of different dosages of NT-4/5 on food intake in polygenic obese mice
(NONcNZO-10).

| | | Day | | | | | | | |
|---|---|---|---|---|---|---|---|---|---|
| | | 1 | 2 | 3 | 4 | 5 | 8 | 9 | 10 |
| Vehicle (s.c.) | mean | 4.71 | 5.14 | 5.29 | 5.14 | 5.14 | 5.14 | 5.00 | 5.71 |
| | sem | 0.63 | 0.59 | 0.57 | 0.34 | 0.14 | 0.52 | 0.69 | 0.78 |
| | n | 7 | 7 | 7 | 7 | 7 | 7 | 7 | 7 |
| Vehicle (s.c.) | mean | 5.11 | 5.43 | 4.71 | 4.14 | 4.29 | 3.86 | 3.57 | 3.71 |
| paired fed to 3 mg/kg | sem | 0.23 | 0.48 | 0.29 | 0.14 | 0.29 | 0.14 | 0.20 | 0.29 |
| | n | 7 | 7 | 7 | 7 | 7 | 7 | 7 | 7** |
| 3 mg/kg of NT4 (s.c.) | mean | 5.13 | 4.86 | 4.29 | 4.29 | 4.00 | 3.86 | 3.57 | 3.86 |
| | sem | 0.24 | 0.26 | 0.18 | 0.29 | 0.00 | 0.18 | 0.20 | 0.26 |
| | n | 7 | 7 | 7** | 7 | 7* | 7 | 7 | 7** |
| 1 mg/kg of NT4 (s.c.) | mean | 5.07 | 5.43 | 4.86 | 4.71 | 4.43 | 3.93 | 4.00 | 4.14 |
| | sem | 0.21 | 0.30 | 0.26 | 0.29 | 0.37 | 0.13 | 0.22 | 0.26 |
| | n | 7 | 7 | 7 | 7 | 7 | 7 | 7 | 7 |
| 0.3 mg/kg of NT4 (s.c.) | mean | 5.32 | 5.67 | 5.33 | 5.67 | 4.83 | 4.33 | 4.50 | 4.67 |
| | sem | 0.30 | 0.21 | 0.33 | 0.21 | 0.17 | 0.28 | 0.34 | 0.21 |
| | n | 6 | 6 | 6 | 6 | 6 | 6 | 6 | 6 |
| 0.1 mg/kg of NT4 (s.c.) | mean | 5.17 | 5.50 | 5.83 | 6.00 | 5.50 | 4.50 | 4.83 | 5.00 |
| | sem | 0.22 | 0.22 | 0.31 | 0.26 | 0.22 | 0.34 | 0.40 | 0.45 |
| | n | 6 | 6 | 6 | 6 | 6 | 6 | 6 | 6 |

Values are expressed as mean ± SEM
Statistics: Two-way analysis of variance (ANOVA) followed by Bonferroni's posttest.
*P < 0.05,
**P < 0.01 as compared to vehicle.

The effects of NT-4/5 treatment on water intake, electrolyte balance and endocrine functions were also tested. Water intake was measured from day 5 through 7. The electrolyte (i.e., Na, K, and Cl) concentrations were measured using the terminal blood samples taken on day 12. None of the NT-4/5 treatment (0.1 mg/kg, 0.3 mg/kg, 1 mg/kg, and 3 mg/kg for water intake test; and 1 mg/kg and 3 mg/kg for Na, K, and Cl concentration test) or paired fed groups showed a statistically significant difference from the vehicle group in any of these measurements. One way ANOVA followed by Dunnett's post-test were used for statistical analysis.

Total thyroxine T4 was significantly reduced (by about 33%, while still within normal range) in the group treated by daily 3 mg/kg NT-4/5, but not in the paired-fed or 1 mg/kg treated group (one way ANOVA, followed by Dunnett's post-test). There was no change in the thyroid stimulating hormone (TSH) level in any of the experimental groups compared to the vehicle control (one way ANOVA, followed by Dunnett's post-test).

In addition, NT-4/5 treatment (0.1-3 mg/kg/day) did not significantly change the serum corticosterone levels and testoterone levels at the end of the experiment (day 12). However, the corticosterone level of the paired-fed group was significantly increased (P<0.05) to about 3-fold as compared to the vehicle group. One way ANOVA followed by Dunnett's post-test were used for statistical analysis. This is consistent with the possibility that the paired-fed group, but none of the NT-4/5 treated groups, was under stress of constant semi-starvation.

### Example 5

### Effect of NT-4/5 on Carbohydrate, Lipid and Body Weight Homeostasis in the High Fat Diet-Induced Obese Mice

### A. Experimental Protocol

Test animals: Mice with diet induced obesity (DIO) from Jackson Laboratory were used in this study. El-Haschimi, J. Clin. Invest. 105:1827-1832 (2000); Steppan, Nature 409:307-312 (2001). 23 male C57BL/6J mice were weaned at 4 weeks of age. Immediately after weaning they were put on about 58% high fat diet (D1233 μl, Research Diet) through out the study. The DIO mice at 13 weeks of age weighing in the range of 28-40 g were used in this study. Older mice at 22-24 weeks of age were also used. These mice were housed in a temperature (19.5-24.5° C.) and relative humidity (45-65%) controlled room with a 12-hour light/dark cycle, with ad libitu access to filtered tap-water and 58% high fat diet (D12331i, Research Diet) throughout the study. Upon receipt at animal facilities, they were housed 1 per cage covered with filtered caps and at least a 5-day acclimation period was observed.

Administration of NT-4/5: NT-4/5 used in this study was obtained from Genentech. The mice were divided into 3 groups of equivalent body weight and baseline blood glucose distribution (7-8 mice per group). Each group received from day 1 to day 5, from day 8 to day 12, from day 15 to 16 a daily subcutaneous dose of either vehicle, 2 mg/kg or 10 mg/kg of NT-4/5, respectively. Five days prior to the treatment started (day −5), a baseline blood samples were collected for measuring glucose and triglyceride. Subsequent to the treatment started (day 1), blood glucose and triglyceride were measured once a week. Body weight, food intake were monitored 5 days per week through out the entire study. From days 57-59, the DIO mice received a daily injection of NT-4/5 at 2 or 10 mg/kg respectively. They were fasted overnight (for 16 hours) before fasting glucose levels were measured and the oral glucose tolerance test was conducted with a load of 2 grams of glucose/kg of body weight.

### B. Results

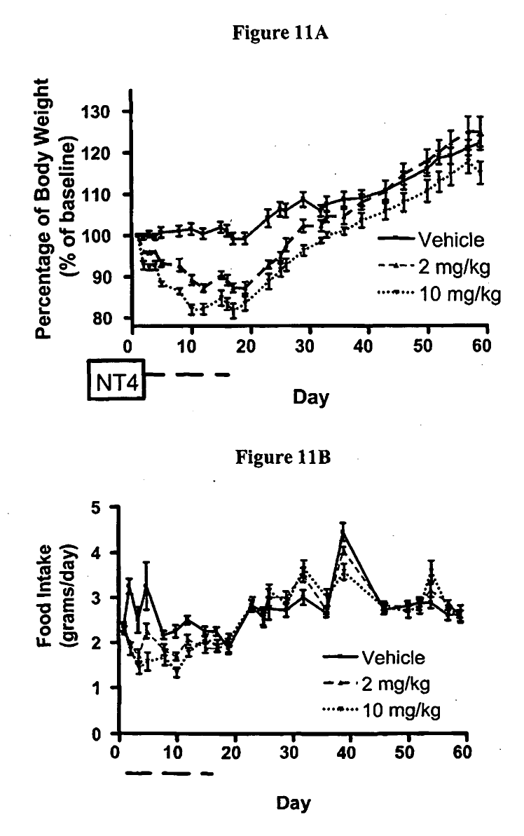

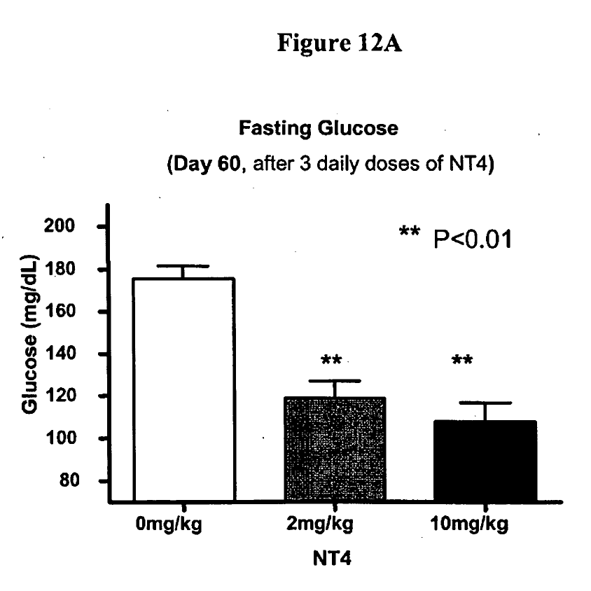

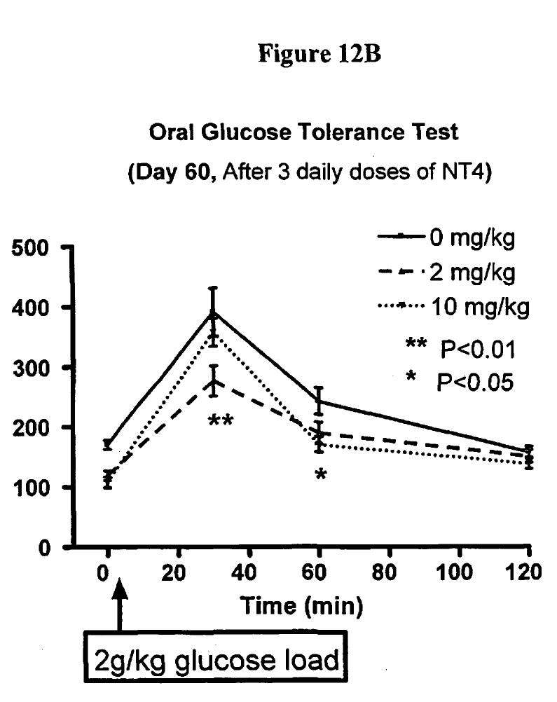

NT-4/5 administration (2 mg/kg and 10 mg/kg) significantly reduced (P<0.001, two-way ANOVA) body weight (FIG. 11A) and food intake (FIG. 11B) of DIO mice in a dose dependent manner. NT-4/5 administration (2 mg/kg and 10 mg/kg) did not affect the non-fasting glucose or triglyceride levels of the DIO mice at the younger age (12-16 weeks of age) which were not diabetic. However, as shown in FIG. 12, NT-4/5 administration (2 mg/kg and 10 mg/kg) significantly reduced fasting glucose level and improved glucose tolerance in the DIO mice at an older age (22-24 weeks of age) when the animals developed overt diabetes. Two way ANOVA followed by Bonferroni's post-test were used for statistical analysis. In a separate study, about 5% reduction of body weight was also observed after intravenous (i.v.) injection of NT-4/5 (5 mg/kg) on day 1 and day 4, and the reduced body weight was maintained for at least 10 days after the second dose was given.

### Example 6

### Effect of a Pegylated NT-4/5 Cysteine Mutant on Carbohydrate, Lipid and Body Weight Homeostasis in the Polygenic Obese Mice (NONcNZO-10)

### A. Experimental Protocol

Test animals: 30 male NONcNZO-10 polygenic obese mice at 10 weeks of age weighing in the range of 27-32 g were used in this study. These mice were housed in a temperature (19.5-24.5° C.) and relative humidity (45-65%) controlled room with a 12-hour light/dark cycle, with ad libitu access to filtered tap-water and irradiated pelleted laboratory chow (PURINA) throughout the study. Upon receipt at animal facilities, they were housed 1 per cage covered with filtered caps and at least a 5-day acclimation period was observed.

Method of making pegylated NT-4/5 (S50C): The native form of NT-4/5 used in this study was produced according to the method described in U.S. Pat. No. 6,184,360. A site-directed pegylated NT-4/5 was also produced. The structure of NT-4/5 in complex with domain 5 of TrkB was examined to find amino acid side chains that would be surface available and unlikely to interfere with natural disulfide bonds and also unlikely to interfere in either the interactions between the monomer subunits of NT-4/5 or the TrkB domain 5 to create a PEG attaching site. Based on the examination, functional binding with TrkB in the KIRA assay was found possible for the following single cysteine mutants: S50C, Q105C, and E67C. A single amino acid mutation was then introduced by changing the serine residue at position 50 of mature human NT-4/5 sequence to cysteine, resulting in a mutant form of NT-4/5 (NT4-S50C); changing the aspartic acid residue at position 105 to cysteine; or changing the glutamic acid residue at position 67 to cysteine. Mutant proteins were expressed in E. coli, solubilized, extracted, purified and refolded as the native NT-4/5. To pegylate the refolded NT4-S50C, NT4-Q105C, and NT-4-E67C, first a mild reduction with 0.1 mM DTT (dithiothreitol) was performed. Excess DTT was removed by dialysis against 50 mM sodium acetate at pH 6, then a 5 molar excess of mPEG-maleimide (M. W. of 10,000 Da, SunBio, Inc., South Korea) was incubated with the protein at 37° C. for several days with gentle agitation. Pegylated protein, non-pegylated protein and unreacted PEG were separated via ion exchange or size exclusion chromatography.

Assays of TrkB specific activation by pegylated NT-4/5: Stable cell lines expressing human TrkA, TrkB or TrkC were used to determine the functional activity, i.e. receptor phosphorylation, of NT-4/5 and pegylated NT-4/5 as previously described (Sadick et al. Experimental Cell Research 234: 354-361, 1997).

Administration of NT-4/5 and pegylated NT-4/5: The mice were divided into 4 groups of equivalent body weight distribution (7-8 mice per group). Each group received on the first day (day 1) of the study one and the only dose of either vehicle alone, 1 mg/kg of NT-4/5 or 1 mg/kg of PEG-NT4-S50C, respectively. A final group received a daily subcutaneous dose of 1 mg/kg of NT-4/5 from day 1 to day 5, then from day 7 to day 9. On day 9 after the treatment started, blood samples were collected to determine serum glucose and triglyceride levels. Body weight and food intake were monitored 5 days a week through out the entire study.

### B. Results

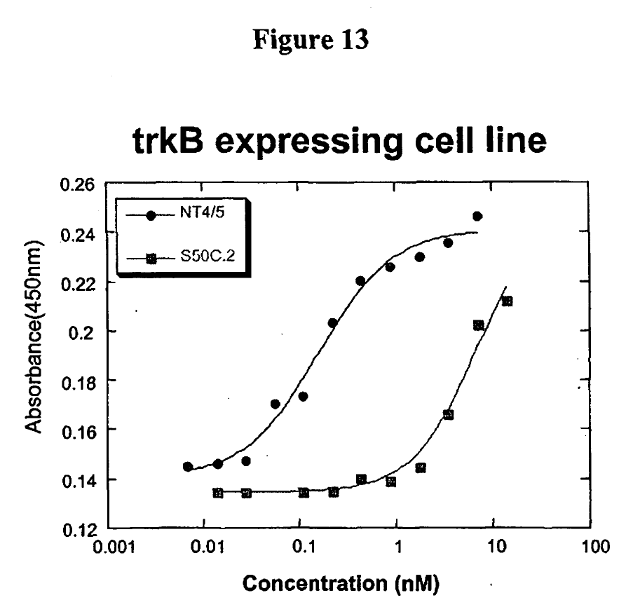

As shown FIG. 13, native NT-4/5 and PEG-NT4-S50C increased receptor tyrosine phosphorylation of TrkB-expressing cell line in a dose dependent manner. But native NT-4/5 and PEG-NT4-S50C did not increase receptor tyrosine phosphorylation of trkA- or trkC-expressing cell line. As a control for the experiment, NGF activated trkA expressing cells and NT-3 activated trkC expressing cells.

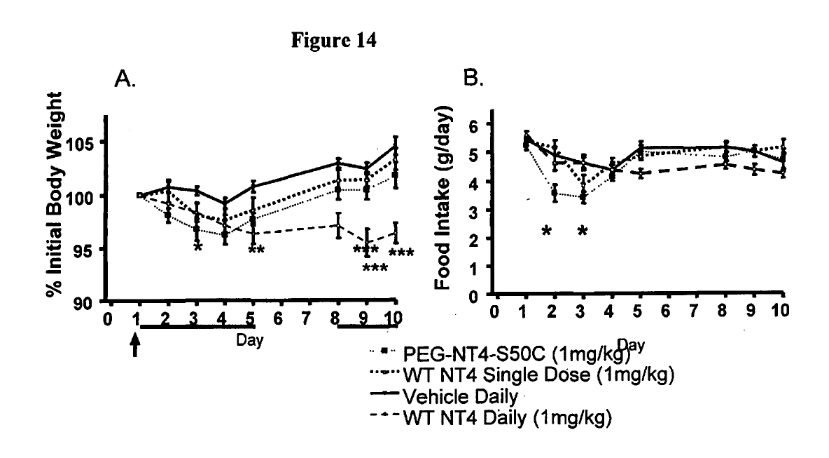

As shown in FIG. 14, a single subcutaneous injection of 1 mg/kg PEG-NT4-S50C, but not that of the wild type NT-4/5 (WT NT4), significantly reduced the food intake and body weight. By contrast, daily injection of 1 mg/kg WT NT4 significantly reduced body weight without affecting daily food intake. However, single subcutaneous injection of 1 mg/kg PEG-NT4-S50C and wild type NT-4/5 (WT NT4) did not significantly reduce blood glucose level and triglyceride level. Daily dose of 1 mg/kg wild type NT-4/5 significantly reduced (about 50% on average) of both blood glucose level (P<0.05) and triglyderide level (P<0.01). One way ANOVA followed by Dunnett's test was used for statistical analysis.

Pegylated mutant NT-4/5 (Q105C) preserved TrkB receptor agonist activity in the cell based assay, however it was inactive in vivo (no effect on body weight, food intake, glucose or triglyceride of NONcNZO-10 polygenic obese mice at 2 mg/kg/week injection). Pegylated NT-4/5(E67C) was also active in activating TrkB receptor in the cell based assay.

### Example 7

### Effect of Pegylated NT-4/5 on Carbohydrate, Lipid and Body Weight Homeostasis in the db/db Monogenic Obese Mice

### A. Experimental Protocol

Test animals: 30 female db/db mice at 7 weeks of age weighing in the range of 27-34 g were used in this study. These mice were housed in a temperature (19.5-24.5° C.) and relative humidity (45-65%) controlled room with a 12-hour light/dark cycle, with ad libitum access to filtered tap-water and irradiated pelleted laboratory chow (PURINA) throughout the study. Upon receipt at animal facilities, they were housed 1 per cage covered with filtered caps and at least a 5-day acclimation period was observed.

Methods of making pegylated NT-4/5: A mutation was introduced to the glycine 1 position of mature human NT-4/5 sequence to serine, resulting in a mutant form of NT-4/5 (NT4-G1S) in order to generate a PEG attaching site. NT4-G1S protein was expressed in E. coli, solubilized, extracted, purified and refolded as described above. Refolded NT4-G1S was dialyzed into 50 mM sodium phosphate buffer at pH 6.8 and treated with 1.5 molar excess sodium meta-periodate for 10 minutes to oxidize the N-terminal serine (or threonine) residue to an aldehyde. Remaining periodate was removed by dialysis against 30 mM sodium acetate at pH 4.5. The exact concentration of NT4-G1S was determined and a 2 to 4 fold molar excess of 20K-m-PEG-HZ (methoxy polyethylene glycol hydrazide) (MW of 20 kDa, SunBio, South Korea) was added. The reaction was incubated at 37° C. for several days with gentle agitation. Unconjugated PEG, pegylated NT4-G1S and non-pegylated NT4-G1S were separated via ion exchange or size exclusion chromatography.

Assays of TrkB specific activation by pegylated NT-4/5: Stable cell lines expressing human TrkA, TrkB or TrkC were used to determine the functional activity, i.e. receptor phosphorylation, of NT-4/5 and different forms of pegylated NT-4/5 as previously described (Sadick et al. Experimental Cell Research 234: 354-361, 1997).

Administration of NT-4/5 and pegylated NT-4/5: Wild type NT-4/5 was obtained from Genentech Inc. The mice were divided into 5 groups of equivalent body weight distribution (6 mice per group). Each group received on the first day (day 1) of the study one and the only dose of either vehicle, 5 mg/kg of NT-4/5 produced as described above, 5 mg/kg of 2PEG-NT4-G1S, or 5 mg/kg of 1PEG-NT4-G1S. 2PEG-NT4-G1S represents one PEG molecule was conjugated to each NT-4/5 monomer in a NT-4/5 dimer, and 1PEG-NT4-G1S represents one PEG molecule was conjugated to only one monomer of the NT-4/5 dimer. 2PEG-NT4-G1S and 1PEG-NT4-G1S were separated by ion exchange. Four days before starting the treatment (day −4), blood samples were collected from the mice as described previously for the determination of baseline levels of serum glucose. On day 4 after the treatment started, blood samples were collected again to determine serum glucose level. Body weight and food intake were monitored 5 days a week through out the entire study.

### B. Results

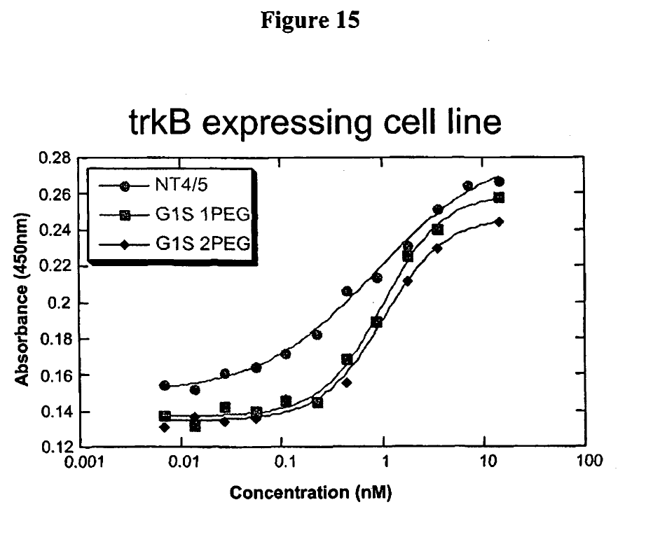

As shown in FIG. 15, native NT-4/5, 2PEG-NT4-G1S and 1PEG-NT4-G1S increased receptor tyrosine phosphorylation of TrkB-expressing cell line in a dose dependent manner. But native NT-4/5, 2PEG-NT4-G1S and 1PEG-NT4-G1S did not increase receptor tyrosine phosphorylation of trkA- or trkC-expressing cell line. As a control for the experiment, NGF activated trkA expressing cells and NT-3 activated trkC expressing cells.

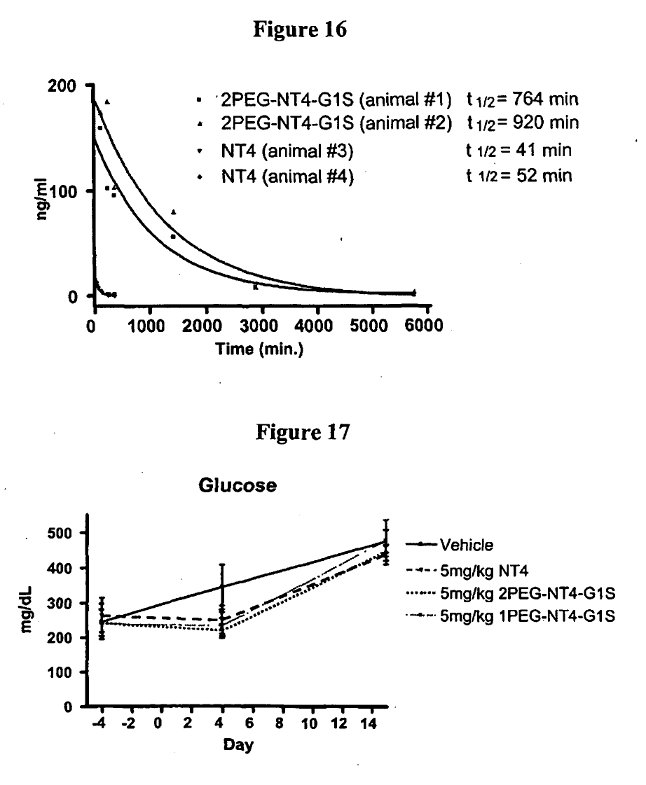

In addition, pegylated NT-4/5 has extended half life in vivo. As shown in FIG. 16, the serum half life of 2PEG-NT4-G1S was estimated to be 764-960 minutes after a single subcutaneous dose of 4 mg/kg in the db/db mice. The half life of native NT-4/5 was approximately 41-52 minutes. The absolute protein levels of 2PEG-NT4-G1S were also several orders of magnitude higher than those of native NT-4/5 at any given time point.

As shown in Tables 21 and 22 below, a single subcutaneous injection of NT-4/5 and pegylated NT-4/5 (2PEG-NT4-G1S and 1PEG-NT4-G1S) reduced the body weight growth and food intake of db/db mice significantly. There was no significant difference between NT-4/5 and pegylated NT-4/5 in this experiment using femal db/db mice given a single 5 mg/kg dose of the proteins.

TABLE 21
Body weight expressed in percentage of baseline value after a
single injection of Vehicle, NT-4/5 or pegylated NT-4/5.

| | Day 1 | Day 2 | Day 3 | Day 4 |
|---|---|---|---|---|
| Vehicle | 100.0 | 101.7 | 104.6 | 106.2 |
| | 0.0 | 0.8 | 0.9 | 1.1 |
| NT-4/5 | 100.0 | 97.7 | 100.5 | 101.7 |
| | 0.0 | 0.7*** | 0.5*** | 0.8*** |
| 2PEG-NT4-G1S | 100.0 | 97.3 | 98.4 | 100.1 |
| | 0.0 | 1.0*** | 0.7*** | 0.9*** |
| 1PEG-NT4-G1S | 100.0 | 98.2 | 100.0 | 103.0 |
| | 0.0 | 0.8** | 0.0*** | 0.6** |

Value are expressed as mean +/− SEM
Statistics: Two way analysis of variance (ANOVA) followed by a Bonferroni's post-test as compared to vehicle group
*P < 0.05
**P < 0.01
***P < 0.001

TABLE 22
Daily food intake (g/day) after a single injection of Vehicle,
NT-4/5 or pegylated NT-4/5.

| | Day 1 | Day 2 | Day 3 | Day 4 |
|---|---|---|---|---|
| Vehicle | 6.9 | 6.7 | 8.3 | 8.0 |
| | 0.2 | 0.2 | 0.3 | 0.6 |
| NT-4/5 | 6.6 | 5.2 | 7.3 | 6.3 |
| | 0.3 | 0.3* | 0.6 | 0.3* |
| 2PEG-NT4-G1S | 6.7 | 5.3 | 5.8 | 6.2 |
| | 0.3 | 0.5 | 0.8*** | 0.2** |
| 1PEG-NT4-G1S | 6.8 | 5.3 | 6.7 | 6.3 |
| | 0.1 | 0.2 | 0.3* | 0.3* |

Value are expressed as mean +/− SEM
Statistics: Two way analysis of variance (ANOVA) followed by a Bonferroni's posttest as compared to vehicle group
*P < 0.05
**P < 0.01
***P < 0.001

As shown in FIG. 17, a single subcutaneous injection of NT-4/5 and pegylated NT-4/5 (2PEG-NT4-G1S and 1PEG-NT4-G1S) showed a tendency to reduce non-fasting serum glucose levels of db/db mice after 4 days. However the relative lack of efficacy in glucose reduction may be related to the fact that these db/db mice were much younger than those used in prior studies and that they were not yet diabetic at the beginning of this study.

### Example 8

### Differential Effects of Pegylated NT-4/5 Versus Wild Type (WT) NT-4/5 on Body Weight Homeostasis and Food Intake in the DIO Mice

### A. Experimental Protocol

Test animals: Mice with diet induced obesity (DIO) were used in this study. 29 male C57BL/6J mice were weaned at 4 weeks of age. Immediately after weaning, they were put on 58% high fat diet (D12331i, Research Diet) through out the study. The DIO mice at 13 weeks of age weighing in the range of 28-40 g were used in this study. These mice were housed in a temperature (19.5-24.5° C.) and relative humidity (45-65%) controlled room with a 12-hour light/dark cycle, with ad libitum access to filtered tap-water and 58% high fat diet (D12331i, Research Diet) throughout the study. Upon receipt at animal facilities, they were housed 1 per cage covered with filtered caps and at least a 5-day acclimation period was observed.

Administration of NT-4/5 and pegylated NT-4/5: NT-4/5 was obtained from Genentech Inc. or produced as described in U.S. Pat. No. 6,184,360. Pegylated NT-4/5 was produced as follows: A mutation was introduced by changing the glycine 1 position of mature human NT-4/5 sequence to serine, resulting in a mutant form of NT-4/5 (NT4-G1S). NT4-G1S protein was expressed in E. coli, solubilized, extracted, purified and refolded as described above. Refolded NT4-G1S was dialyzed into 50 mM sodium phosphate buffer at pH 6.8 and treated with 1.5 molar excess sodium meta-periodate for 10 minutes to oxidize the N-terminal serine (or threonine) residue to an aldehyde. Remaining periodate was removed by dialysis against 30 mM sodium acetate at pH 4.5. The exact concentration of NT4-G1S was determined and a 2 to 4 fold molar excess of 20K-m-PEG-HZ (methoxy polyethylene glycol hydrazide) was added. The reaction was incubated at 37° C. for several days with gentle agitation. Unconjugated PEG, pegylated NT4-G1S and non-pegylated NT4-G1S were separated via ion exchange or size exclusion chromatography.

The mice were divided into 3 groups of equivalent body weight and baseline blood glucose distribution (7-8 mice per group). Each group received four weekly intravenous doses (on day 1, 8, 15 and 22) of either vehicle, 2 mg/kg of NT-4/5 or 2 mg/kg of pegylated G1S NT-4/5 respectively. Body weight and food intake were monitored 5 days per week through out the entire study.

### B. Results:

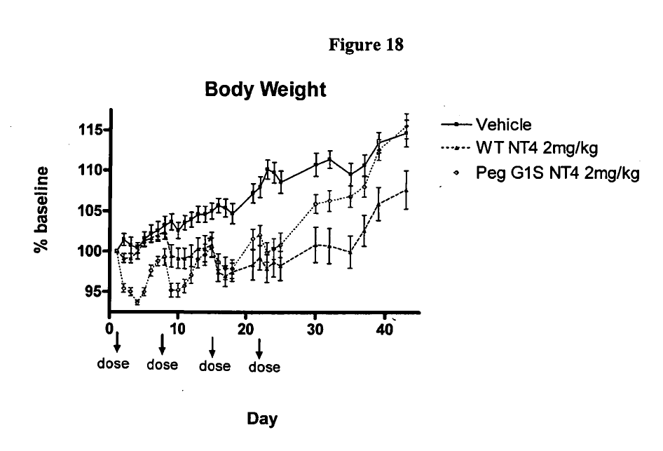

As shown in FIG. 18, the body weights of the treatment groups (both wildtype NT-4/5 and pegylated G1S NT-4/5) differed significantly by 2 way ANOVA (F=207.01, P<0.0001). Bonferroni posttests showed significant pairwise differences between WT NT-4/5 group with the vehicle control group (P<0.001 from day 16 to day 38; P<0.01 on day 43; not significant at all the other time points) as well as between pegylated G1S NT-4/5 and vehicle control (P<0.05 on days 2-3; P<0.01 on days 4-5; P<0.01 from day 9 to 12; P<0.01 on day 16-18; P<0.05 on day 21 and 22; P<0.001 on day 23-25; not significant at all the other time points). The first single dose of pegylated G1S NT-4/5 was more effective in reducing body weight. Body weight recovery occurred rapidly within 3-4 days after each weekly dosing of pegylated GIS NT-4/5. By contrast, long term weekly dosing of wildtype (WT) NT-4/5 was more effective in maintaining lower body weight.

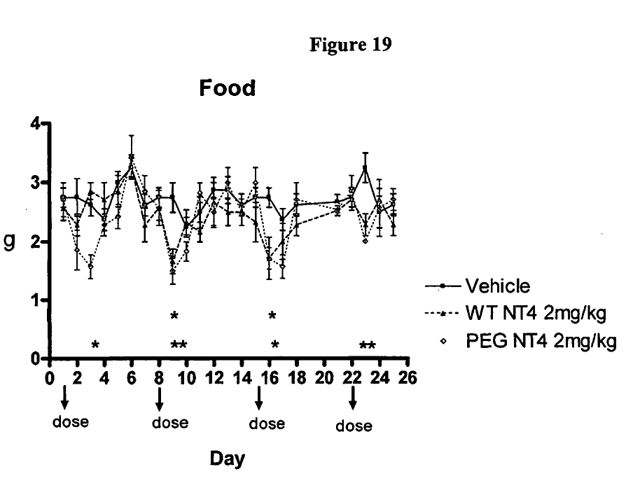

As shown in FIG. 19, the food intake of the treatment groups differed significantly by 2 way ANOVA (F=11.62, P<0.0001). Bonferroni posttests showed significant pairwise difference between each treatment group with the vehicle control group (* P<0.05 and ** P<0.01 as indicated in the graph). The first single dose of pegylated G1S NT-4/5 was more effective in reducing food intake than WT NT-4/5.

### Example 9

### Effect of Daily Subcutaneous Dosing of Wt NT-4/5 on Respiratory Quotients and Body Composition in DIO Mice

### A. Experimental Protocol

Test animals: Mice with diet induced obesity (DIO) were used in this study. 29 male C57BL/6J mice were weaned at 4 weeks of age. Immediately after weaning they were put on 58% high fat diet (D12331i, Research Diet) through out the study. The DIO mice at 13 weeks of age weighing in the range of 28-40 g were used in this study. These mice were housed in a temperature (19.5-24.5° C.) and relative humidity (45-65%) controlled room with a 12-hour light/dark cycle, with ad libitu access to filtered tap-water and 58% high fat diet (D12331i, Research Diet) throughout the study. Upon receipt at animal facilities, they were housed 1 per cage covered with filtered caps and at least a 5-day acclimation period was observed.

Administration of NT-4/5: NT-4/5 was obtained from Genentech Inc. or produced as described in U.S. Pat. No. 6,184,360. The mice were divided into 2 groups of equivalent body weight distribution (8 mice per group). The mice were acclimated individually in the Comprehensive Cage Monitoring System (CCMS, Columbus Instruments) for 2 days, in preparation for the indirect calorimetry, before the treatment started. Each group received 5 daily subcutaneous doses of either vehicle or 10 mg/kg of NT-4/5, respectively. Body weight and food intake were monitored 5 days per week through out the entire study. The CCMS monitoring and data analysis of oxygen consumption and carbon dioxide production were carried out according published and commonly accepted protocols as described at website of Jackson Laboratory. The body composition, i.e. percent lean body mass, fat content, and bone density was determined the day both before treatment started (day 0) and the final day of treatment (day 5) with a PIXImus Mouse Densitometer (GE Lunar Medical Systems, Madison, Wis.) using software version 1.46. Brommage et al., Am. J. Physiol. Endocrinol. Metab. 285: 454-459 (2003).

### B. Results:

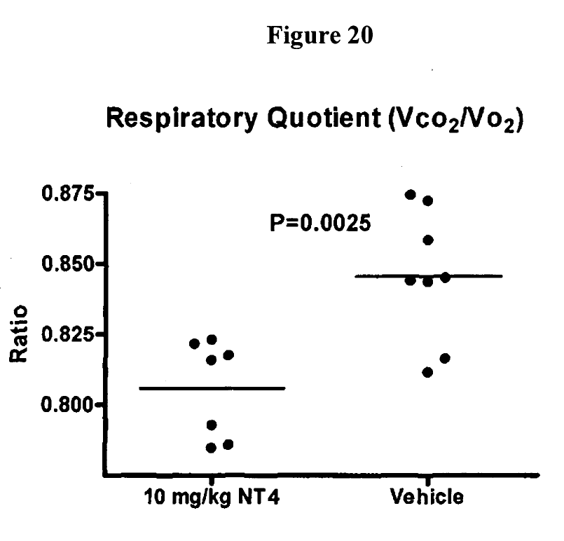

As shown in FIG. 20, daily subcutaneous injection of NT-4/5 (10 mg/kg) significantly (P=0.0025, Student t-test) reduced the respiratory quotient (RQ=Vco₂/Vo₂, i.e. ratio of carbon dioxide production over oxygen consumption measured by CCMS). Since the body mainly uses fat (RQ˜0.67) and carbohydrate (RQ=1) for oxidation at the resting state, this reduction in RQ indicates a shift to increased utilization of body fat (fatty acids, triglycerides and cholesterol etc.) relative to carbohydrate as the major fuel source.

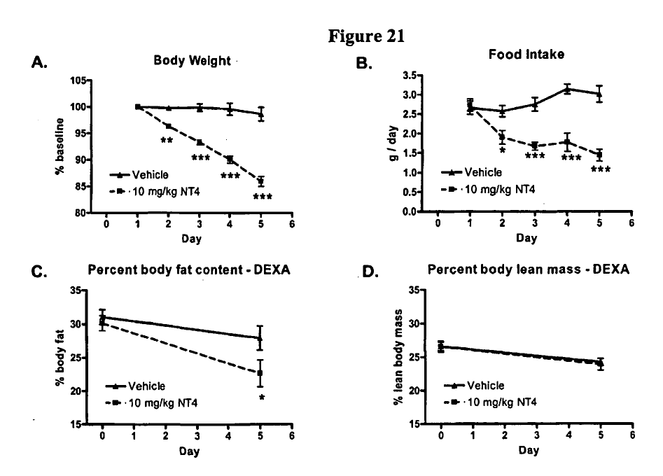

As shown in FIG. 21, daily subcutaneous injection of NT-4/5 (10 mg/kg) significantly reduced the body weight (panel A), food intake (panel B) and body fat content (panel C), but not the lean body mass (panel D).

Bone density in the NT-4/5 (10 mg/kg) treated group was compared to the vehicle group. Bone mineral density of the NT-4/5 treated group was 0.5235±0.0158 g/cm² (mean±SEM) on day 0, and 0.5452±0.0142 g/cm² (mean±SEM) on day 5. Bone mineral density of the vehicle group was 0.5070±0.0210 g/cm² (mean±SEM) on day 0, and 0.4698±0.0198 g/cm² (mean±SEM) on day 5. The difference of bone density of the NT-4/5 treated group was significantly different from that of the vehicle on day 5 (P<0.05, 2-way ANOVA with Bonferroni's test). The reduction of bone density of the vehicle group was probably due to the stress of being housed in the small confined space in the CCMS over 5 days, as confirmed by extremely high levels of serum corticosterone found in both the vehicle and NT-4/5 treated groups on day 5. This study suggests that NT-4/5 treatment prevents stress-induced bone loss in the presence of diet-induced obesity.

No change of estimated cumulative energy expenditure from day 1 to day 5 was observed in NT-4/5 (10 mg/kg) treated group as compared to the vehicle control group based on the rate of oxygen consumption measured by indirect calorimetry (http:/pga.jax.org/protocol_{—}012.html)

### Example 10

### Differential Potency of NT-4/5 Versus BDNF on the Body Weight and Food Intake of DIO Mice

### A. Experimental Protocol

Test animals: Mice with diet induced obesity (DIO) were used in this study. 22 male C57BL/6J mice were weaned at 4 weeks of age. Immediately after weaning they were put on 58% high fat diet (D12331i, Research Diet) through out the study. The DIO mice at 13 weeks of age weighing in the range of 28-40 g were used in this study. These mice were housed in a temperature (19.5-24.5° C.) and relative humidity (45-65%) controlled room with a 12-hour light/dark cycle, with ad libitum access to filtered tap-water and 58% high fat diet (D12331i, Research Diet) throughout the study. Upon receipt at animal facilities, they were housed 1 per cage covered with filtered caps and at least a 5-day acclimation period was observed.

Administration of NT-4/5 and BDNF: NT-4/5 was produced was produced as described in U.S. Pat. No. 6,184,360. The recombinant human BDNF was obtained from PeproTech Inc. (Rocky Hill, N.J.). The BDNF material was tested by the vendor to be active in two biological assays: (a) induction of choline acetyltransferase activity in primary rat basal forebrain septal culture (ED50=25-50 ng/mL) and (b) induction of neurite outgrowth of chick embryonic day 8 trigeminal neurons (ED50=0.1-1 ng/mL).

The mice were divided into 3 groups of equivalent body weight distribution (7-8 mice per group). Each group received 2 weekly intravenous doses of either vehicle, 2 mg/kg of NT-4/5 or 2 mg/kg of BDNF, respectively. Body weight and food intake were taken the day before treatment started (day 0) and followed subsequently on a daily basis.

### B. Results:

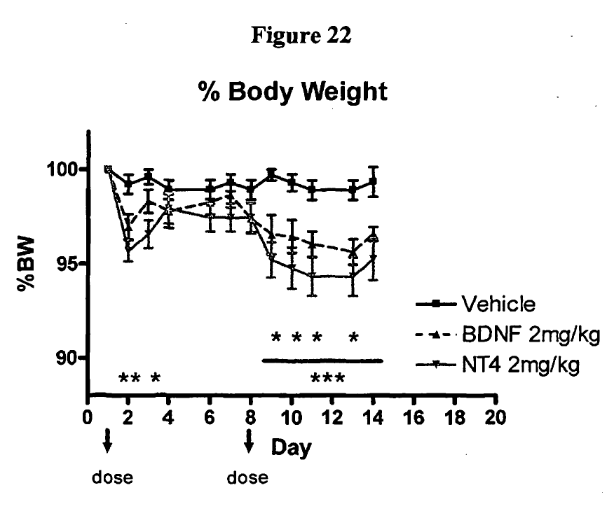

As shown in FIG. 22, weekly intravenous injection of NT-4/5 (2 mg/kg) reduced the body weight more effectively than that of BDNF (2 mg/kg). For example, on day 2, NT-4/5 at 2 mg/kg reduced body weight significantly, but BDNF at 2 mg/kg did not significantly reduce body weigh.

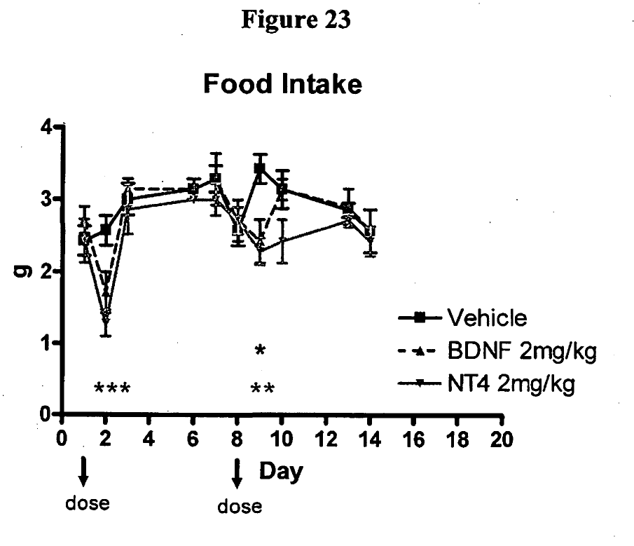

As shown in FIG. 23, weekly intravenous injection of NT-4/5 (2 mg/kg) reduced the food intake more effectively than that of BDNF (2 mg/kg). For example, on day 2, NT-4/5 at 2 mg/kg reduced food intake significantly, but BDNF at 2 mg/kg did not significantly reduce food intake.

### Example 11

### Activity of WT NT-4/5 on the Body Weight and Food Intake in Normal Lean Mice

### A. Experimental Protocol

Test animals: Mice with normal diet were used in this study. Forty male C57BL/6J mice weighed 25-31 g at 12 weeks of age were housed in a temperature (19.5-24.5° C.) and relative humidity (45-65%) controlled room with a 12-hour light/dark cycle, with ad libitum access to filtered tap-water and regular mouse chow (6% fat diet) throughout the study. Upon receipt at animal facilities, they were housed 1 per cage covered with filtered caps and at least a 5-day acclimation period was observed.

Administration of NT-4/5: NT-4/5 was produced as described in U.S. Pat. No. 6,184,360. The mice were divided into 5 groups of equivalent baseline body weight, food intake and nonfasting blood glucose level distribution (8 mice per group). Each group received 4 daily subcutaneous doses of vehicle, 1 mg/kg, 2 mg/kg, 5 mg/kg or 10 mg/kg of NT-4/5, respectively, from day 1 to day 4. Body weight and food intake were monitored on daily basis. The non-fasting blood glucose was measure as previously described once a week (day 1 and day 8).

### B. Results:

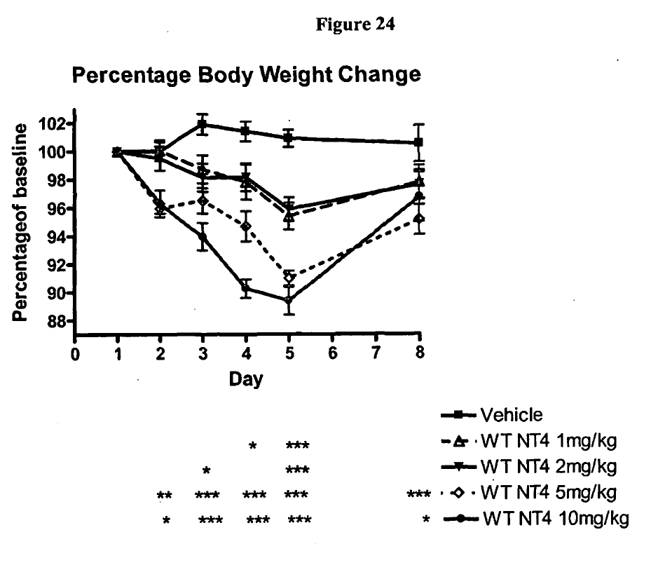

As shown in FIG. 24, daily subcutaneous injection of NT-4/5 (1, 2, 5 and 10 mg/kg) significantly reduced the body weight in a dose dependent manner as compared to the vehicle control group.

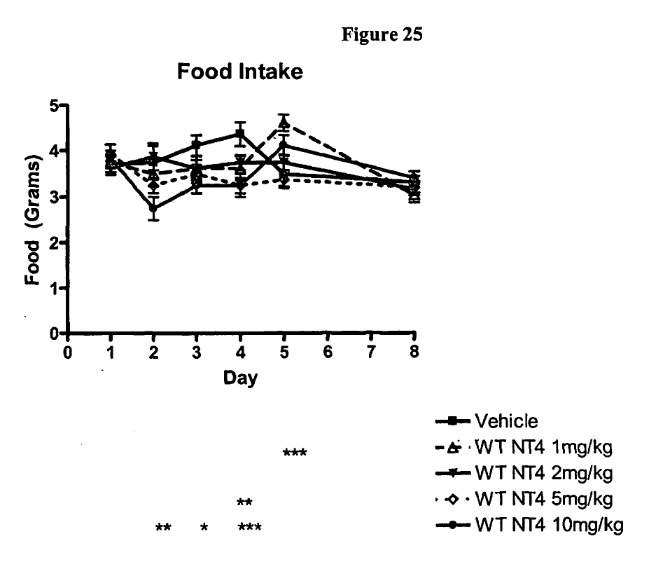

As shown in FIG. 25, daily subcutaneous injection of NT-4/5 (1, 5 and 10 mg/kg) significantly reduced food intake in a dose dependent manner as compared to the vehicle control group.

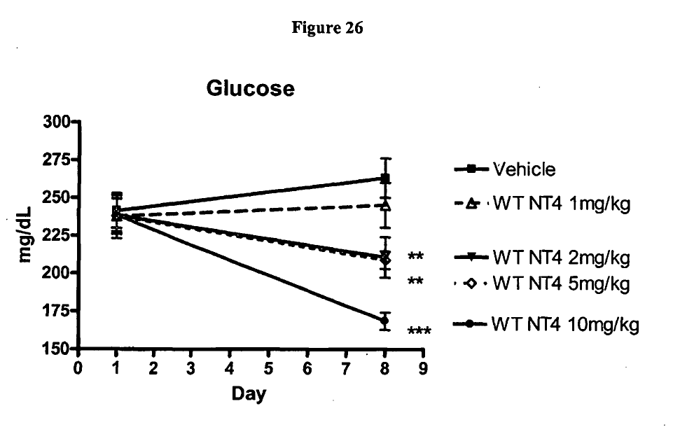

As shown in FIG. 26, daily subcutaneous injection of NT-4/5 (2, 5 and 10 mg/kg) significantly reduced nonfasting blood glucose in a dose dependent manner as compared to the vehicle control group.

Blood triglyceride levels were also measured. Daily subcutaneous injection of NT-4/5 (1, 2, 5 and 10 mg/kg) significantly reduced blood triglyceride in a dose dependent manner as compared to the vehicle control group.

Similar experiments were also performed in female C57BL/6 mice. Statistically significant weight loss in a dose dependent manner (about 8% for 5 mg/kg, and about 11% for 10 mg/kg on day 2) as compared to the vehicle control group was also observed.

### Example 12

### Effects of NT-4/5 in Survival Time in db/db Mice

### A. Experimental Protocol

Db/db female mice maintained as described in Example 1 were divided into two groups. Group 1 (8 mice) were administered with vehicle (PBS). Group 2 (6 mice) was each administered with NT-4/5 at the doses of 20 mg/kg. Both the vehicle and NT-4/5 were administered subcutaneously once daily from day 1 to day 26, and were then followed up for survival. Mortality is defined by the body wasting below 60% of the starting body weight (i.e., from about 40 g down to about 24 g) or natural death, whichever happened first.

### B. Results

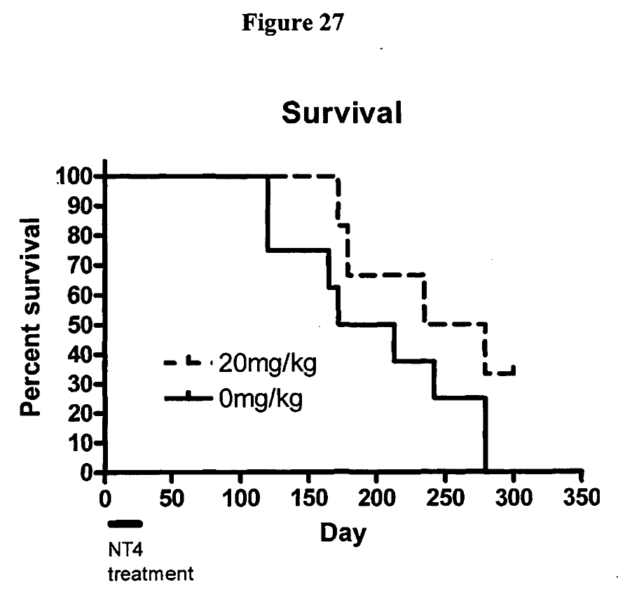

As shown in FIG. 27, the median length of survival is 257 days (n=6) for NT-4/5 (20 mg/kg) group, while it is 192.5 days (n=8) for the vehicle control group. This 65 days difference is not statistically significant (Logrank test, Chi square=2.26, P=0.1331) probably because the number of animals tested is not large enough.

### Example 13

### Effect of NT-4/5 Administration on Pain

To test whether NT-4/5 elicits pain in animals after NT-4/5 administration, a heat/thermal hyperalgesia animal model was used for testing. Adult male Sprague-Dawley rats (from Charles River) were subcutaneously injected with vehicle (PBS), NGF (2 mg/kg), or NT-4/5 (2 mg/kg). Then the latency for foot withdrawal in response to a constant radiant heat source was measured. See, Hargreaves et al., Neurology 48:501-505 (1997); Shu et al., Pain 80:463-470 (1999). No statistically significant changes of latency for foot withdrawal in response to a constant radiant heat source were observed in vehicle group and NT-4/5 treated group at 3, 5, and 24 hour after administration. However, NGF treated group showed a reduction (about 40% to 50%) of latency reduction at 3, 5, and 24 hour after administration. These data indicate that, unlike NGF, NT-4/5 at 2 mg/kg did not cause pain in these animals.

### Example 14

### Effects of NT-4/5 on Long Term Glucose Control

### A. Experimental Protocol

Db/db mice maintained as described in Example 1 were divided into five groups (groups 1-5). Group 1 were administered with vehicle (PBS). Group 2, group 3, group 4, group 5 were each administered with NT-4/5 at the doses of 2 mg/kg, 5 mg/kg, 10 mg/kg, and 20 mg/kg, respectively. Both the vehicle and NT-4/5 were administered to each group of the mice subcutaneously once daily from day 1 to day 26. One day before beginning the treatment (day 0), blood samples were collected from the mice as described previously for the determination of baseline levels of serum biomarkers (glucose and insulin). On day 45, HbA1c level was measured. Glucose tolerance test was performed on day 54 (28 days after the last dose of NT-4/5 treatment). Intraperitoneal (IP) glucose tolerance test was conducted by fasting the mice overnight (16 hours) before giving them an IP injection of a load of 2 grams of glucose/kg of body weight. Serum glucose and insulin were also measured.

### Results

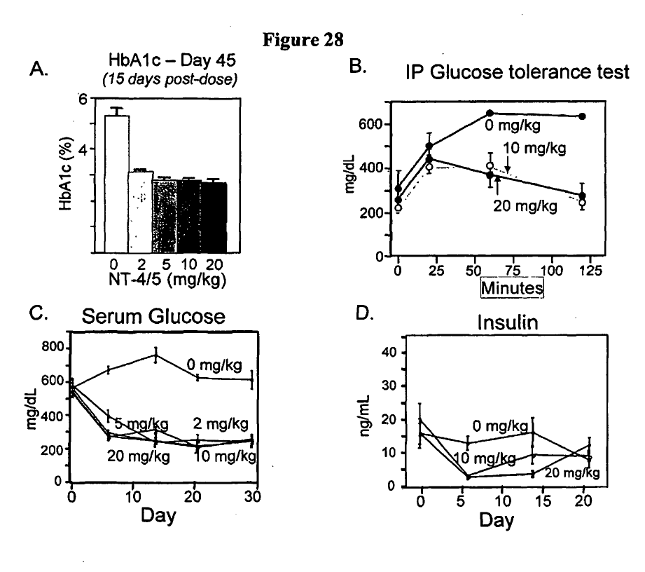

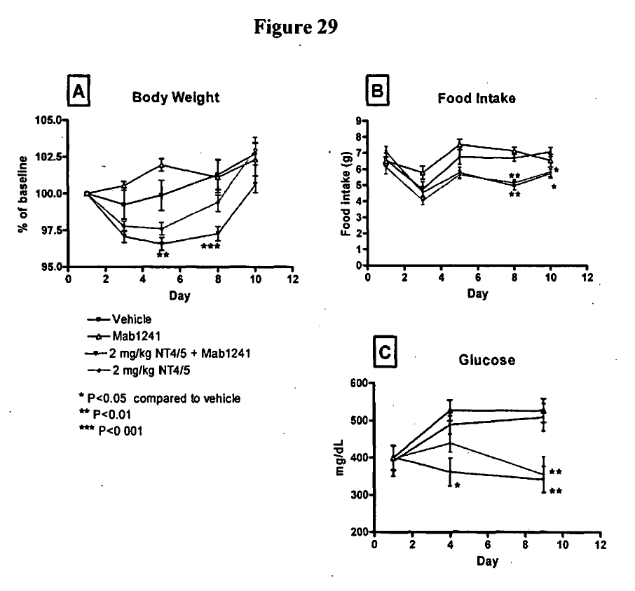

As shown in Panel A of FIG. 28, HbA1c levels in NT-4/5 treated groups (2 mg/kg, 5 mg/kg, 10 mg/kg, and 20 mg/kg) were significantly lower than the vehicle group on day 45 (15 days post-dose) after daily sc injections from day 1 to day 26. Panels B of FIG. 28 showed that NT-4/5 treatment significantly improved glucose tolerance. As shown in Panel C of FIG. 28, non-fasting serum glucose level was reduced more than 50% after NT-4/5 treatment for all treatment groups and remained low until day 29. Panel D of FIG. 28 showed significant reduction of serum insulin levels by 10 mg/kg/d of NT-4/5 treated group (on day 6, P<0.05) and 20 mg/kg/d of NT-4/5 treated group (on day 6 and day 14, P<0.05). These data indicate that NT-4/5 improved long term glucose control in db/db mice.

### Example 15

### Effects of Co-Administering Mouse Monoclonal NT4/5 Antibody Mab1241 with NT4/5 on the Biological Activities of NT4/5 in an Obese Diabetic Mouse Model

### A. EXPERIMENTAL PROTOCOL

Test animals: 32 Leptin receptor mutant db/db mice (BSK.Cg-m+/+Leprdb) obtained from Jackson Laboratory were used for the study. The mice were divided into four groups of 8 animals each (groups 1-4), with homogeneous initial body weight and glycemia values based on a randomization table. The mice were housed one per cage.

Anti-NT4/5 and NT4/5: Both anti-NT4/5 monoclonal antibody Mab 1241 and human NT4/5 were obtained from Genentech. According to BIAcore assay, the binding affinity between the monovalent Fab fragment of Mab1241 and NT4/5 was 170 nM with kon=2.2×105M-1s-1 and koff=0.039s⁻¹. The Mab1241 Fab fragment does not interfere with the ability of NT4/5 to bind to human TrkB receptor in a surface plasmon resonance BIAcore assay. (data not shown).

Administration of Mab 1241 and NT4/5: On day one, mice in groups 1-4 were treated with vehicle (PBS) subcutanouesly (s.c.), 5 mg/kg Mab 1241 intraperitioneally (i.p.), 5 mg/kg Mab 1241 intraperitioneally (i.p.) plus 2 mg/kg NT4/5 subcutanouesly (s.c.), and 2 mg/kg NT4/5 subcutanouesly (s.c.), respectively. On day 2 through day 5, group 1 was treated with a daily dose of vehicle (PBS) subcutanouesly (s.c.). Groups 3 and 4 were treated with a daily dose of 2 mg/kg NT4/5 subcutanouesly (s.c.).

Collection of samples for analysis: one day before the beginning of the treatments (day 0), non-fasted mice were weighed and blood samples were collected through the retro-orbital plexus (about 300 ul/mouse) under isoflurane anesthesia. Baseline non-fasted blood glucose level was measured by One-Touch Ultra glucose meter from the tail bleed.

### B. RESULTS

Effect on body weight: The body weights of each group of mice were taken every other day starting from day 1, and the results are graphically presented in FIG. 27A. As shown in the figure, the treatment of daily 2 mg/kg NT4/5 from day 1 to day 5 produced a small, but statistically insignificant reduction in body weight. The Mab 1241 injection alone had no effect on body weight. By contrast, co-treatment of daily 2 mg/kg NT4/5 and a single injection of Mab 1241 on day 1 induced a significant reduction in body weight on day 5 and on day 8. Two-way ANOVA with pairwise comparisons by Bonferrioni posttests of each treatment relative to the vehicle control group was used for statistical analysis.

Effect on food intake: The food intakes of each group of mice were taken every other day starting from day 1, and the results are graphically presented in FIG. 27B. As shown in the figure, the treatment of daily 2 mg/kg NT4/5 from day 1 to day 5 significantly reduced food intake on day 8 and day 10. However, Mab co-treatment did not significantly change the food intake effect induced by 2 mg/kg NT4/5 alone. Two-way ANOVA with pairwise comparions by Bonferrioni posttests of each treatment relative to the vehicle control group was used for statistical analysis.

Effect on glucose level: blood glucose levels of the mice were assayed on day 1, day 4 and day 9 using the method described above. The results are graphically represented in FIG. 27C. As shown in the figure, the treatment of daily 2 mg/kg NT4/5 from day 1 to day 5 reduced non-fasted glucose level only on day 9, but not on day 4. In contrast, co-treatment of daily 2 mg/kg NT4/5 and a single injection of Mab 1241 on day 1 reduced non-fasted glucose level as early as day 4. The glucose level of the co-treatment group remained significantly lower than the vehicle group on day 9. Two-way ANOVA with pairwise comparions by Bonferrioni posttests of each treatment relative to the vehicle control group was used for statistical analysis.

### C. CONCLUSION

This experiment demonstrates that co-administration of a low affinity NT4/5 antibody Mab 1241 significantly enhances the therapeutic efficacy of daily dosing of NT4/5 protein in terms of controlling body weight and blood glucose level in db/db mice.

It is understood that the examples and embodiments described herein are for illustrative purposes only and that various modifications or changes in light thereof will be suggested to persons skilled in the art and are to be included within the spirit and purview of this application. All publications, patents and patent applications cited herein are hereby incorporated by reference in their entirety for all purposes to the same extent as if each individual publication, patent or patent application were specifically and individually indicated to be so incorporated by reference.

1. A method for treating obesity in an individual, said method comprising administering to said individual an effective amount of a neurotrophin-4/5 (NT-4/5) polypeptide, wherein the individual does not have genetic deficiency of brain-derived neurotrophic factor (BDNF).

2. The method of claim 1, wherein the individual has leptin-resistance.

3. The method of claim 1, wherein a disorder associated with obesity is treated in the individual, and the disorder is selected from the group consisting of hyperglycemia, low glucose tolerance, insulin resistance, lipid disorder, dyslipidemia, hyperlipidemia, hypertriglyceridemia, and metabolic syndrome.

4. The method of claim 1, wherein the individual has a reduction of body fat content as a result of the treatment.

5. The method of claim 1, wherein said NT-4/5 polypeptide is a naturally occurring NT-4/5.

6. The method of claim 1, wherein said NT-4/5 polypeptide comprises the amino acid sequence of SEQ ID NO:1.

7. The method of claim 1, wherein said NT-4/5 polypeptide comprises the amino acid sequence of SEQ ID NO:1 with an amino acid substitution.

8. The method of claim 1, wherein said NT-4/5 polypeptide is linked to a polyethylene glycol molecule (PEG).

9. The method of claim 8, wherein said NT-4/5 polypeptide comprises the amino acid sequence of SEQ ID NO:1 with amino acid residue G at position 1 changed to S or T, and wherein said NT-4/5 polypeptide is linked to a PEG molecule at position 1.

10. (canceled)

11. A method for reducing body weight or preventing weight gain in an individual, said method comprising administering to said individual an effective amount of an NT-4/5 polypeptide, wherein the individual does not have genetic deficiency of BDNF.

12. The method of claim 11, wherein said NT-4/5 polypeptide is a naturally occurring NT-4/5.

13. The method of claim 11, wherein said NT-4/5 polypeptide comprises the amino acid sequence of SEQ ID NO:1.

14. The method of claim 11, wherein said NT-4/5 polypeptide comprises the amino acid sequence of SEQ ID NO:1 with an amino acid substitution.

15. The method of claim 11, wherein said NT-4/5 polypeptide is linked to a polyethylene glycol molecule (PEG).

16. The method of claim 15, wherein said NT-4/5 polypeptide comprises the amino acid sequence of SEQ ID NO:1 with amino acid residue G at position 1 changed to S or T, and wherein said NT-4/5 polypeptide is linked to a PEG molecule at position 1.

17-36. (canceled)

37. The method of claim 1, wherein the individual is at risk of obesity.

38. The method of claim 1, wherein the individual is overweight.

39. The method of claim 11, wherein the individual is at risk of obesity.

40. The method of claim 11, wherein the individual is overweight.


SEQUENCE LISTING (2 sequences)

SEQ ID NO: 1 - protein, 130 aa, Homo sapiens
GVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 2 - DNA, 394 bp, Homo sapiens
ggggtgagcgaaactgcaccagcgagtcgtcggggtgagctggctgtgtgcgatgcagtcagtggctgggtgacagaccgccggaccgctgtggacttgcgtgggcgcgaggtggaggtgttgggcgaggtgcctgcagctggcggcagtcccctccgccagtacttctttgaaacccgctgcaaggctgataacgctgaggaaggtggcccgggggcaggtggagggggctgccggggagtggacaggaggcactgggtatctgagtgcaaggccaagcagtcctatgtgcgggcattgaccgctgatgcccagggccgtgtgggctggcgatggattcgaattgacactgcctgcgtctgcacactcctcagccggactggccgggcctgag